(19) United States
(12) Patent Application Publication
(10) Pub. No.: US 2020/0080157 A1
(43) Pub. Date: Mar. 12, 2020

(54) PROGNOSIS AND TREATMENT OF RELAPSING LEUKEMIA

(71) Applicant: McMaster University, Hamilton (CA)

(72) Inventors: Mickie Bhatia, Ancaster (CA); Allison Boyd, Ancaster (CA); Leili Aslostovar, Hamilton (CA)

(21) Appl. No.: 16/562,792

(22) Filed: Sep. 6, 2019

Related U.S. Application Data
(60) Provisional application No. 62/728,535, filed on Sep. 7, 2018.

(51) Int. Cl. C12Q 1/6886; A61K 31/5415; G16B 40/10

(52) U.S. Cl. CPC C12Q 1/6886; A61K 31/5415; C12Q 2600/158; C12Q 2600/106; G16B 40/10

(57) ABSTRACT

Described are biomarkers and associated methods for determining a prognosis for a subject with leukemia and/or for detecting Leukemic Regenerating Cells (LRCs) or Hematopoietic Regenerating Cells. While chemotherapy may successfully deplete leukemic stem cells (LSCs), a molecularly distinct population of LRCs are observed following cytotoxic chemotherapy not seen in therapy naïve subjects with leukemia or healthy hematopoietic cell populations. Also described are methods for the treatment of leukemia that target LRCs in a subject in need thereof.

### RELATED APPLICATIONS

This application claims the benefit of priority of U.S. provisional patent application No. 62/728,535 filed on Sep. 7, 2018, the entire contents of which are hereby incorporated by reference.

### FIELD OF THE INVENTION

The present invention relates to leukemia and more specifically to methods for the prognosis and/or treatment of relapsing leukemia.

### BACKGROUND OF THE INVENTION

Cytarabine (AraC)-based chemotherapy regimens have remained the standard of care for adult acute myeloid leukemia (AML) for decades. Despite successful remission induction using this approach, the vast majority of AML patients suffer from aggressive disease recurrence within 3 years (Estey and Dohner, 2006). Through rigorous development of human-mouse xenograft assays, rare functional subsets of AML have been characterized by testing the potential to initiate patient leukemic disease in recipient mice (Leukemia Initiating Cells; LICs), and cells detected in LIC assays have been operationally defined as leukemia stem cells (LSCs) (Thomas and Majeti, 2017). It has been proposed that LSCs preferentially resist chemotherapy, providing a cellular reservoir that is thought to form the basis for relapse (Thomas and Majeti, 2017). However, this prediction is primarily based on the dormant cell cycle status of LSCs (Jordan et al., 2006), and these ideas have not been rigorously tested by analyzing leukemic populations that selectively persist post-therapy.

Several studies have carefully evaluated the functional and molecular biology of overtly relapsed AML (Ding et al., 2012; Hackl et al., 2015; Ho et al., 2016; Shlush et al., 2017), however little attention has been dedicated to exploring the initial stages of disease response to chemotherapy itself. Defining and characterizing these cells is especially difficult from patients, as the number of residual leukemic cells is drastically reduced post-treatment due to the cytoreductive nature of the therapy itself. This is further confounded by the difficulty of resolving rare primitive AML cells from endogenous normal hematopoietic stem/progenitors within patient bone marrow (BM), due to overlapping molecular and phenotypic properties (Eppert et al., 2011; Levine et al., 2015). Further challenges arise from the heterogeneous cell surface phenotypes manifested by LSCs derived from different AML patients. To resolve this issue, transcriptional signatures have recently been described that correlate to LIC capacity and are suggested to provide prognostic value for AML patient survival (Eppert et al., 2011; Ng et al., 2016). Despite these advances, the current understanding of LSCs has been rendered in the absence of chemotherapy treatment, and thus represents properties of “therapy-naive” LSCs. This leaves the acute response of AML LSCs to chemotherapy in vivo largely unknown.

Recent efforts to recreate clinically relevant chemotherapy regimes in xenografts have introduced the possibility that LSCs are in fact susceptible to standard AraC chemotherapy (Farge et al., 2017; Griessinger et al., 2014). These results unexpectedly challenge one of the founding elements of the Cancer Stem Cell (CSC) theory that CSCs are spared from cytotoxic chemotherapy (Jordan et al., 2006). Further investigation is required to reveal the sequence of events that shape leukemic regeneration activity post-therapy, leading to disease relapse.

### SUMMARY OF THE INVENTION

Despite successful remission induction, recurrence of leukemia remains a clinical obstacle thought to be caused by the retention of dormant leukemic stem cells (LSCs). Using chemotherapy-treated AML xenografts and patient samples, patient remission and relapse kinetics were modelled to reveal that LSCs are effectively depleted via cell cycle recruitment, leaving the origins of relapse unclear. Remarkably, post-chemotherapy, in vivo characterization at the onset of disease relapse has revealed a unique molecular state of Leukemic Regenerating Cells (LRCs) responsible for disease re-growth. LRCs are transient and are molecularly distinct from therapy-naive LSCs. LRCs and their molecular features can therefore be used as markers of relapse and are therapeutically targetable to prevent disease recurrence. A population of Hematopoietic Regenerating Cells (HRCs) has also been identified in subjects without leukemic disease following induced injury such as by administration of chemotherapy with cytarabine or 5-fluorouracil or following radiation.

As shown in the Examples, complementary clinical and in vivo experimental evidence challenges the widely held view that LSCs preferentially survive chemotherapy. Chemotherapy successfully depletes LSCs, creating a transient period of leukemic vulnerability, where regenerative potential must re-establish prior to overt disease recurrence. A molecular signature of active leukemic regenerating cells (LRCs), which give rise to relapsed disease but do not resemble therapy-naive LSC states, is identified including the biomarkers listed in Table 4A. Remarkably, the molecular profile of LRCs is conserved across genetically diverse cases of human AML and identifies reservoirs of minimal residual disease in clinically treated patients who ultimately progress to relapse. These findings have direct therapeutic value given the uniquely targetable nature of LRC signatures to curtail leukemic re-growth and provide markers of leukemic disease recurrence. Furthermore, screening for agents that selectively target LRCs is expected to be useful for identifying candidates for preventing or inhibiting relapsing leukemic disease.

Accordingly, in one embodiment there is provided a method of determining a prognosis for a subject who has completed a cytotoxic treatment for leukemia. In one embodiment, the method comprises:

determining a level of one or more biomarkers listed in Table 4A in a test sample obtained from the subject after completing the cytotoxic treatment for leukemia; and

comparing the level of the one or more biomarkers in the test sample to one or more control levels,

wherein a difference or similarity in the level of the one or more biomarkers in the test sample compared to the one or more control levels is indicative of whether the subject has an increased or decreased risk of relapsing leukemia.

Also provided is a method of detecting Leukemic Regenerating Cells (LRCs) in a test sample. In one embodiment, the method comprises detecting a level of one or more biomarkers listed in Table 4A in the test sample and comparing the level of the one or more biomarkers in the test sample to one or more control levels.

In one embodiment, the one or more biomarkers are selected from FASLG, DRD2, SLC2A2, and FUT3. In one embodiment, the one or more biomarkers comprises SLC2A2 and/or DRD2.

The test sample may be a biological sample comprising mononuclear cells and/or suspected of comprising leukemic cells, optionally CD45+ cells. In one embodiment, the test sample comprises CD34+ cells, or CD34+CD38− cells. In one embodiment, the test sample comprises blood, fractionated blood, or bone marrow.

The prognostic method described herein are useful for providing information on the likelihood of relapse in a subject having completed a cytotoxic treatment for leukemia. As shown in the Examples, completing a cytotoxic treatment such as chemotherapy results in a transient period of leukemic vulnerability in which LRCs emerge and may give rise to relapsed disease, while LRCs are not observed in therapy-naïve subjects. LRCs were observed following induced injury with 5-fluorouracil or cytarabine, as well as with radiation.

A population of Hematopoietic Regenerating Cells (HRCs) was observed in subjects without cancer that received a cytotoxic treatment such as chemotherapy or radiation. In one embodiment, the cytotoxic treatment is an induced injury comprising the administration of a chemotherapeutic agent and/or radiation. Optionally, the cytotoxic treatment comprises the use or administration of cytarabine, anthracycline or 5-fluorouracil.

Optionally In one embodiment, the method comprises generating a biomarker expression profile for the test sample based on the levels of a plurality of the biomarkers in the test sample. In one embodiment, the method comprises comparing the biomarker expression profile for the test sample to a control biomarker expression profile to determine a prognosis for the subject.

In one aspect, there is provided a computer-implemented method for determining a prognosis of a subject a subject who has completed a cytotoxic treatment for leukemia. In one embodiment, the method comprises obtaining a biomarker expression profile for a test sample from the subject based on a level of one or more biomarkers listed in Table 4A. In one embodiment, the sample was obtained from the subject after completing the cytotoxic treatment for leukemia. Optionally, the methods described herein may include obtaining a sample from the subject after completing the cytotoxic treatment. In one embodiment, the method comprises classifying, on a computer, whether the subject has a good prognosis and a low risk of relapsing leukemia or a poor prognosis and a high risk of relapsing leukemia, based on the biomarker expression profile for the test sample. Optionally, the computer-implemented method comprises generating a risk score for the subject indicative of the subject's prognosis.

Also provided is a computer system configured for implementing a prognostic method as described herein. For example, in one embodiment the system comprises a processor configured for generating or receiving a biomarker expression profile for a test subject and comparing the biomarker expression profile to a control. In one embodiment, the processor is configured for classifying whether the subject has a good prognosis and a low risk of relapsing leukemia or a poor prognosis and a high risk of relapsing leukemia based on a difference or similarity between the biomarker expression profile of the test sample and the control.

Also provided is a method of treating a subject having leukemia. In one embodiment, the method comprises determining a prognosis of the subject according to a method described herein and providing a suitable cancer treatment to the subject in need thereof according to the prognosis determined. In one embodiment, the leukemia is acute myeloid leukemia (AML).

In one embodiment, there is provided a method of treating leukemia in a subject in need thereof, the method comprising administering to the subject an agent that targets Leukemic Regenerating Cells (LRCs), wherein the subject has completed a cytotoxic treatment for leukemia. Also provided is the use of an agent that targets LRCs for preventing or inhibiting relapse of leukemia in a subject who has completed a cytotoxic treatment for leukemia.

In one embodiment, the agent selectively targets LRCs relative to HRCs. For example, in one embodiment the agent selectively reduces the level of LRCs in the subject relative to any reduction in the level of HRCs. In one embodiment, the agent that targets LRCs is an antagonist for a gene or protein encoded by a gene selected from VIPR2, PAFAH1B3, LPAR3, FGFR2, CLPS, KCNA4, BAAT, HTR4, NALCN, CARTPT, HTR1B, DRD2, BDKRB1, KCNJ10, SLC36A2, GRM5, KCNA10, SLC2A2 and PLG. In one embodiment, the agent that targets LRCs is a DRD2 antagonist, optionally thioridazine.

Also provided is a method for detecting Hematopoietic Regenerating Cells (HRCs) in a test sample. In one embodiment, the method comprises detecting a level of one or more biomarkers listed in Table 4C in the test sample and comparing the level of the one or more biomarkers in the test sample to one or more control levels. In one embodiment, the one or more control levels are representative of the level of the one or more biomarkers in HRCs and similarity between the level of the one or more biomarkers in the test sample and the one or more control levels is indicative of the presence of HRCs in the test sample.

Also provided are isolated populations of Leukemic Regenerating Cell (LRCs) as well as isolated populations of Hematopoietic Regenerating Cells (HRCs) as described herein. In one embodiment, the LRCs have increased expression of one or more biomarkers listed in Table 4A relative to a leukemic control sample that has not been exposed to cytotoxic treatment. In one embodiment, the HRCs have increased expression of one or more biomarkers listed in Table 4C relative to a healthy control sample not exposed to cytotoxic treatment.

Also provided are methods of screening test agents for use in preventing or inhibiting relapsing leukemia. In one embodiment, the method comprises contacting the test agent with LRCs as described herein and detecting a biological effect of the test agent on the LRCs. For example, in one embodiment a test agent that has the biological effect of reducing the level of LRCs is identified as a candidate for preventing or inhibiting relapsing leukemia. In one embodiment, the LRCs are in vitro.

In another embodiment, there is provided a method of screening a test agent for use in preventing or inhibiting relapsing leukemia by administering the test agent to a subject who has completed cytotoxic treatment for leukemia and determining a biological effect of the test agent on the subject. In one embodiment, the subject is a non-human animal, optionally a non-human transgenic animal comprising a leukemic xenograft.

### BRIEF DESCRIPTION OF THE DRAWINGS

Embodiments of the invention will now be described in relation to the drawings in which:

FIG. 1 shows human LSCs are not Selectively Spared After Repeated Chemotherapy Exposure. (A) BM cells were collected from a human AML patient, at diagnosis prior to therapy initiation (“−AraC”), and 7 days following the final dose of standard AraC-based induction therapy (“+AraC”). Fluorescence-activated cell sorting (FACS) plots show CD34 expression within mononuclear cells from patient BM aspirates. (B) FACS analysis of Hoechst/Pyronin Y cell cycle profiles within CD34+ AML cells before and after therapy. Pie charts show distribution of cell cycle phases (AML #1; as in A). (C) AML-xenografted mice were treated with AraC or vehicle control (“−AraC”). FACS plots show CD34 expression within human AML cells (CD45+CD33+) isolated from xenograft BM 24 hr after the final dose of AraC (AML #2). (D) Hoechst/Pyronin Y cell cycle profiles of CD34+ xenografted AML populations 24 hr after the final dose of AraC (as in C). Data points represent individual recipient mice. (E) BM cells were collected from a human AML patient before and after standard AraC-based induction therapy (as illustrated in A; AML #1). Leukemic cells were FACS-purified into CD34+ and CD34− subfractions, and evaluated in CFU progenitor assays. Data points represent individual CFU wells. (F) CD34+ and CD34− subfractions of human leukemic disease were FACS-purified from AML-xenograft BM and evaluated in CFU progenitor assays or serial transplantation assays (AML #2). Data points represent individual CFU wells (left) and individual secondary recipient mice (right). Symbols indicate the number of human leukemic cells transplanted per secondary recipient mouse (diamonds, 1×105; circles, 2.5×104). (G) BM cells collected from a human AML patient were evaluated in CFU progenitor assays before and after AraC-based induction therapy (AML #1). Data points represent individual CFU wells. Scale bar, 2 mm. (H-J) Human leukemic cells were recovered from the BM of primary recipient mice 48 hr after the final dose of AraC or vehicle control and analyzed in CFU progenitor assays (H) or serial transplantation assays (I-J). Data points represent individual CFU wells (H; scale bar, 2 mm) or individual secondary recipient mice (J). FACS plots show representative chimerism levels in the BM of secondary recipient mice transplanted with 2×105 human leukemic cells from primary xenograft BM (AML #2). Data are shown as mean±SEM. *p<0.05, **p<0.005, ***p<0.001, by unpaired t-tests (D,G,H,J), Mann-Whitney U test (D), or Fisher's Exact tests (E,F,H,J). Tests in D,G,H all compared −AraC vs. +AraC. See the STAR methods for further description of statistical tests used in D,H,J. See also FIG. 8, Tables 1 and 2.

FIG. 2 shows Cytoreductive Chemotherapy Fuels Accelerated Leukemic Regeneration. (A) Longitudinal profiling of human leukemic chimerism levels in the BM of AML-engrafted mice (AML #2 and #3), in response to AraC chemotherapy treatment in vivo (red bar, spanning 5 days). Curves represent individual mice, categorized based on residual disease levels post-AraC (<1% left; 1-5% middle, >5% right). Leukemic chimerism was defined as % human CD45+CD33+ within live BM cells. (B) Longitudinal profiling of leukemic blast percentages in the BM of a human patient (AML #4), in response to standard AraC-based induction chemotherapy (red bars). (C) Human leukemic chimerism levels in the BM of AML-engrafted mice over time, in response to in vivo AraC treatment. Leukemic chimerism was analyzed by FACS (% human CD45+CD33+) and is presented relative to pre-treatment levels. At each time examined, data points represent individual mice. (D) BM levels of healthy donor chimerism over time in response to in vivo AraC treatment. Human chimerism was analyzed by FACS (% human CD45+) and is presented relative to pre-treatment levels. At each time examined, data points represent individual mice. (E,F) Human leukemic (E) or healthy (F) chimerism levels were longitudinally monitored by serial BM aspirates of individual xenografted mice following treatment with AraC or vehicle control. Data points represent individual BM aspirate measures and curves represent group averages (left). Cellular growth rates were calculated for individual mice (right). Data are shown as mean±SEM. *p<0.05, **p<0.005, ***p<0.001 by one-way ANOVA with Newman-Keuls Multiple Comparison Test (C,D), or unpaired t-test (E). See also FIG. 9.

FIG. 3 shows recovery of Leukemia Initiating Cells and Progenitor Pools Precedes Disease Recurrence after Cytoreductive Chemotherapy. (A,B) Longitudinal analysis of BM leukemic chimerism levels (top), CD34+ frequencies within human AML populations (middle), and leukemic progenitor content within human AML populations (bottom) over time in response to in vivo AraC treatment (red bars, spanning 5 days). Independent experiments represent surrogate states of residual disease <5% (A) or >5% (B). n=6 xenografted mice per AML patient sample (top and middle). Data points represent individual CFU wells (bottom). Grey bars indicate the onset of regeneration, marked as the transition point prior to disease re-growth (top). (C) Xenografts derived from 3 distinct AML patient samples were evaluated by FACS, in CFU progenitor assays, and in limiting dilution serial transplantation assays at the defined onset of regeneration as determined in (A,B). n=4-12 primary recipient mice for FACS analysis, n=3-7 CFU wells, n=9-43 secondary recipient mice per sample. Data are shown as mean±SEM. See also FIG. 9 and Table 3.

FIG. 4 shows molecular Signatures of Leukemic Regeneration are Distinct from Therapy-naive AML and Healthy Regeneration. (A) Experimental overview for (B-D). Human AML cells were FACS-purified from the BM of xenografted mice for CFU progenitor assays and global gene expression profiling at the defined onset of leukemic regeneration, 9 days following the final dose of AraC (“+AraC”) or vehicle control (“−AraC”). (B) Leukemic progenitor numbers within human AML populations purified from xenograft BM. Data points represent individual CFU wells. **p<0.005, ***p<0.0001, by unpaired t-tests. (C) GSEA plot comparing genes associated with therapy-naive LSCs (Eppert et al., 2011) in AraC-exposed human AML vs. vehicle controls (“−AraC”). n=4 xenografts per treatment group, derived from 3 distinct AML patient samples (#2, #5, and #7). NES, normalized enrichment score. (D) STRING network representation of the highest confidence protein-protein interactions among genes that were upregulated in AraC-treated human AML vs. vehicle controls (Table 4). Shaded nodes indicate genes associated with GPCR signaling (GO.0007186, GO.0007187, GO.0007205, GO.0007200, or GO.0007188). (E) Experimental overview for (F and G). Healthy hematopoietic cells were FACS-purified from the BM of xenografted mice for CFU progenitor assays and global gene expression profiling, 9 days following the final dose of AraC (“+AraC”) or vehicle control (“−AraC”). (F) Progenitor numbers within healthy human hematopoietic populations, purified from xenograft BM. Data points represent individual CFU wells. (G) STRING network representation of the highest confidence protein-protein interactions among genes that were upregulated in AraC-treated healthy human hematopoietic cells vs. vehicle controls (Table 4). Shaded nodes indicate genes associated with hematopoietic differentiation (GO.004563 or GO.1903706), or response to stress (GO.0006950). (H) Venn diagram illustrating overlap between protein-coding genes that are upregulated in regenerating human AML cells versus regenerating healthy human cells (Table 4). (I) Heat map showing 19 druggable up-regulated genes unique to leukemic regeneration signatures identified in (H). Data are shown as mean±SEM. See also FIG. 10 and Table 4.

FIG. 5 shows transient Features of Leukemic Regeneration are Mediated by Cell-extrinsic Factors. (A) Experimental overview for (B). Human AML cells were cultured in vitro in IMDM media with 20% normal mouse serum, containing 0.15 μM or 1.0 μM AraC, or 0.1% DMSO (“−AraC”). After a 24 hr culture, CFU progenitor assays were performed. (B) Leukemic progenitor numbers after direct in vitro incubation with AraC. Values are normalized relative to DMSO vehicle control cultures. Data points represent individual CFU wells. (C) Experimental overview for (D-F). Primary AML samples were cultured in IMDM containing 20% serum obtained from NOD/SCID mice recovering from AraC cytoreduction (“AraC serum”), or from vehicle-treated control mice (“control serum”). After a 24 hr culture, CFU progenitor assays and FACS analyses were performed. (D) Leukemic progenitor numbers after culture in AraC serum or control serum. Data points represent individual CFU wells. For each patient, cultures were performed with at least 3 biologically independent serum samples per condition. (E,F) FACS histograms and fold change of SLC2A2 (E) and DRD2 (F) protein expression after exposure to AraC serum or control serum (as in C). Replicate data points per patient were cultured in biologically independent serum collections. Data are shown as mean±SEM. **p<0.005, ***p<0.001 by one-way ANOVA with Newman-Keuls Multiple Comparison Test (B) or unpaired t-tests (D,F), or Mann-Whitney U test (E). See also FIG. 11 and Table 5.

FIG. 6 shows LRC Targeting Interrupts Aggressive AML Re-growth Following AraC Treatment. (A) AML-engrafted mice were treated with DRD2 antagonist (thioridazine) or vehicle control (“−DRD2 antagonist”) as a single agent in the absence of AraC (i.e., targeting therapy-naive LSCs). At the completion of treatment, human AML cells were purified from xenograft BM 1 day following a 21-day treatment regimen, and evaluated in CFU progenitor assays. Data points represent individual CFU wells. (B) AML-engrafted mice were treated with DRD2 antagonist (thioridazine) or vehicle control in combination with AraC chemotherapy (i.e., targeting LRCs). Human AML cells were purified from xenograft BM 9 days after the last dose of AraC and were evaluated in CFU progenitor assays. DRD2 antagonist treatment was continued until the day before primary recipient BM was harvested. Data points represent individual CFU wells. (C) AML-engrafted mice were treated with DRD2 antagonist or vehicle control in combination with AraC chemotherapy. Leukemic chimerism levels were analyzed by FACS 9 days following the final dose of AraC (as in B). Data points represent individual mice. (D) AML-engrafted mice were treated with DRD2 antagonist or vehicle control in combination with AraC chemotherapy. Disease regeneration was monitored by serial BM aspirates, starting from the onset of leukemic regeneration post-AraC (arrow). Curves represent individual mice. (E) Cellular growth rates were calculated for individual AML-engrafted mice based on serial BM aspirates in (D). Data points represent individual mice. (F) AML-engrafted mice were treated with DRD2 antagonist or vehicle control in combination with AraC chemotherapy. AML cells were collected from primary xenograft BM 9 days following the final dose of AraC (as in B), for serial transplantation and CFU progenitor assays. Data points represent individual CFU wells or individual mice. Data are shown as mean±SEM. *p<0.05, **p<0.01 by unpaired t-tests (A,B,E) or Fisher's Exact Tests (C,F). See also FIG. 13 and Table 6.

FIG. 7 shows signatures of Leukemic Regeneration are Detected in Human AML Patients Post-Chemotherapy. (A) Experimental overview for (B-G). Human AML cells were recovered from patients at diagnosis (“untreated”), or at the point of cytoreduction approximately 3 weeks following AraC-based chemotherapy treatment (“+AraC”), and evaluated by CFU progenitor assays, global gene expression profiling, and FACS analysis. (B) Progenitor numbers in leukemic populations recovered directly from AML patients. Data points represent individual CFU wells. (C) GSEA plot comparing genes associated with therapy-naive LSCs (17-gene signature) (Ng et al., 2016) in leukemic populations recovered from patients after clinical chemotherapy treatment (“+AraC”) vs. at diagnosis before chemotherapy exposure (“untreated”). n=4 patients. NES, normalized enrichment score. (D) FACS plot showing gating strategy and quantification of CD34 expression within leukemic populations recovered directly from AML patients. (n=6 individual patients; #11, #16-20). (E) Mean fluorescence intensity (MFI) of candidate LRC proteins within leukemic populations recovered directly from AML patients. Analysis was performed within leukemic blast gates as shown in (D). (F) FACS histograms showing LRC protein expression within CD34+ leukemic populations. (G) Hierarchical clustering of 182 LRC-specific protein-coding genes in AraC-exposed human AML cells recovered from clinically treated patients (#11, #15-17) or AraC-treated xenografts (#2, #5, #7). “−AraC” controls represent untreated cells obtained from patients at diagnosis, or from vehicle-treated xenografts. AML patient IDs are shown under human or mouse silhouettes. (H) Expression levels of LRC genes within human leukemic cells obtained from AML patients during regenerative phases post-chemotherapy (n=4 patients; #11, #15-17), or at relapse (n=15 patients; #21-24 and GSE66525). Expression values are normalized to matched diagnosis samples from the same patients (set to 1.0). Data points represent individual genes, averaged across patients. (I) Experimental overview for (J-L). BM cells were recovered from patients during states of remission (hollow silhouettes) or refractory leukemic disease (solid silhouettes; AML #11, #16-20) approximately 3 weeks following AraC-based chemotherapy treatment. Within remission cases, patients either maintained durable healthy remission states (>5 years; AML #25-27) or developed relapse within 6-13 months (AML #21, 23, 28, 29). (J) Fold change in % SLC2A2 expression within CD34+ cells obtained from patient BM post-chemotherapy treatment. Expression levels are normalized to “untreated” control cells obtained at diagnosis (set to 1.0). Data points represent individual patients. (K) SLC2A2 expression levels within CD34+ cells from AML patient BM at remission. Data points represent individual patients (IDs are indicated next to data points). (L) CD34+ cells were FACS-purified from AML patient BM based on SLC2A2 expression, during states of clinical remission prior to relapse. DNA was extracted from purified cell fractions, and droplet digital PCR was performed to quantify the abundance of patient-specific NPM1 aberrations. Values are expressed relative to bulk AML cells (“unsorted”). Data are shown as mean±SEM. *p<0.05, **p≤0.001, *** p<0.0001 by unpaired t-tests (B,J,K), paired t-test (D), or Mann-Whitney U test (H). See also FIG. 12.

FIG. 8 shows cellular Responses to Cytoreductive Chemotherapy are Shared Between Human Patients and AML-Xenografts. (A) Experimental overview for (B-E). BM cells were collected from a human AML patient (AML #1), at initial diagnosis prior to therapy initiation (−AraC), and 7 days following the final dose of AraC-based induction therapy (+AraC). (B) BM smear images showing that patient BM remained predominantly leukemic after therapy. Scale bar, 10 pm. (C) FACS plot showing that patient BM remained predominantly leukemic in composition after therapy (upper panel) and clinically-measured circulating blast counts (lower panel). (D) FAGS analysis of Hoechst/Pyronin Y cell cycle profiles within bulk AML cells or subfractions obtained from patient BM. (E) FACS plots showing CD34+CD38− expression profiles in AML patient BM cells. (F) Total WBC counts were measured daily in n=6 individual AML patients during and subsequent to treatment with AraC-based induction therapy. (G) BM cellularity and white blood cell (WBC) counts were monitored in NOD/SCID mice over time, following in vivo treatment with AraC. Per time point, n=4-19 mice for the analysis of BM cellularity and n=2-12 mice for WBC counts. (H) Experimental overview for (1-0) AML-engrafted mice were treated with AraC or vehicle control (“−AraC”) in vivo. Human AML cells were recovered from xenograft BM for cell surface profiling and cell cycle analysis by FACS. (1) FACS plots showing CD341−CD38− expression profiles within human AML populations recovered from xenograft BM 48 hr after the last dose of AraC in vivo (as in H). Plots are representative of independent experiments performed with AML #2 (left, low CD34 content), and AML #3 (right, high CD34 content). (J) CD34 expression within human AML populations recovered from xenograft BM 24-72 hr after the final dose of AraC. Per time point, n=5 mice per group (AML #2) and n=4-5 mice per group (AML #3). (K) CD34′CD38− expression within human AML populations recovered from xenograft BM 24-72 hr after the final dose of AraC. Per time point, n=5 mice per group (AML #2) and n=4-5 mice per group (AML #3). (L) Fold reduction in total leukemic cell numbers (left), total CD34+ leukemic cell numbers, and total CD34− leukemic cell numbers (right). Cells were recovered from the BM of AML-xenografts 48 hr following the final dose of AraC treatment. Values are expressed relative to vehicle controls. n=6 AraC-treated mice per group. (M) Frequency of quiescent GO cells within CD34+ human AML cells recovered from xenograft BM. GO populations were quantified by Hoechst/Pyronin Y FACS analysis. n=5 mice per group. (N) FACS plots and analysis showing Ki67 expression within CD34+ human AML cells recovered from xenograft BM. n=5 mice per group (AML #2). (0) FACS plots and analysis showing BrdU levels within CD34+ human AML cells recovered from xenograft BM. n=5 mice per group (AML #2) and n=4-5 mice per group (AML #3). (P) AML-xenografts were FACS purified into CD34+ and CD34− fractions, and evaluated in CFU progenitor assays (AML #3). Data points represent individual CFU wells. (Q) Absolute number of leukemia initiating cells in primary recipient mice engrafted with human AML (#2 and #3). Cells were serially transplanted 48 hr after the last dose of AraC or vehicle control (“−AraC”, as in H). Data correspond to FIG. 1J. Data are shown as mean±SEM. *p<0.05, **p<0.01, *“p<0.001 by one-way ANOVA with Newman-Keuls Multiple Comparison test (J,K,N,0), Kruskal-Wallis Test with Dunn's Multiple Comparison Test (0), unpaired t-test (P), or Mann-Whitney U tests (L).

FIG. 9 shows human AML Disease Rapidly Regenerates Following Cytoreductive Chemotherapy in Xenografts. (A) Human leukemic chimerism levels were longitudinally monitored by serial BM aspirates of individual AML-engrafted mice following treatment with AraC (red bar, spanning 5 days) or vehicle control (AML #2). Data points represent individual BM aspirate measures and curves represent group averages (left). Cellular growth rates calculated for individual mice (right). (B, C) Serial BM aspirates and cellular growth rates calculated by excluding the final data point for vehicle-control mice (“−AraC”) when BM becomes saturated with disease. Analyses correspond with full data sets presented in FIG. 2E (B), and FIG. 9A (C). (D, E) Serial BM aspirates of representative mice from each treatment group, matched based on initial disease levels (left). Time scales are shifted to superimpose leukemic growth curves (right). Analyses correspond with full data sets presented in FIG. 2E (D) and FIG. 9A (E). (F) Human leukemic chimerism levels were longitudinally monitored by serial BM aspirates of individual AML-engrafted mice following treatment with either 50 mg/kg or 100 mg/kg AraC (red bar, spanning 5 days). Dots represent individual xenografted mice (AML #2) and curves represent group averages. Data are shown as mean±SEM. *p<0.05, **p<0.005, ***p<0.0001 (unpaired t-tests).

FIG. 10 shows In vitro Progenitor Assays Predict Functional Performance in Leukemia-Initiating Assays. Human leukemic populations were isolated from AML-xenografts and leukemia-initiating capacity was measured by serial transplantation at limiting dilution, in parallel with measures of colony formation potential in CFU progenitor assays. Each data point represents the group average of an individual experiment. Values represent functional cell frequencies among AML populations recovered from the BM of AraC-treated xenografts measured during cytoreductive periods post-AraC (2 days after the final AraC dose) or at the onset of regeneration (9 days after the final AraC dose). Values are normalized to vehicle-treated controls. AML patient IDs are indicated by the numbers inside each data point. *p<0.05 (Pearson's correlation). See also Tables 2 and 3.

FIG. 11 shows Molecular Profiles Distinguish LRCs from Therapy-Naive AML. (A) GSEA plot showing a gene set representing therapy-naive LSCs (Eppert et al., 2011), applied to gene expression profiles from de novo AML patient samples that generate human leukemic grafts in mice (engrafters) vs. AML patient samples that lack leukemic reconstitution capacity (non-engrafters). (B) Experimental overview for (C and D). Human AML cells were recovered from xenograft BM, 9 days following the final dose of AraC or vehicle control (“−AraC”). Candidate LRC markers (C) and cell cycle profiles (D) were measured by FACS analysis. (C) FACS histograms and quantified mean fluorescence intensity (MFI) values of candidate LRC proteins gated within human CD45+CD33+ leukemic populations from AML-xenograft BM (AML #3). Data points represent individual mice. (D) Hoechst/Pyronin Y cell cycle profiles within CD34+ xenografted AML populations (AML #3). Plots are representative of n=4 xenografted mice per group, not significant. Data are shown as mean±SEM. **p<0.01, ***p<0.0001 (unpaired t-test).

FIG. 12 shows Features of Leukemic Regeneration are not Recapitulated Ex Vivo. (A) Whole genome sequencing was performed on human AML cells that were FACS-purified from a primary patient sample (AML #2) and from the BM of AraC-treated xenografts derived from the same patient. Cells were recovered from mice at the point of disease recurrence after AraC therapy in vivo. Tumor-specific mutations were identified using healthy T cells purified from the same patient. Scatter plot shows genome-wide variant allele frequencies of high-confidence SNVs detected in primary human patient cells (x axis) vs. a matched AraC-treated xenograft (y axis). SNVs occurring in known myeloid cancer genes (Papaemmanuil et al., 2016) are labeled. Colors indicate clusters of co-varying SNVs, with the number of SNVs per cluster indicated in the legend (brackets). Xenograft data are representative of n=4 individual mice. (B) Experimental overview for (C and D). Human AML cells were cultured in vitro in growth medium containing 0.15 pM or 1.0 pM AraC, or 0.1% DMSO (“−AraC”) for 5 consecutive days, followed by continued culturing in the absence of drug treatment. At 1-2 day intervals, viable cell counts were measured (C) and CFU progenitor assays were performed (D). (C) Viable leukemic cell counts were measured using the MACSQuant Analyzer system, and normalized to viable cell numbers plated per well on Day 0. Arrowheads indicate AraC treatment days. n=6-8 wells each (AML #3 and #8). (D) Leukemic progenitor numbers within human AML populations cultured with AraC or DMSO control. At each time point, values are normalized to vehicle control. Arrowheads indicate AraC treatment days. n=6-9 wells each (AML #3, #8, and #12). (E) Human AML cells were cultured in vitro in growth medium containing 0.15 pM or 1.0 pM AraC, or 0.1% DMSO (“−AraC”) for 5 consecutive days, followed by extended culturing in the absence of drug treatment. FACS plots show viability measured by 7AAD exclusion, measured at Day 16 of culture. (F) Human AML cells were cultured in vitro in growth medium containing 0.15 pM or 1.0 pM AraC, or 0.1% DMSO (“−AraC”) for 5 consecutive days, followed by continued culturing in the absence of drug treatment. At Day 8 of culture, candidate LRC proteins were quantified by FACS (gated on live cells; AML #8). (G) Human AML cells were cultured in vitro in IMDM media with 20% normal mouse serum, containing 0.15 pM or 1.0 pM AraC, or 0.1% DMSO (“−AraC”). After 24 hr of culture, candidate LRC proteins were quantified by FACS (gated on live cells). Protein expression levels are shown normalized to vehicle control. Data points represent individual cell culture wells. Data are shown as mean±SEM. ***p<0.0001 (two-way ANOVAs). See also Table 5.

FIG. 13 is related to FIG. 6 and shows that DRD2 Antagonist Treatment Achieves Clinically Relevant Plasma Levels in vivo, and Counteracts AraC-Mediated LRC Features. (A) Plasma concentrations of DRD2 antagonist TDZ, measured 3 hr after a 21-day administration in NOD/SCID mice. The 22.5 mg/kg dose was selected for subsequent in vivo analyses, as it attained clinically relevant ranges of plasma TDZ (200-2000 ng/ml). Data points represent individual mice. (B) Plasma concentrations of DRD2 antagonist TDZ after a single administration at 22.5 mg/kg in NOD/SCID mice. n=3 mice per time point. (C) Viable BM cell counts (left) and H&E-stained BM sections (right) the day after treatment of NOD/SCID mice with vehicle control or DRD2 antagonist for 21 days. Data points represent individual mice. Scale bar, 30 μm. (D) White blood cell (WBC) counts the day after treatment of NOD/SCID mice with vehicle control or DRD2 antagonist (22.5 mg/kg/day) for 21 days. Data points represent individual mice. (E) AML-engrafted mice were treated with DRD2 antagonist TDZ or vehicle control. DRD2 antagonist was delivered either as a single agent (i.e., targeting therapy-naive LSCs) or together with AraC (i.e., targeting LRCs). Human AML cells were purified from xenograft BM 9 days following the final dose of AraC, and analyzed in CFU progenitor assays. DRD2 antagonist treatment was continued until the day before analysis. Total numbers of leukemic progenitors per mouse were estimated based on CFU counts per human AML cells plated, multiplied by human AML cellularity in mouse BM (AML #5). Each data point is derived from an independent CFU well. (F) AML-engrafted mice were treated with DRD2 antagonist TDZ or vehicle control. DRD2 antagonist was delivered either as a single agent (i.e., targeting therapy-naive LSCs) or together with AraC (i.e., targeting LRCs). Leukemia initiating cell frequencies were estimated by serial transplantation at limiting dilution at the time of LRC emergence 9 days post-AraC treatment (AML #5; n=4-26 secondary transplant recipients per condition). DRD2 antagonist treatment was continued until the day before harvesting primary recipient BM. (G) Kaplan Meier analysis of relapse-free survival in AML-engrafted mice after in vivo exposure to AraC plus TDZ (“LRCs+DRD2 antagonist”) vs. AraC alone (“LRCs+vehicle”). Time to relapse was defined for individual mice based on the time from initial cytoreduction to overt disease recurrence (set at 20% leukemic chimerism), as estimated using AML growth rates (AML #3, FIG. 2E). p=0.08 (Mantel-Cox test). n=4-6 mice per group. (H) DRD2 protein mean fluorescence intensity (MFI) within human leukemic populations recovered from xenograft BM after exposure to AraC versus DRD2 antagonist in vivo. Xenografts were derived from 3 AML patients (diamonds, AML #5; circles, AML #6; squares, AML #14). DRD2 protein levels are expressed relative to matched vehicle-treated controls (“therapy-naive”; dotted line). Data points represent individual xenograft recipients. (I) FACS histograms showing DRD2 protein expression within human leukemic populations recovered from xenograft BM at the LRC stage post-AraC, with or without in vivo exposure to DRD2 antagonist treatment (AML #6 and #14). DRD2 antagonist treatment was continued until the day before analysis. Data are shown as mean±SEM. *p<0.05, **p<0.01, ***p<0.001 by one-way ANOVA with Newman-Keuls Multiple Comparison Test (E), ELDA goodness of fit test (F), or unpaired t-test (H).

FIG. 14 is related to FIG. 7 and shows Patterns of Leukemic Regeneration are Conserved Between AraC-Treated Xenografts and Clinically-Treated AML Patients. (A) Experimental overview for (B-F). Human AML cells were recovered from patients at diagnosis (“untreated”), or approximately 3 weeks following AraC-based chemotherapy treatment (“+AraC”). (B) Clinically-measured circulating blast counts and (C) Leukemic blast percentages in paired diagnosis (“untreated”) vs. post-therapy (“+AraC”) samples used for gene expression and CFU progenitor analyses (FIGS. 7B and 7C). (D) BM smear images obtained from a human AML patient (AML #17). Scale bar, 10 pm. (E) GSEA plot showing a gene set representing therapy-naive LSCs (Eppert et al., 2011), applied to gene expression profiles from AML patient samples shown in (A-D). n=4 matched diagnosis (“untreated”) vs. post-therapy (“+AraC”) patient samples. (F) GSEA plot showing gene sets representing the 182-gene LRC signature vs. an AML chemoresistance signature (Farge et al. 2017), applied to gene expression profiles from AML patient samples shown in (A-D). n=4 matched diagnosis (“untreated”) vs. post-therapy (“+AraC”) patient samples. (G) Experimental overview for (H and I). BM was collected from AML-xenografts (AML #2), following treatment with AraC (“+AraC”) or vehicle control (“−AraC”) for FACS analysis. +AraC conditions represent regenerative time points of LRCs, which is 9 days following the final dose in xenografts. (H) CD34 expression within human AML populations recovered from xenograft BM. n=4 xenografts per condition. (I) FACS histograms showing the expression of candidate LRC proteins within CD34+ human leukemic subsets recovered from xenograft BM. (J) Longitudinal monitoring of candidate LRC protein expression within CD34+ leukemic BM cells obtained from AML Patient #11. BM samples were obtained at diagnosis (prior to exposure to AraC-based therapy), as well as at the point of cytoreduction post-AraC and subsequent regeneration (LRC). Note that timelines of therapy response are longer in patients than in xenografts, as outlined in FIGS. 8F and 8G. MFI, mean florescence intensity. (K) GSEA plot showing 182 LRC-specific genes, applied to gene expression profiles obtained from AraC-exposed AML-xenografts during initial cytoreductive periods 72 hr post-treatment (GSE97631; Farge et al. 2017). n=3 xenografts per condition. (L) Longitudinal profiling of human leukemic chimerism levels in the BM of AML-engrafted mice, in response to multiple rounds of AraC chemotherapy treatment in vivo (red bars, spanning 5 days each) (left). FACS histograms show LRC marker expression within CD34+ human leukemic populations recovered from xenograft BM at “relapse”, or subsequent to a second round of AraC treatment (“re-induced LRC”) (right). Each curve represents a primary xenograft recipient. (M) FACS plots showing co-expression of LRC markers DRD2 and SLC2A2 in leukemic cells recovered from the BM of AML patient #11 at regenerative stages post-chemotherapy (−3 weeks following the completion of induction therapy). NES, normalized enrichment score. Data are shown as mean±SEM. ***p<0.0005 (unpaired t-test).

### DETAILED DESCRIPTION OF THE INVENTION

The present description provides methods for determining a prognosis for a subject with leukemia. Remarkably, relapse of subjects having undergone chemotherapy for AML has been shown to be associated with the presence of cells termed Leukemic Regenerating Cells (LRCs) that are readily distinguished from healthy leukocytes or therapy-naive leukemic stem cells. A separate but corresponding population of Hematopoietic Regenerating Cells (HRCs) have been shown to emerge following the administration of the cytotoxic agent cytarabine to subjects without leukemia as well as in response to 5-fluoruracil or radiation. The present description also provides methods for the treatment of leukemia that target the emergence of LRCs following cytotoxic treatment to reduce the likelihood of relapsing disease as well as screening methods to identify agents useful for preventing or inhibiting the relapse of leukemic disease. In one embodiment, the leukemia is acute myeloid leukemia (AML).

In one embodiment, there is provided a method of determining a prognosis for a subject who has completed a cytotoxic treatment for leukemia. In one embodiment, the method comprises:

determining a level of one or more biomarkers listed in Table 4A in a test sample obtained from the subject following the cytotoxic treatment for leukemia; and

comparing the level of the one or more biomarkers in the test sample to one or more control levels,

wherein a difference or similarity in the level of the one or more biomarkers in the test sample compared to the one or more control levels is indicative of whether the subject has an increased or decreased risk of relapsing leukemia.

Also provided is a method of detecting Leukemic Regenerating Cells (LRCs) in a test sample. In one embodiment, the method comprises:

determining a level of one or more biomarkers listed in Table 4A in the test sample; and

comparing the level of the one or more biomarkers in the test sample to one or more control levels.

In one embodiment, the one or more control levels are representative of the level of the one or more biomarkers in LRCs and similarity between the level of the one or more biomarkers in the test sample and the one or more control levels is indicative of the presence of LRCs in the test sample. Optionally, the test sample is in vivo, ex vivo or in vitro.

As used herein a “biomarker” refers to a biomolecule such as a nucleic acid, protein or protein fragment present in a biological sample from a subject, wherein the quantity, concentration or activity of the biomarker in the biological sample provides information about whether the subject has, or is at risk of developing, relapsing acute myeloid leukemia. In one embodiment, the biomarker(s) described herein are useful for identifying whether a cell is a LRC.

The term “leukemia” as used herein refers to any disease involving the progressive proliferation of abnormal leukocytes found in hemopoietic tissues, other organs and usually in the blood in increased numbers. “Leukemic cells” refers to leukocytes characterized by an increased abnormal proliferation of cells. Leukemic cells may be obtained from a subject diagnosed with leukemia.

The term “acute myeloid leukemia” or “acute myelogenous leukemia” (“AML”) refers to a cancer of the myeloid line of blood cells, characterized by the rapid growth of abnormal white blood cells that accumulate in the bone marrow and interfere with the production of normal blood cells.

As used herein, “relapsing leukemia” or “recurrent leukemia” refers to a disease state associated with a complete or partial remission in response to treatment followed by the recurrence of leukemia.

The term “subject” as used herein refers to any member of the animal kingdom. In one embodiment, the subject is a mammal, such as a human. In one embodiment, the subject is a human presenting with AML or suspected of having AML.

The term “determining a prognosis” refers to a prediction of the likely progress and/or outcome of an illness, which optionally includes defined outcomes such as risk of relapsing disease. In some embodiments, determining a prognosis may involve a binary classification such as classifying a subject as having a high risk or a low risk of relapsing AML. In some embodiments, determining a prognosis may involve calculating a quantitative risk score, wherein the magnitude of the risk score is indicative of the risk of a subject having or developing relapsing AML.

As used herein the term “control level” refers to a level of a biomarker in a comparative sample or a pre-determined value associated with a known disease state or outcome. A “control level” may also be a level of a biomarker associated with or representative of a control sample. In one embodiment, the control level is representative of normal, disease-free cells, tissue, or blood. In one embodiment, the control level is representative of subjects with cancer for whom the clinical outcome of the disease is known. For example, in one embodiment the control level is representative of subjects who have, or develop, relapsing leukemia, optionally relapsing AML. Alternatively, the control level may be representative of subjects who do not have or develop relapsing leukemia. In one embodiment, the control level is representative of the level of a biomarker in LRCs. Alternatively, the control level may be representative of the level of a biomarker in cells that are not LRCs such as healthy hematopoietic cells, optionally HRCs. In one embodiment, the control level is a level of expression of a biomarker in therapy naïve leukemic cells obtained at diagnosis from a subject for whom the prognosis is being determined.

Table 4A identifies a number of biomarkers useful for the identification of LRCs and reports expression levels in AraC-exposed LRCs vs. non-treated AML cells. The biomarker data contained herein can be used individually or in combination to generate biomarker expression profiles indicative of LRCs relative to other types of cells. In one embodiment, the one or more biomarkers comprise biomarkers selected from FASLG, DRD2, SLC2A2, and FUT3.

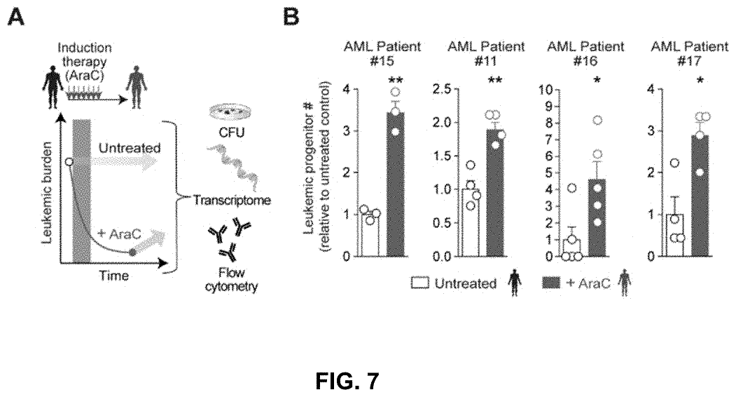

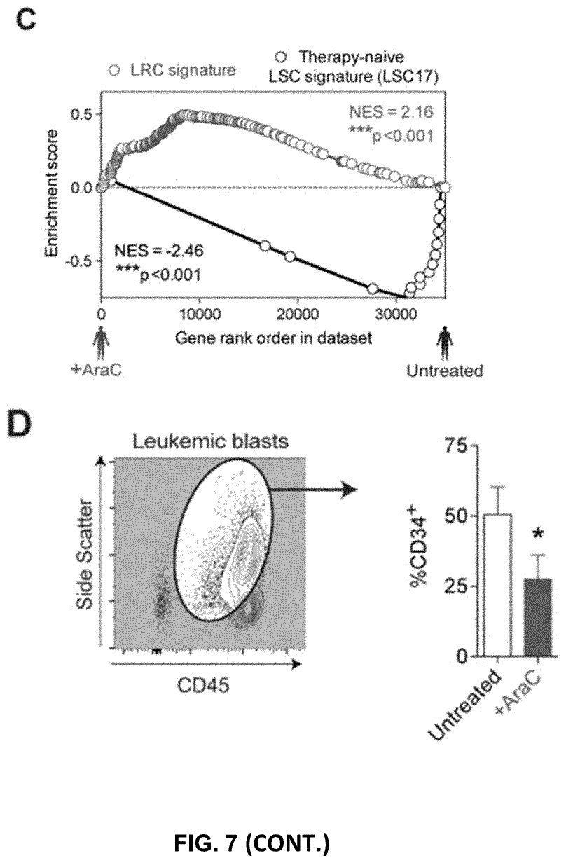

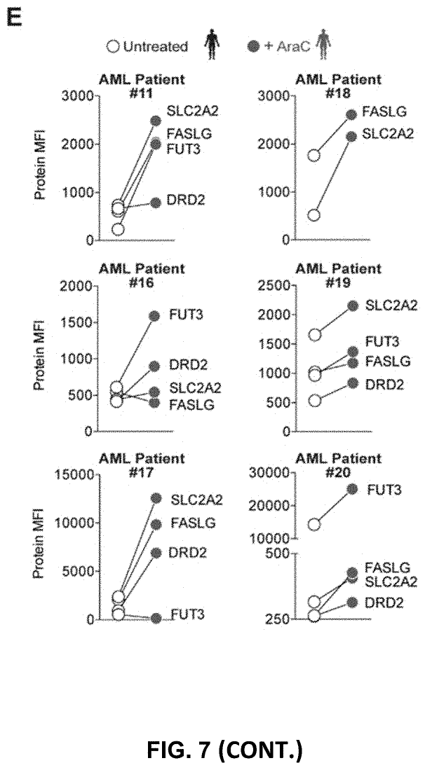

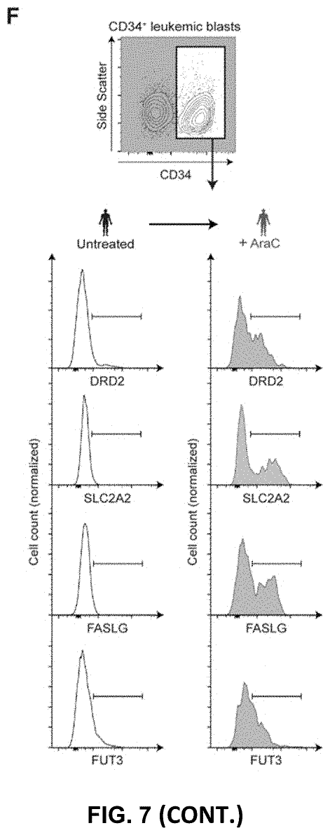

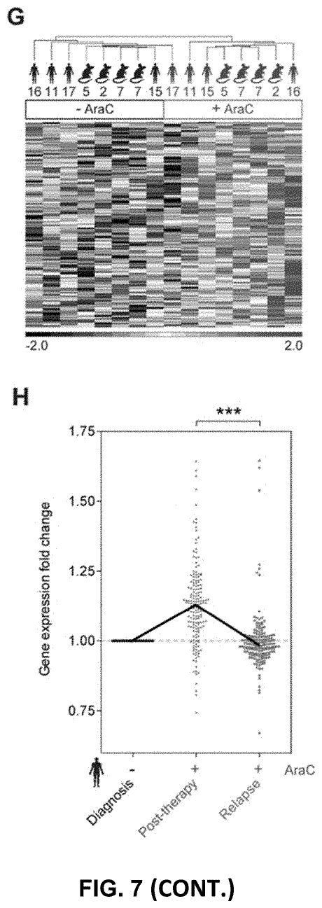

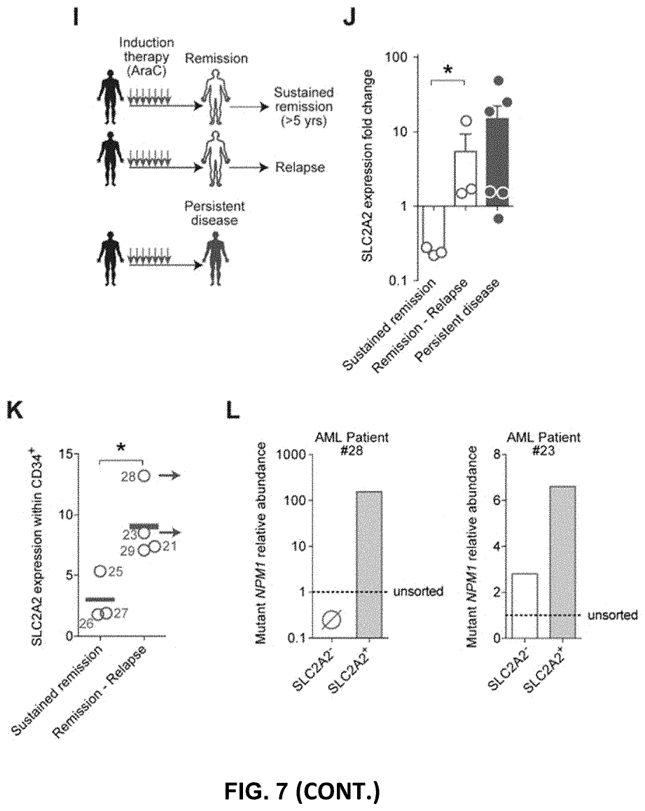

As shown in FIG. 7, SLC2A2 expression at remission stratified discriminated between subjects with sustained remission versus eventual relapse. In one embodiment, the method comprises determining a level of SLC2A2 in the test sample wherein an increased level of SLC2A2 in the test sample compared to the control level is indicative of an increased risk of relapsing AML.

A number of biomarkers were also identified whose expression was reduced or absent in LRCs relative to other cells. For example, in one embodiment the method comprises determining a level of ANGPT1 and/or HMOX1 in the test sample, wherein a reduced level of ANGPT1 and/or HOX1 in the test sample compared to the control level(s) is indicative of an increased risk of relapsing AML.

In one embodiment, the methods described herein include comparing the level of one or more biomarkers in a test sample to a level of one of more biomarkers in a control sample. The term “sample” as used herein refers to any fluid or other specimen from a subject that can be assayed for biomarker levels, for example, blood, serum, plasma, saliva, cerebrospinal fluid or urine. In one embodiment, the sample is whole blood, a fractionated blood sample or a bone marrow sample. In one embodiment, the test sample comprises mononuclear cells. In one embodiment, the test sample comprises leukemic cells, optionally AML cells. In one embodiment, the test sample comprises CD45+ cells. In one embodiment, the test sample comprises CD34+ cells, or CD34+ and CD38− cells.

The term “level” as used herein refers to the quantity, concentration, or activity of a biomarker in a sample from a subject. In one embodiment, the biomarker is a protein or protein fragment and the biomarker is detected using methods known in the art for detecting proteins such as, flow cytometry, ELISA or mass spectroscopy. In one embodiment, the biomarker is a protein or mRNA and the level is an expression level of the corresponding protein or mRNA. Optionally, the biomarker is an enzyme and enzyme activity levels are determined in a test sample from a subject to indicate a level of the biomarker in the subject.

In one embodiment, the one or more biomarker levels in the test sample are compared to levels of one or more biomarkers in a control sample. Optionally, the phrase “level of one or more biomarkers in a control sample” refers to a predetermined value or threshold of a biomarker or levels or more than one biomarker, such as a level or levels known to be useful for identifying subjects having, or at risk of developing, relapsing leukemia.

In some embodiments, the methods described herein comprise determining the level of one or more biomarkers in a test sample. Optionally, determining the level of one or more biomarkers in the test sample comprises detecting a nucleic acid molecule or polypeptide encoding for all or part of the biomarker. Various methods known in the art may be used to test for and detect the level of a biomarker in test sample as described herein. For example, in one embodiment, detecting the level of one or more biomarkers in the test sample comprises contacting the sample with a binding agent selective for the biomarker. In one embodiment, detecting the level of the biomarkers comprises the use of flow cytometry and/or FACS. In one embodiment, detecting the level of the biomarkers comprises using Nanostring, flow cytometry, microscopic imaging, microarray chip, PCR and/or RT-PCR.

Comparing the level of one or more biomarkers in a test sample to one or more control levels can be performed by a number of different methods or techniques known in the art. For example, in one embodiment the levels of individual biomarkers, such as those listed in Table 4A, are compared to determine if there is a difference indicative of the subject having, or at risk of developing, relapsing leukemia. As set out in the Examples, molecular signatures associated with LRCs cans be used to identify subjects having a greater risk of relapsing disease.

For example, the level can be a concentration such as μg/L or a relative amount such as 1.2, 1.3, 1.4, 1.5, 1.6, 1.7, 1.8, 1.9, 2.0, 2.2, 2.4, 2.6, 2.8, 3.0, 3.2, 3.4, 3.6, 3.8, 4.0, 4.2, 4.4, 4.6, 4.8, 5.0, 10, 15, 20, 25, 30, 40, 60, 80 and/or 100 times or greater a control level, standard or reference level. Optionally, a control is a level such as the average or median level in a control sample. The level of biomarker can be, for example, the level of protein, or of an mRNA encoding for the biomarker such as SLC2A2.

In another embodiment, levels of more than one biomarker are compared to determine a prognosis for a subject with leukemia, optionally by generating a biomarker expression profile and comparing the biomarker expression profile with a control profile. Methods that can be used to compare biomarker levels in a test sample and control levels include, but are not limited to, analysis of variance (ANOVA), multivariate linear or quadratic discriminant analysis, multivariate canonical discriminant analysis, a receiver operator characteristics (ROC) analysis, and/or a statistical plots. In one embodiment, comparing the biomarker expression profiles comprises multivariate analysis. Machine learning methods may also be used to compare biomarker expression profiles in order to determine a prognosis and e.g. classify a test sample as comprising LRCs and identifying the test subject as having an increased risk of relapsing AML. Techniques such as Gene Set Enrichment Analysis (GSEA) and variants thereof may also be used to compare biomarker expression profiles. Method of comparing biomarker expression profiles may also be used for detecting LRCs and/or HRCs in a test sample as described herein.

In one embodiment the control biomarker expression profile is representative of LRCs and a similarity in the biomarker expression profile of the test sample and the control biomarker expression profile is indicative of an increased risk of relapsing leukemia.

In one embodiment, the methods described involve calculating a risk score for the subject based on a difference or similarity in the biomarker expression profile of the test sample and the control biomarker expression profile. In one embodiment, the magnitude of the risk score is indicative of relapsing leukemia in the subject.

Optionally, the subject may be classified as having a good prognosis and a low risk of relapsing leukemia if the subject risk score is low and/or below a selected threshold or as having a poor prognosis and a high risk of relapsing leukemia if the subject risk score is high and/or above the selected threshold.

In one embodiment, there is also provided a computer-implemented method for determining a prognosis of a subject with leukemia. In one embodiment, the method comprises generating a biomarker expression profile for a test sample from the subject based on a level of one or more biomarkers listed in Table 4A, and classifying, on a computer, whether the subject has a good prognosis and a low risk of relapsing leukemia or a poor prognosis and a high risk of relapsing leukemia, based on the biomarker expression profile for the test sample. Optionally, the method comprises calculating a risk score for the subject based on the biomarker expression profile. Also provided is a computer system comprising a processor configured for comparing a biomarker expression profile to one or more control profiles as described herein.

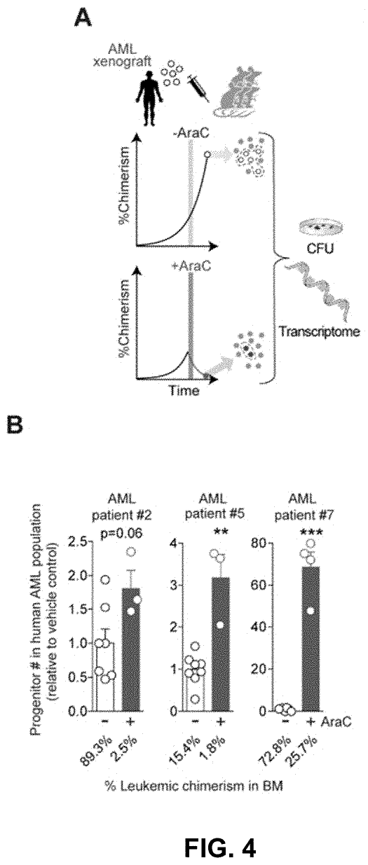

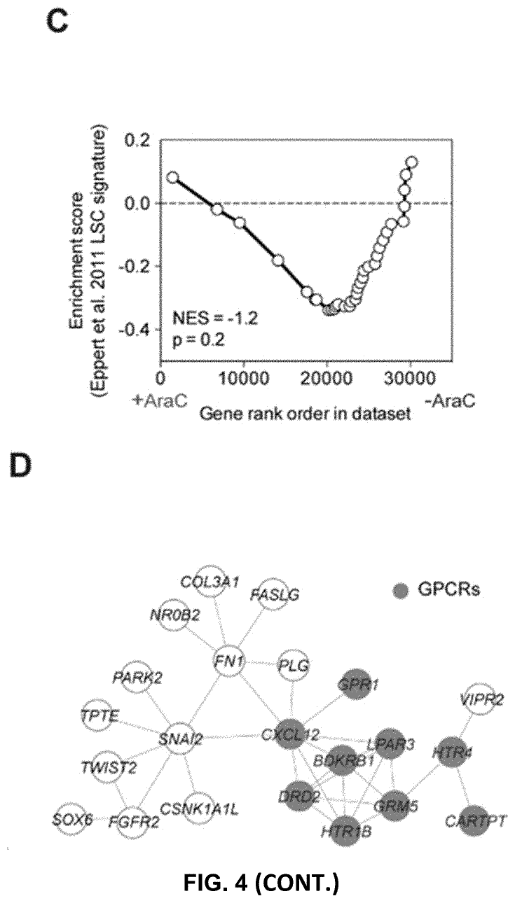

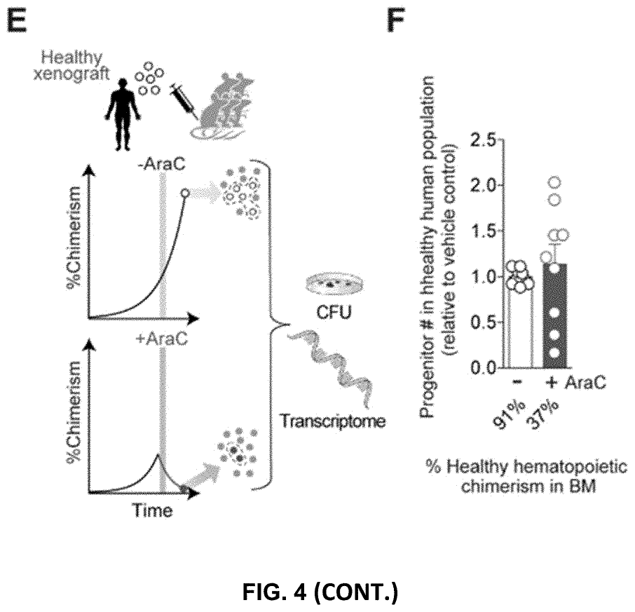

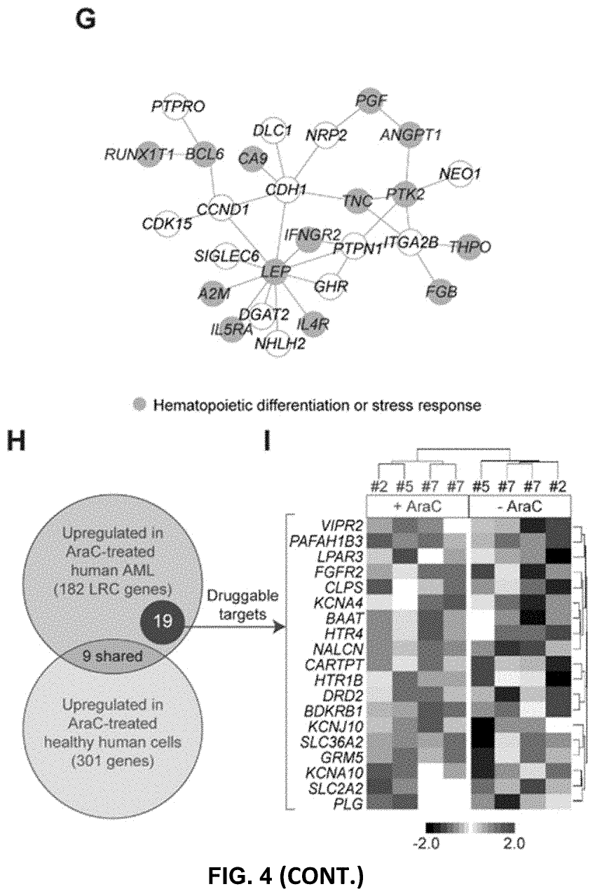

As set out in the examples and FIG. 4, chemotherapy with cytarabine effectively depletes leukemic stem cells and LRCs that emerge following the end of chemotherapy are molecularly distinct from leukemic stem cells. LRCs represent reservoirs of minimal residual disease that appear responsible for relapsing disease following cytotoxic treatments that are not present in chemotherapy naïve subjects. LRCs in subjects with leukemia and HRCs in healthy subjects were observed to emerge following cytotoxic treatment with cytarabine, 5-fluorouracil or radiation.

In one embodiment, a test sample is obtained from a subject who has completed a cytotoxic treatment for leukemia or induced injury in order to determine a prognosis for the subject and/or detect the presence or absence of LRCs. In one embodiment, the test sample is from a subject who previously received and has completed chemotherapy and/or radiation therapy. In one embodiment, chemotherapy may comprise the use or administration of a DNA synthesis inhibitor, optionally cytarabine. In one embodiment, the test sample is from a subject who previously received induction chemotherapy and/or consolidation chemotherapy. In one embodiment, chemotherapy comprises treatment with a cytotoxic agent such as cytarabine, anthracycline or 5-fluorouracil. In one embodiment, the cytotoxic treatment is sufficient to reduce the amount of leukemic and/or CD34+CD38− cells by at least 50%, 60%, 70%, 75%, 80%, 85%, 90%, 95%. 96%, 97%, 98% or 99%. In one embodiment, a cytotoxic treatment is complete when no additional administrations of a cytotoxic agent and/or radiation are planned or anticipated for the treatment of leukemia in a subject.

In one embodiment, the test sample is obtained from the subject at least 3 days, 5 days, 1 week or 10 days after completing the cytotoxic treatment for leukemia or induced injury. In one embodiment, the test sample is obtained from the subject between about 10 days and 40 days after completing the cytotoxic treatment for leukemia.

Also provided are methods for treating a subject having or suspected of having leukemia. In one embodiment, there is provided a method for inhibiting (i.e. reducing the likelihood) and/or preventing relapsing leukemia in a subject. In one embodiment, the method comprises determining a prognosis of a subject according to a method as described herein, and providing a suitable cancer treatment to the subject in need thereof according to the prognosis determined.

As shown in the Examples, targeting LRCs following chemotherapy is a particularly advantageous for inhibiting and/or preventing relapsing leukemia, optionally inhibiting and/or preventing relapsing AML. In one embodiment, there is provided a method of treating leukemia a subject in need thereof comprising administering an agent that targets Leukemic Regenerating Cells (LRCs) to the subject. In one embodiment, the subject has completed a cytotoxic treatment for leukemia. Also provided is the use of an agent that targets LRCs for treating leukemia in a subject in need thereof. In one embodiment, the agent is administered or for use at least 3 days, 5 days, 1 week or 2 weeks after completing cytotoxic therapy for leukemia.

In some embodiments, the method further comprises the co-administration or use of the agent that targets LRCs and chemotherapy, and/or the administration or use of the agent that targets LRCs prior to chemotherapy, in addition to the use of administration of the agent that targets LRCs after chemotherapy.

In one embodiment, the cytotoxic treatment comprises the administration or use of chemotherapy such as cytoreductive chemotherapy. In one embodiment, the chemotherapy comprises the administration or use of a DNA synthesis inhibitor. In one embodiment, the chemotherapy comprises the administration or use of cytarabine. As demonstrated in the Examples, cytoreductive chemotherapy with cytarabine results in a transient period of leukemic vulnerability wherein LRCs can lead to relapsing disease.

In one embodiment, the methods or uses described herein for treating a subject having or suspected of having leukemia involve the use or administration of an effective amount of an agent that targets LRCs. As used herein, the phrase “effective amount” or “therapeutically effective amount” means an amount effective, at dosages and for periods of time necessary to achieve the desired result. For example in the context or treating a leukemia such as AML, an effective amount is an amount that for example reduces the likelihood of relapsing disease compared to the response obtained without administration of the agent. Effective amounts may vary according to factors such as the disease state, age, sex and weight of the animal. The amount of a given agent that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

In one embodiment, an agent that targets LRCs is formulated for use or administration to a subject in need thereof. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999.

Various agents that target LRCs are known in the art and described herein. For example, in one embodiment, the agent that selectively targets LRCs is a DRD2 antagonist, optionally thioridazine.

As shown in the Examples and FIG. 4, a number of drug-targetable pathways capable of selectively interrupting leukemic regrowth by LRCs have been identified. In one embodiment, the agent that selectively targets LRCs is an antagonist for a gene or protein encoded by a gene selected from VIPR2, PAFAH1B3, LPAR3, FGFR2, CLPS, KCNA4, BAAT, HTR4, NALCN, CARTPT, HTR1B, DRD2, BDKRB1, KCNJ10, SLC36A2, GRM5, KCNA10, SLC2A2 and PLG. In one embodiment, the antagonist is an antisense nucleic acid molecule or compound that targets the gene through RNA interference.

For example, an antisense nucleic acid molecule may be chosen that is sufficiently complementary to the target, i.e., one that hybridizes sufficiently well and with sufficient specificity, to give the desired effect. In one embodiment, the antisense nucleic acid molecule is specifically hybridizes or is complementary to a target, such as transcript encoding for VIPR2, PAFAH1B3, LPAR3, FGFR2, CLPS, KCNA4, BAAT, HTR4, NALCN, CARTPT, HTR1B, DRD2, BDKRB1, KCNJ10, SLC36A2, GRM5, KCNA10, SLC2A2 or PLG. A skilled person will appreciate that the sequence of an antisense nucleic acid molecule need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

In one embodiment, the agent that targets LRCs is for use or administration to the subject after completing cytotoxic treatment for leukemia. For example, in one embodiment the agent is for use or administration at least 3 days, 5 days, 7 days, 10 days, 2 weeks, or at least 3 weeks after completing the cytotoxic treatment. In one embodiment, the agent is for use or administration between about 10 days and 40 days after completing the cytotoxic treatment. In one embodiment, the agent is for continuous or repeated use after completing the cytotoxic therapy for leukemia.

As set out in the Examples, targeting LRCs may prevent or reduce the likelihood of relapsing leukemia and agents that reduce the levels of LRCs in a subject after stopping cytotoxic treatment are expected to be useful candidates for the treatment of AML. Accordingly, in one embodiment there is provided a method of screening a test agent for use in preventing or inhibiting relapsing AML. In one embodiment, the method comprises:

administering the compound to a subject with AML treated with chemotherapy; obtaining a test sample from the subject following the end of chemotherapy; and

detecting a level of Leukemic Regenerating Cells (LRCs) in the test sample, wherein a compound that reduces the level of LRCs in the test sample compared to a control level is identified as a candidate compound for preventing or inhibiting relapsing AML.

In one embodiment, the subject with AML is a non-human animal, optionally a non-human transgenic animal comprising an AML xenograft. In one embodiment, detecting the level of LRCs comprises detecting one or more biomarkers listed in Table 4A.

Also provided are kits for determining a prognosis for a subject at risk of developing relapsing leukemia, the kit comprising one or more detection agents for biomarkers described herein, typically with instructions for the use thereof. In one embodiment, the kit includes detection agents such as antibodies directed against two or more biomarkers. In one embodiment, the kit includes antibodies directed against two, three or all four of SLC2A2, DRD2, FASLG and FUT3.

In one embodiment, the kit optionally includes a medium suitable for formation of an antigen-antibody complex, reagents for detection of the antigen-antibody complexes and instructions for the use thereof such as for in a method for determining a prognosis for a subject with leukemia as described herein.

The information and biomarkers described herein are useful for generating, detecting and/or isolating Leukemic Regenerating Cells (LRCs) or Hematopoietic Regenerating Cells (HRCs). In one embodiment, LRCs or HSCs are generated by exposing a subject to a cytotoxic treatment, optionally an induced injury, such as with a chemotherapeutic agent or radiation. In one embodiment, there is provided a method for detecting LRCs in a test sample comprising detecting a level of one or more biomarkers listed in Table 4A in the test sample and comparing the level of the one or more biomarkers in the test sample to one or more control levels. Also provided is a method of detecting HRCs in a test sample comprising detecting a level of one or more biomarkers listed in Table 4C in the test sample and comparing the level of the one or more biomarkers in the test sample to one or more control levels.

In one embodiment, the control levels are representative of the level of the one or more biomarkers in LRCs or HRCs and similarity between the level of the one or more biomarkers in the test sample and the one or more control levels is indicative of the presence of LRCs or HRCs respectively in the test sample. Optionally, the method further comprises isolating the LRCs and/or HSCs from the test sample in order to produce a population of isolated LRCs and/or HSCs, such as by the use of FACS.

In one embodiment, there is provided an isolated population of LRCs as described herein. In one embodiment, the LRCs express one or more of the biomarkers listed in Table 4A. In one embodiment, there is provided a cell culture comprising LRCs and a culture media.

In one embodiment, there is provided an isolated population of HRCs as described herein. In one embodiment, the HRCs express one or more of the biomarkers listed in Table 4C. Also provided is a cell culture comprising HRCs and a culture media.

In one embodiment, the culture media comprises serum from a subject previously exposed to a cytotoxic treatment, optionally cytarabine.

Optionally, the LRCs and/or HRCs described herein are isolated or selected using methods known in the art for sorting cells based on the expression of one or more biomarkers. For example, in one embodiment the step of isolating the LRCs and/or HRCs form the population of cells comprises flow cytometry, fluorescence activated cell sorting, panning, affinity column separation, or magnetic selection.

Cell cultures comprising LRCs and/or HRCs as described herein are useful in screening methods for the detection of agents for preventing or inhibiting relapsing leukemia. Accordingly, in one embodiment there is provided a method of screening a test agent for use in preventing or inhibiting relapsing leukemia, the method comprising contacting the test agent with LRCs or a cell culture containing LRCs and detecting a biological effect of the test agent on the LRCs. Different biological effects may be detected in order to screen test agents for their utility for preventing or inhibiting the relapse of leukemic disease. For example, in one embodiment, the biological effect comprises a reduction in the level of LRCs and a test agent that reduces the level of LRCs in a sample is identified as a candidate for preventing or inhibiting relapsing leukemia. Other biological effects that may be detected include changes in gene expression, such as changes in expression of one or more biomarkers listed in Table 4.

In one embodiment, agents for preventing or inhibiting relapsing leukemia selectively target LRCs relative to HRCs. Accordingly, in one embodiment the method further comprises contacting a test agent with HRCs or a cell culture comprising HRCs and detecting a biological effect of the test agent on the HRCs. In one embodiment, a test agent that exhibits a selective biological effect (such as a cytoreductive effect) for LRCs relative to HRCs is identified a candidate for preventing or inhibiting relapsing leukemia.

In one embodiment, the present disclosure provides a method for identifying and validating a test agent as a selective anti-LRC agent comprising:

contacting one or more LRCs with the test agent and one or more HRCs with the test agent;

detecting a change in one or more activities of the LRCs in response to the test agent,

detecting a change in one or more activities of the HRCs in response to the test agent; and

identifying the test agent as a selective anti-LRC agent if contact with the test agent induces one or more activities in the LRCs without inducing a comparable activity in the HRCs.

In one embodiment, the activity is apoptosis, necrosis, proliferation, cell division, differentiation, migration or movement, presence or absence of one or more biomarkers, level of one or more biomarkers, or induction thereof. In one embodiment, the biomarkers are listed in Table 4.

The above disclosure generally describes the present invention. A more complete understanding can be obtained by reference to the following specific examples of certain embodiments of the invention.

### Example 1

### Results

### Primitive AML Cells are Vulnerable to Chemotherapeutic Killing

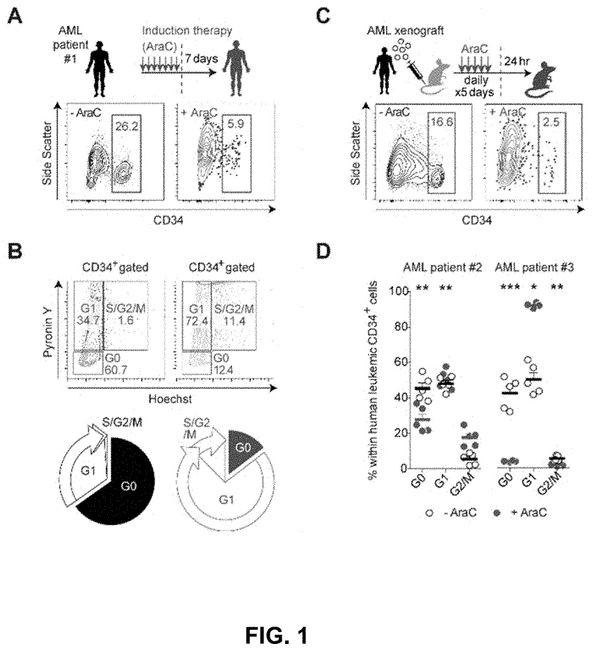

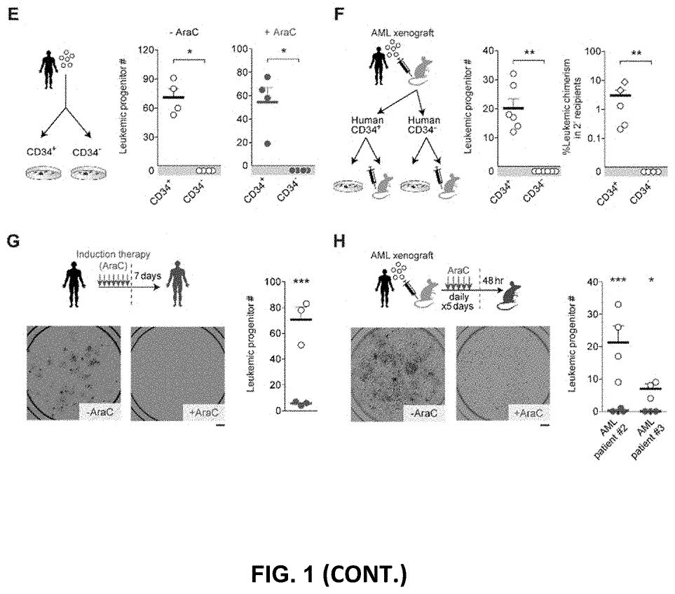

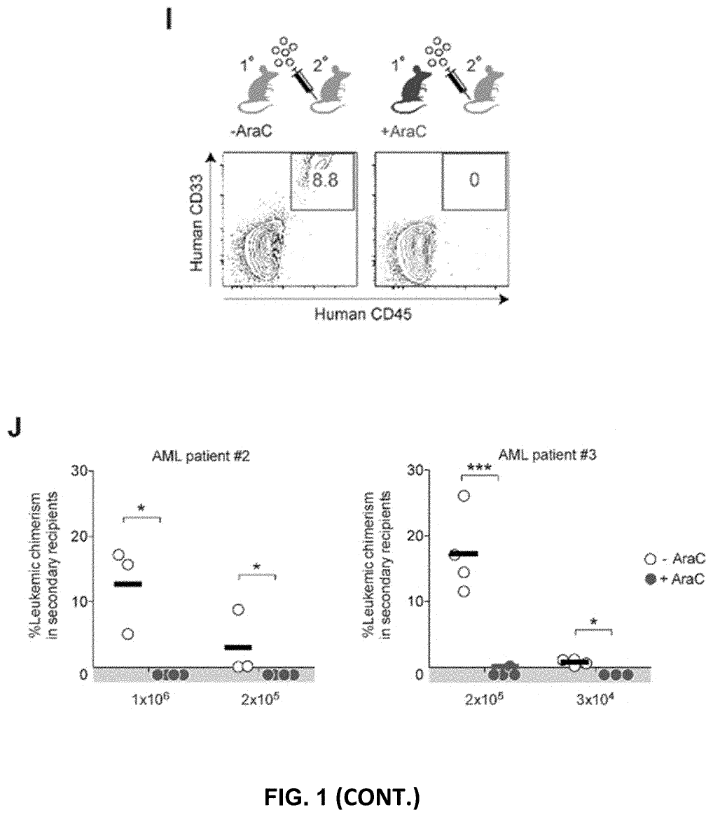

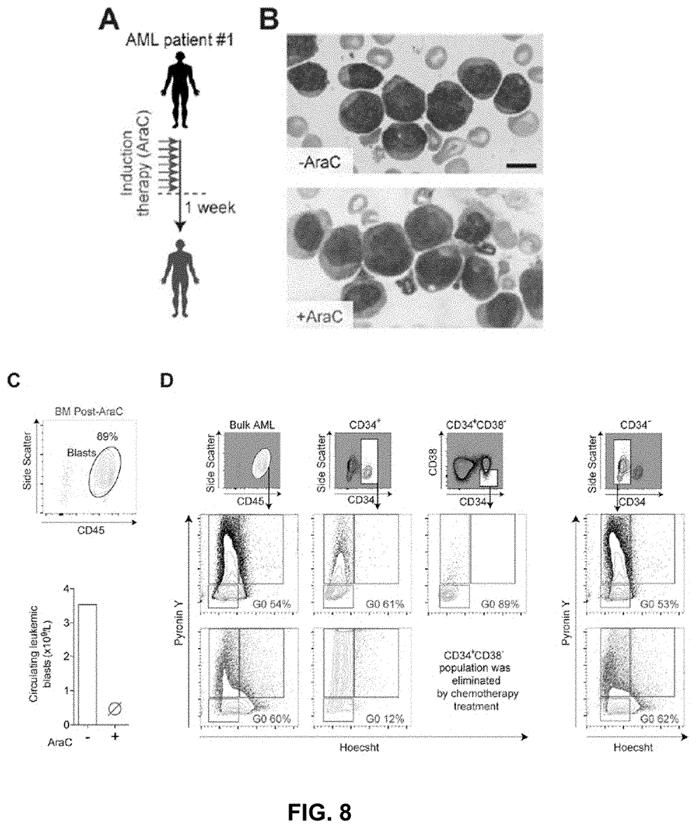

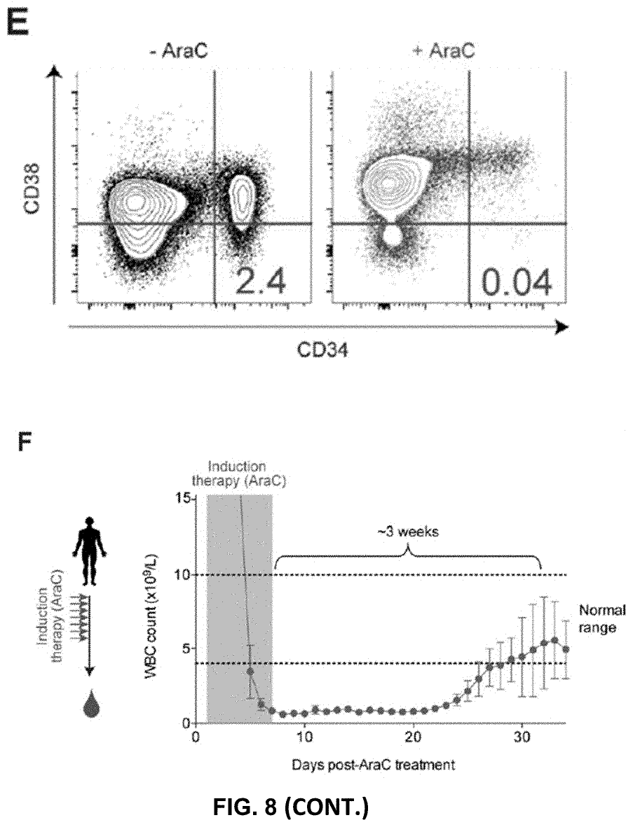

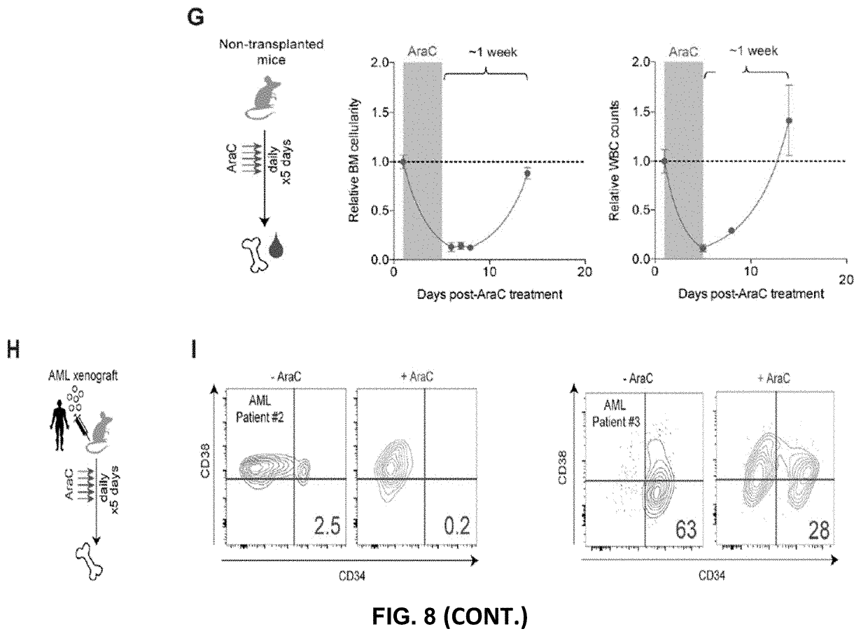

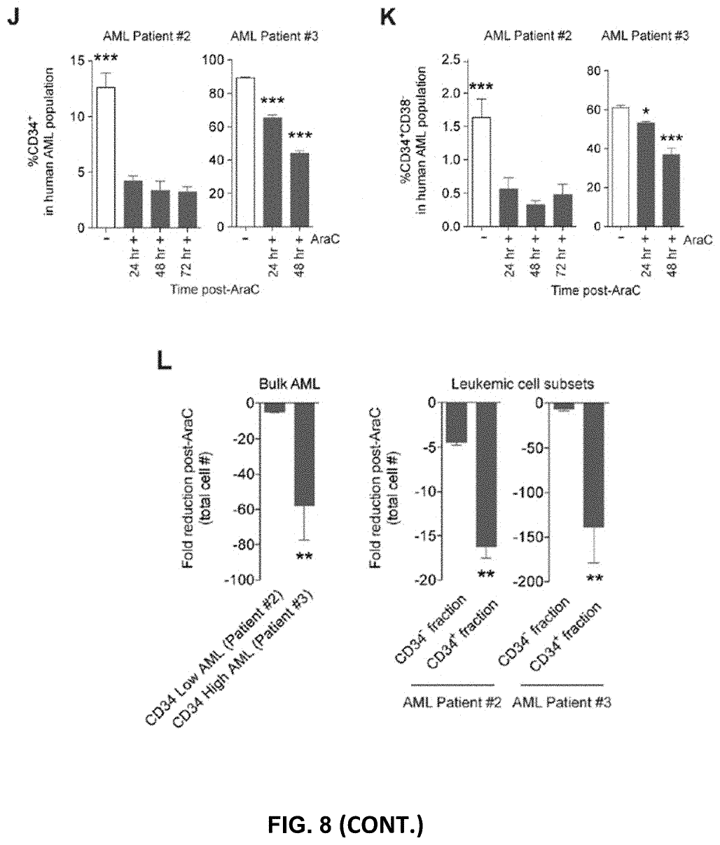

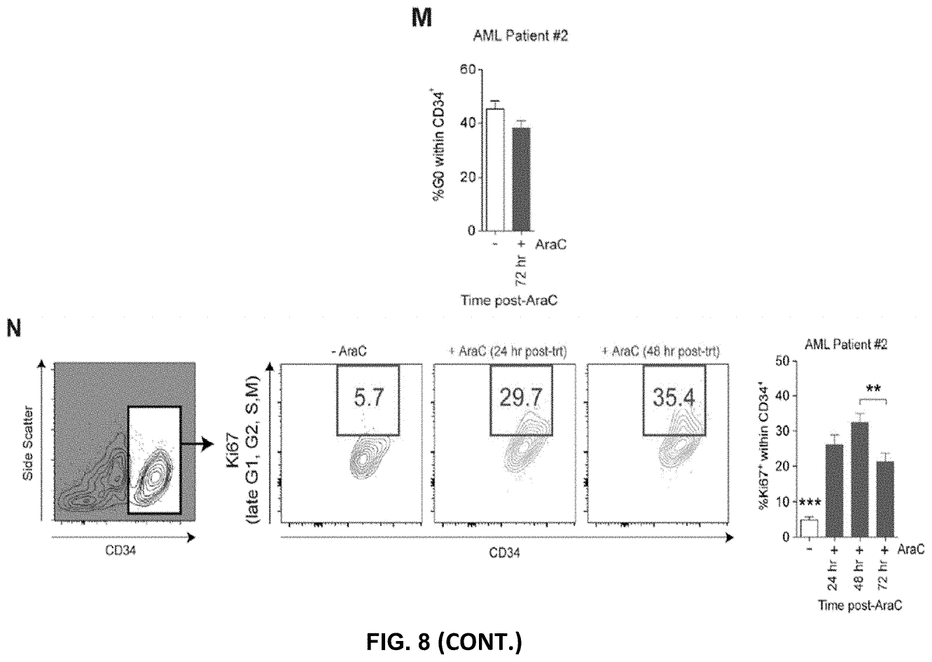

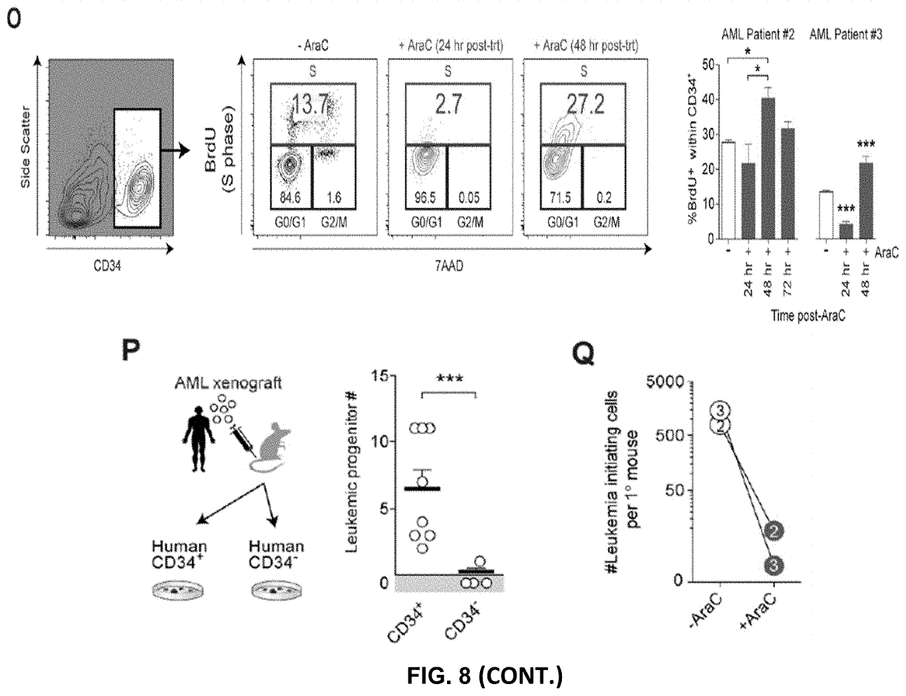

To establish a strong clinical context of AML chemotherapy response, leukemic populations that persist immediately after the completion of chemotherapy treatment were profiled. AML patient BM cells were collected prior to treatment and one week following standard induction chemotherapy as the earliest practical time for sampling post-therapy. In the absence of definitive features to discriminate healthy cells versus residual leukemic cells in remission states, a patient whose BM remained nearly entirely composed of identifiable leukemic cells was selected (FIGS. 8A-8C) despite significant cytoreductive clearance of circulating blasts (FIG. 8C). Consistent with previous reports (Ishikawa et al., 2007; Saito et al., 2010), it was observed that primitive CD34+ cells and more stringent CD34+CD38− subsets were progressively more quiescent than bulk leukemic cells before chemotherapy (FIG. 8D). However, unlike previous predictions (Ishikawa et al., 2007; Thomas and Majeti, 2017), the quiescent status of these populations did not protect them from cytotoxic insult. Despite the persistence of disease (FIG. 8A-8C), CD34+ AML cells were dramatically depleted by chemotherapy (FIG. 1A) and CD34+CD38− fractions were abolished (FIG. 8E). Post-chemotherapy, surviving CD34+ cells were no longer quiescent and had entered active cell cycle states (FIG. 1B). This suggests that chemotherapeutic insult stimulates cell cycle activity among phenotypically primitive AML cells, sensitizing them to subsequent chemotherapeutic challenge.

To experimentally corroborate this clinical observation, human AML-xenografts were used. In contrast to the difficulty of discriminating very rare leukemic cells from healthy leukocytes in patients (Levine et al., 2015), species-specific antigens allow human leukemia to be unambiguously tracked in xenograft recipients. AraC was applied as the gold standard chemotherapeutic agent included in AML treatments (Reese and Schiller, 2013) and approximated clinical schedules by extending AraC administration over the course of 5 consecutive days. Following the final AraC dose, BM cells at 24-hr intervals over a 3-day period were analyzed to 1) detail cell cycle kinetics of residual AML cells and 2) relate to patient analysis as timelines of regeneration are condensed by ˜3-fold in xenografts (FIGS. 8F and 8G). Across genetically distinct AML patient samples (Table 1), AraC treatment strongly reduced the frequencies of primitive CD34+ and CD34+CD38− cells within residual AML populations (FIGS. 1C and 8H-8K), similar to the clinical observations. In addition to the drop in CD34+ frequencies, patient grafts with high initial CD34 content revealed more robust total leukemic cytoreduction after treatment (Patient #2 vs Patient #3; FIG. 8L). This suggests that CD34+ cells do not simply acquire a different cellular identity post-therapy, but instead are physically depleted. Cell cycle assays validated the S-phase specific activity of AraC as evidenced by an initial loss of BrdU+ cells (Cannistra et al., 1989; Saito et al., 2010). Despite the selective elimination of S-phase cells, complementary Ki67 and Hoechst-Pyronin Y assays indicated that surviving CD34+ populations did not remain quiescent and had entered early stages of cell cycle progression as soon as 24 hr post-treatment (FIGS. 1D and 8N) (Bologna-Molina et al., 2013). This led to peak levels of BrdU incorporation by 48 hr, with a return to normal states by 72 hr post-AraC withdrawal (FIGS. 1D and 8M-8O). The transition from dormancy to actively cycling states by 24 hr post-treatment (FIGS. 1D and 8N) suggests that CD34+ cells were rendered susceptible to repeated treatments of AraC, given the timing of AraC administration at 24-hr intervals.

While CD34 is a valuable marker to profile leukemic stem and progenitor populations, this relationship is not universal (Thomas and Majeti, 2017). Therefore, functional assays to test CD34+ versus CD34− disease subsets for each patient examined clinically or in xenografts were applied. In all cases, self-renewal was unique to CD34+ fractions, while CD34− cells were devoid of colony-forming progenitors or leukemia-initiating capacity (FIGS. 1E, 1F, and 8P). Furthermore, this potential remained exclusive to the CD34+ fraction of patient leukemic cells after chemotherapy treatment (FIG. 1E), indicating that regenerative activity remains connected to the CD34+ cell identity over the course of chemotherapy exposure. Therefore, the loss of CD34+ leukemic cells indicates a biologically meaningful change in disease properties in response to chemotherapy. As predicted by cellular phenotypes (FIG. 1A), functional profiling of bulk leukemic cells revealed a reduction of progenitor activity in AML patient BM cells as a result of chemotherapy treatment (FIG. 1G) despite disease persistence (FIGS. 8A-8C). Parallel experiments were performed using human AML cells purified from xenograft BM 48 hr after AraC withdrawal, timed to characterize the cellular composition as immediately as possible while ensuring clearance of intracellular AraC and its metabolites (Liliemark et al., 1987). As seen clinically, functional progenitors were depleted from residual leukemic populations that survived AraC treatment in xenografts (FIG. 1H). Serial LIC transplantation assays showed that AraC also suppressed functional LSCs (FIGS. 1I-1J and Table 2), mirroring the in vitro results. Accounting for the overall decrease in AML disease cellularity, this translated to an overwhelming loss of LSCs per AraC-treated recipient (FIG. 8Q), consistent with previous reports (Farge et al., 2017; Griessinger et al., 2014). Alternative to suggested expectations that LSCs are preferentially spared by cytoreductive chemotherapy (Jordan et al., 2006), the patient and xenograft data build on previous findings and indicate that primitive AML cells become recruited into the cell cycle over the course of multi-dose chemotherapy treatment, leading to their quantitative depletion.

### Chemotherapy Uniquely Induces Aggressive Leukemic Re-Growth Versus Healthy Hematopoiesis

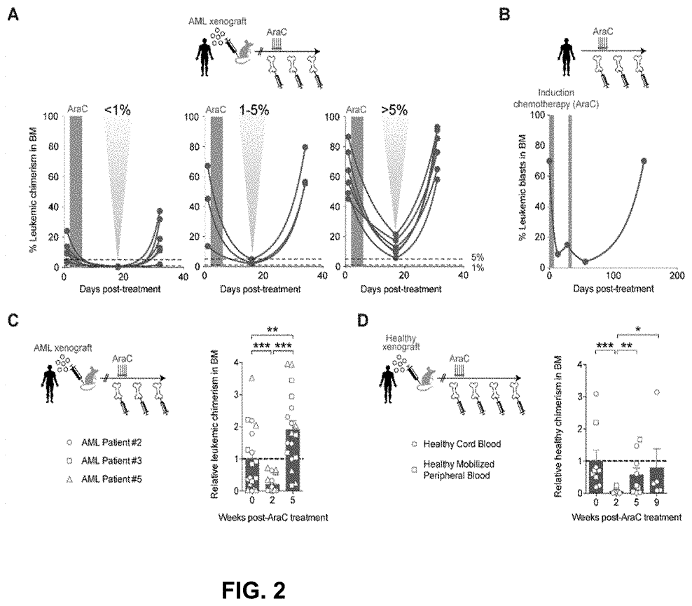

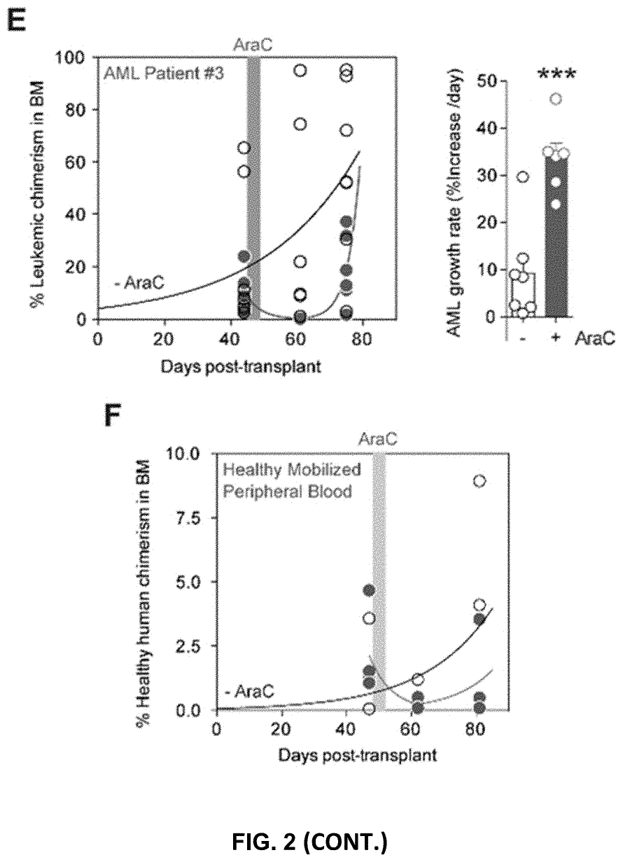

To understand how leukemia regenerates despite AraC substantially reducing functional LSC pools, whether relapsed AML would re-develop over time if primary recipient mice were maintained was examined. As LIC assays are transplantation-dependent, they likely introduce technical variables (Sun et al., 2014) that do not apply to AML regeneration in patients. Therefore, serial BM aspirate sampling allowed us to mimic clinical standards of response assessment (FIG. 2A). Despite initial disease suppression in response to chemotherapy, all mice experienced abrupt regeneration of AML disease with time (FIG. 2A). This pattern was shared across xenografts from genetically distinct AML patients and was conserved whether or not disease burden was reduced below typical clinical remission thresholds of <5% (FIG. 2A). These leukemic re-growth dynamics closely mirror clinical chemotherapy responses in AML patients, where disease recurrence regularly develops following a short-lived phase of blast reduction (FIG. 2B).

To determine whether these re-growth patterns were unique to AML, separate groups of mice with healthy hematopoietic stem cells (HSCs) were reconstituted, and in vivo AraC treatment of both sets of transplanted mice in parallel (FIGS. 2C and 2D) were performed. AML regrowth consistently surpassed pre-treatment levels of disease across 3 patient samples (FIG. 2C). However, healthy HSC-initiated grafts ultimately respected the boundary of their original BM reconstitution levels established before AraC treatment (FIG. 2D). These conservative patterns of healthy regeneration were not limited to adult sources of human HSCs, but were also seen upon AraC challenge of cord blood-derived HSCs (FIG. 2D), which are highly regenerative (Ueda et al., 2001). Extended follow-up of cord blood grafts reflected restrained patterns of growth even at 9 weeks post-AraC treatment (FIG. 2D), nearly twice the duration of the AML graft monitoring.

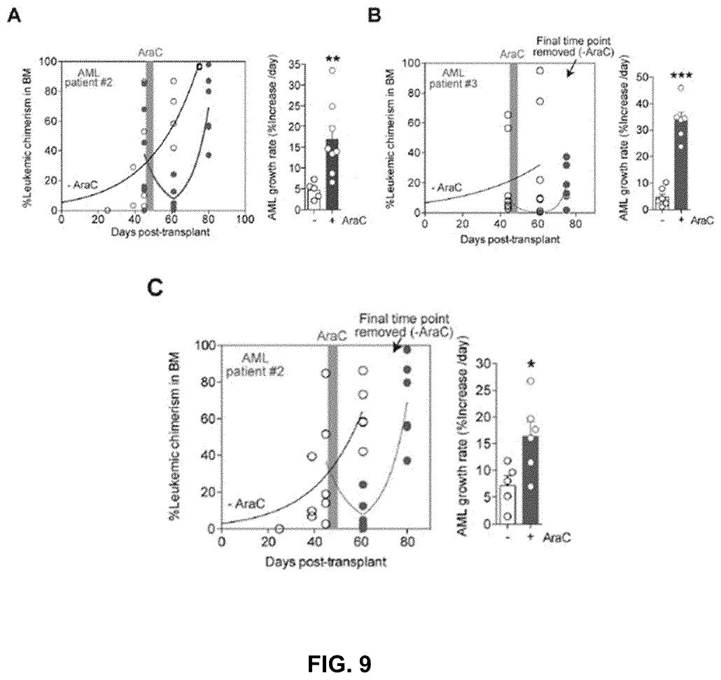

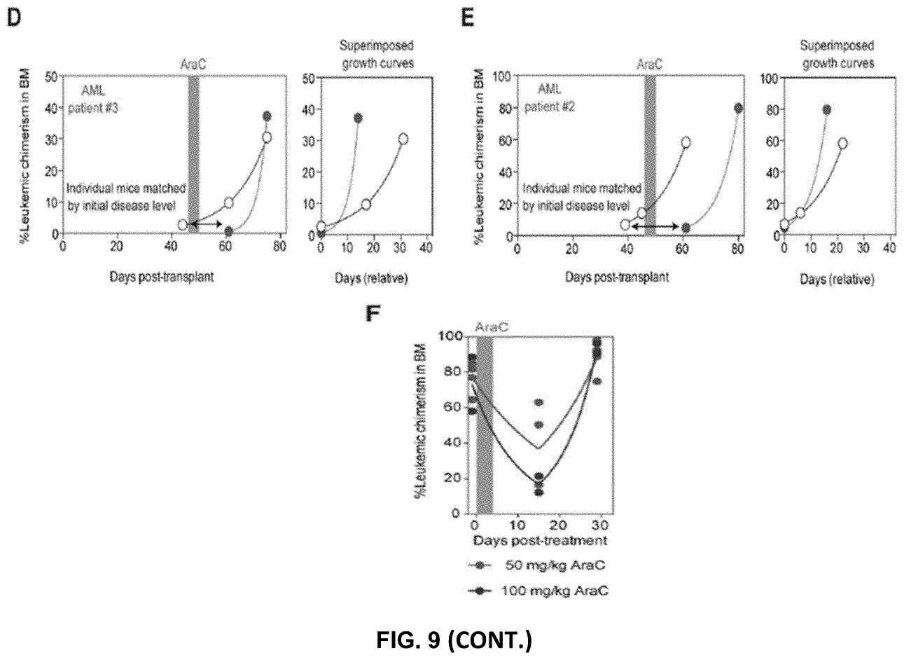

Xenograft modeling uniquely allows multiple experimental conditions to be tested for a single patient's disease. Accordingly, internal controls not exposed to AraC allowed us to evaluate the causal influences of AraC on AML re-growth behavior. Quantitative kinetic modeling indicated that AraC treatment provoked accelerated leukemic growth in comparison to vehicle-treated controls (FIGS. 2E and 9A). This difference was not dependent on the extent of disease saturation within BM, as leukemic growth rates differed between vehicle- and AraC-treated mice at comparable levels of leukemic burden (FIGS. 9B-9E). In contrast to AML, healthy human hematopoiesis showed disciplined patterns of re-growth after AraC cytoreduction, with rates matching those of vehicle-treated controls (FIG. 2F). These results suggest disparate biological properties of regeneration between AML disease and healthy human hematopoiesis in response to chemotherapy.

Next, whether AraC dose intensification from 50 mg/kg to 100 mg/kg would impact leukemic re-growth was explored. This more aggressive regime produced unacceptable treatment-related mortality rates of 60%, which was not balanced by any therapeutic benefit in the few mice that survived. Although the higher AraC dose initially achieved more profound cytoreduction of human AML, disease recurrence occurred simultaneously in the two dose conditions (FIG. 9F). These limitations of standard AraC therapy highlight the need to better characterize the origins of AML relapse to guide the development of more durably effective therapies.

### Cellular Characterization of In Vivo Leukemic Regeneration Post-Chemotherapy

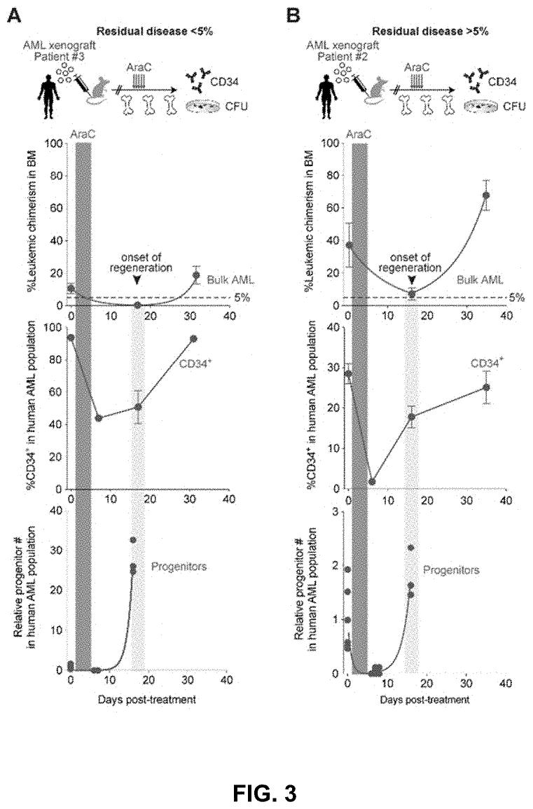

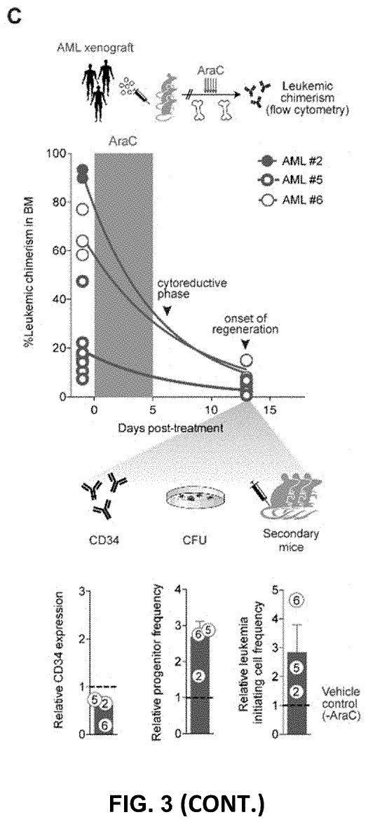

To investigate the cellular dynamics that shape relapse development, kinetic profiling was applied as a guide to select landmark events during leukemic re-growth (FIGS. 3A and 3B) which allowed us to identify a time point that represented a transition from downward trajectories of leukemic disease post-therapy towards the onset of bulk disease regeneration (FIGS. 3A and 3B). At this transitional stage, percentages of CD34+ cells had begun to recover from initial suppression but had not yet returned to pre-treatment states. Across patient samples, CD34+ content was only fully restored once relapsed disease was grossly evident (FIGS. 3A and 3B). Beyond phenotype assessments, residual leukemic cells at this stage of disease re-growth for functional interrogation were also purified. Despite the incomplete replenishment of CD34 expression, colony-forming progenitors had rebounded, surpassing their initial frequencies prior to AraC (FIGS. 3A and 3B). This suggests that both CD34 phenotypes and functional regenerative potential share an upward trend of recovery; however, at this state of regeneration, CD34 expression alone does not fully predict the increased functional activity relative to therapy-naive disease (FIGS. 3A and 3B). This chronology was conserved whether or not the disease burden descended below traditional thresholds of remission (i.e., <5% BM cells; FIGS. 3A vs. 3B) (Estey and Dohner, 2006), and was reproducible across independent patient genotypes.

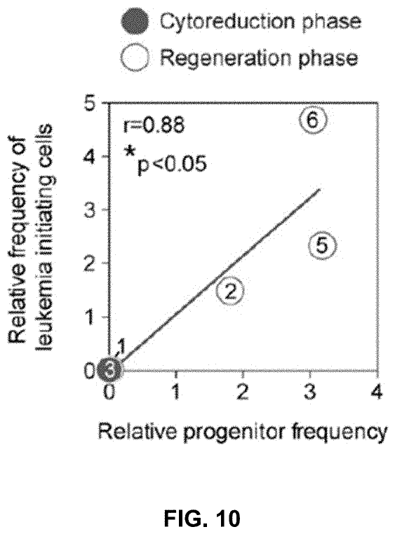

Given the functional significance of the regenerative turning point, the diversity of patient samples examined at this stage post-therapy was expanded and the analysis was broadened to include LIC assays by limiting dilution serial transplantation (FIG. 3C and Table 3). This reinforced that the reestablishment of CD34+ pools is delayed relative to the surge of functional activity at the onset of overt disease regeneration (FIG. 3C). Furthermore, functional in vitro and in vivo measures of self-renewal were closely correlated (FIG. 10), indicating that in vitro CFU assays are reliable surrogates to detect leukemic regeneration. Taken together, kinetic analyses indicate that reassembly of AML disease is sequential in nature, where regenerating AML cells with self-renewal potential emerge as a founder population to drive re-growth of bulk leukemic disease in response to chemotherapy treatment.

Leukemic Regenerating Cells are Molecularly Distinct from Therapy-Naive LSCs

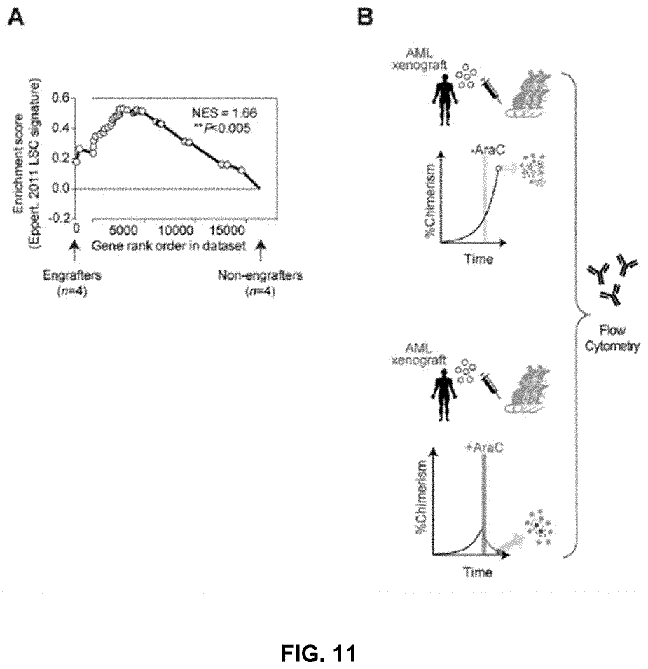

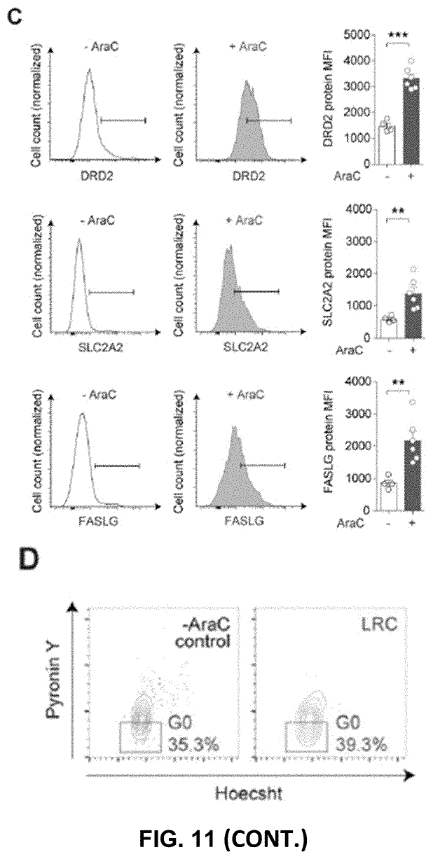

The next aim was to molecularly characterize the leukemic state that represents the origins of disease re-emergence. Accordingly, human AML cells from xenograft BM were purified for parallel functional and molecular analysis, by comparing leukemic cells recovered at the onset of AraC-driven regeneration to vehicle-treated controls (FIG. 4A). Across genetically distinct patient xenografts (Table 1), CFU assays confirmed the highly clonogenic capacity of leukemic cells at the brink of re-growth post-AraC (FIG. 4B). Despite this functional validation, regenerative AML cells were devoid of gene expression signatures used to characterize LSCs in the absence of chemotherapy i.e., therapy-naive LSCs (FIG. 4C) (Eppert et al., 2011). Importantly, traditional LSC gene expression signatures correlated with LIC activity of AML patient samples before treatment initiation (FIG. 11A), unlike xenografts post-AraC. This suggests that chemotherapy treatment disconnects regenerative activity from molecular profiles typical of therapy-naive LSCs.

Unbiased analyses further revealed the unique transcriptional features of human AML at the onset of regeneration post-AraC, termed Leukemic Regenerating Cells (LRCs). A total of 191 protein-coding genes were selectively upregulated after AraC exposure relative to matched vehicle-treated controls (Table 4), and these changes were validated at the protein level (FIGS. 11B and 11C). Gene lists associated with cell proliferation were not prominent among LRC molecular signatures (Table 4), reflecting flow cytometry evidence that cell cycle profiles had re-normalized by this point (FIG. 11D). Instead, STRING network analysis identified functional associations that were highly enriched for G-protein coupled receptor signaling (FIG. 4D), which was a salient theme of the LRC gene signature (Table 4) and offers targeting potential.

To determine the specificity of this molecular profile to AML LRCs, the same experimental approach using xenografts reconstituted with healthy human hematopoietic cells (FIG. 4E) were reproduced. Along equivalent timelines that had revealed an expansion of AML progenitors post-AraC treatment, healthy progenitor frequencies were restored but remained within normal ranges (FIG. 4F vs. FIG. 4B), consistent with the disciplined kinetics of regeneration exhibited by normal hematopoietic grafts (FIGS. 2D and 2F). Gene expression profiles paralleled these functional properties of healthy hematopoietic regeneration (Table 4). Specifically, closely networked genes expressed by healthy regenerating cells related to stress responses and hematopoietic differentiation, representing appropriate biological processes related to healthy hematopoietic recovery (FIG. 4G and Table 4). Importantly, the profiling of healthy AraC-exposed cells identified multiple genes previously linked to hematopoietic regeneration in response to acute myelotoxic stress caused by chemotherapy or radiation (e.g. ANGPT1 and HMOX1; Table 4) (Cao et al., 2008; Zhou et al., 2015). Both of these genes have been reported to moderate the regenerative process towards normalization and re-establishment of homeostatic growth dynamics following acute injury (Cao et al., 2008; Zhou et al., 2015), consistent with the restrained regenerative growth that was observed (FIGS. 2D and 2F). The absence of these growth-limiting signals in AML-LRC gene expression profiles (Table 4) reinforces the uncontrolled nature of leukemic regeneration compared to healthy hematopoiesis.

The next aim was to identify drug-targetable pathways capable of selectively interrupting leukemic re-growth while sparing healthy hematopoietic recovery following AraC treatment. Comparative analysis allowed us to refine the leukemic regeneration profiles by excluding genes shared with healthy regenerating cells (FIG. 4H). To prioritize LRC-specific features with therapeutic value, a filtering step to capture candidates with known antagonists based on the Drug-Gene Interaction database (FIGS. 4H and 4I) was applied. This identified a focused set of 19 genes including DRD2 and HTR1B, which are both monoamine GPCRs linked to AML self-renewal properties (Sachlos et al., 2012)(Etxabe et al., 2017). None of these targets overlap with LSC signatures reported to date (Eppert et al., 2011; Ng et al., 2016), suggesting that actively regenerating leukemia acquires features that could not be predicted from therapy-naive disease states.

### Cell-Extrinsic Factors Mediate Regenerative Features of AML Post-Chemotherapy

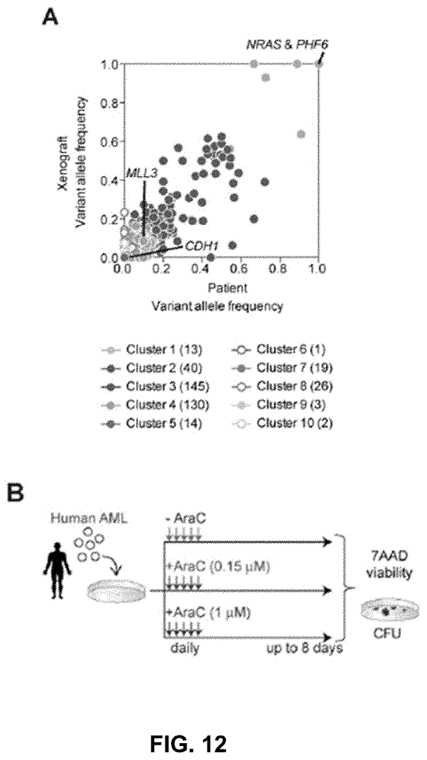

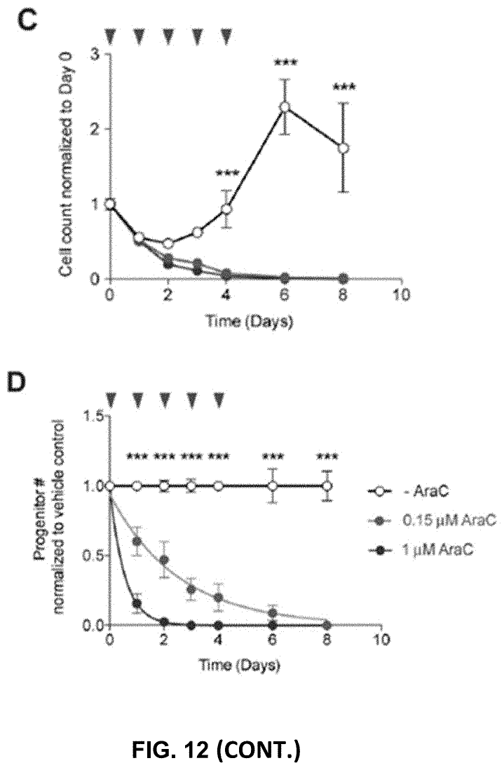

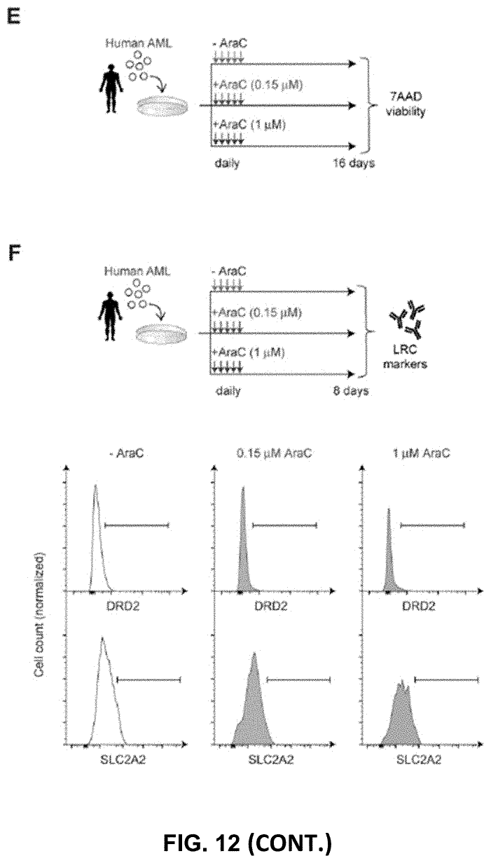

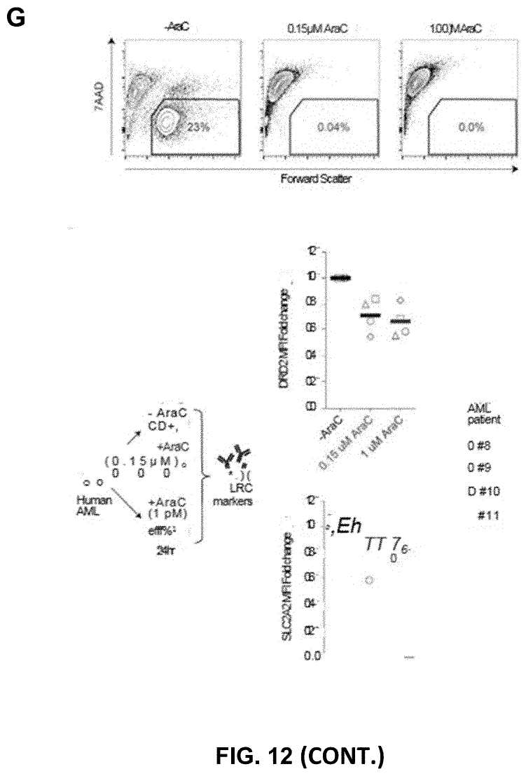

To determine whether LRC gene signatures develop primarily from permanent genetic changes or whether they represent a reversible plastic state, whole genome sequencing before and after AraC challenge in the xenograft model was performed. In a genetically complex AML sample, the complement of genetic subclones was preserved from the de novo patient cells through AraC therapy and regenerative disease re-growth in xenografts (FIG. 12A), suggesting that all genetic lineages of the disease persisted and contributed to the regeneration process. AraC treatment did not introduce additional chromosomal instability or mutations among genes thought to have a causative role in AML pathogenesis (Papaemmanuil et al., 2016) (Table 5). This does not preclude a connection between AraC treatment and genomic evolution in AML, as shorter relapse durations have been associated with less extensive genetic progression in longitudinal studies of clinically treated AML patients (Hirsch et al., 2016; Kronke et al., 2013) and the rapid kinetics of relapse in xenografts may account for the lack of genomic evolution in the model. Regardless, the observation indicates that LRC phenotypes and aggressive re-growth characteristics can arise even in the absence of major genetic changes, suggesting factors other than genomic mutations could participate in leukemic regeneration.

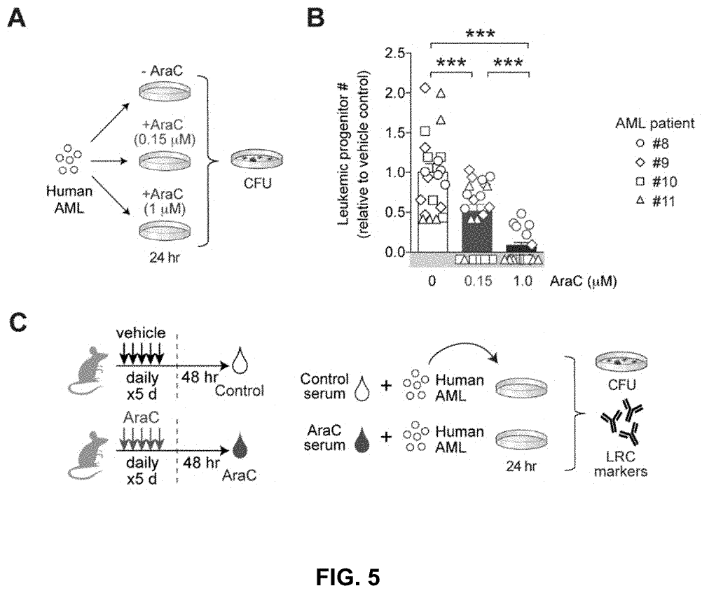

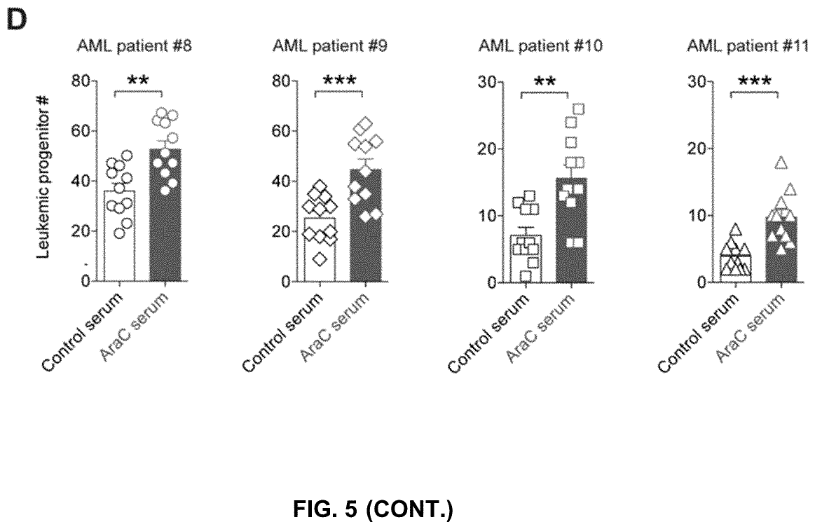

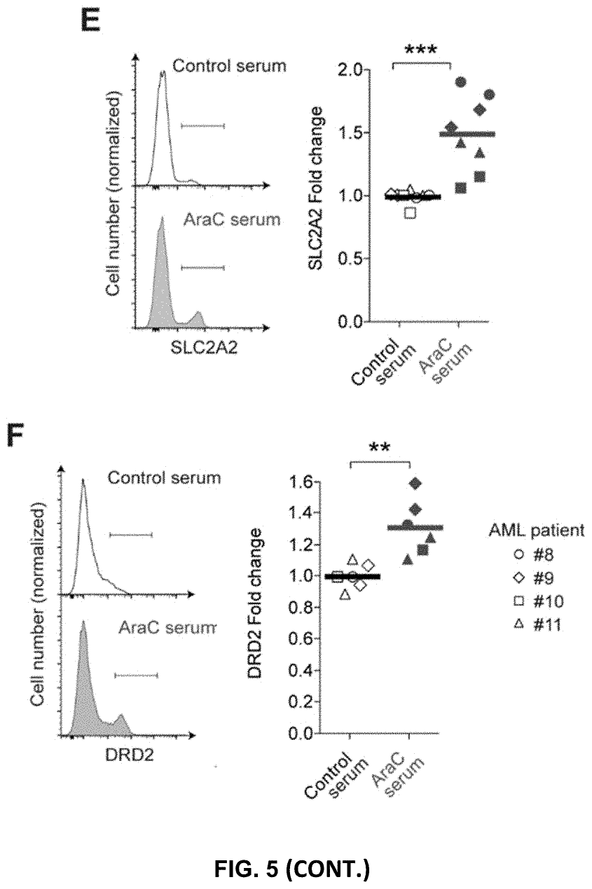

To explore the basis of LRC regulation, in vitro platforms to test whether human AML cells can activate LRC features as a cell-intrinsic response to AraC exposure were applied. The addition of AraC to human leukemic cell cultures consistently depleted functional progenitors in both serum-supplemented (FIGS. 5A and 5B) and serum-free conditions (FIGS. 12B-12D). In longitudinal time series, no evidence of progenitor recovery was observed, even after eliminating AraC from the culture (FIGS. 12B-12D). Extended culture for two weeks post-AraC led to a complete loss of viable leukemic cells despite continued survival of control cultures (FIG. 12E), highlighting the lack of functional regeneration response in vitro, unlike the dynamics observed following in vivo AraC treatment (FIGS. 2 and 3). These in vitro residual leukemic cells also lacked expression of LRC-specific markers (FIGS. 12F and 12G), collectively suggesting that an in vivo setting is required to support LRC emergence following AraC treatment.

To reconcile the in vivo versus in vitro observations, whether signals released into the circulation following in vivo AraC injury could be sufficient to stimulate LRC activity from therapy-naive AML cells was tested. Accordingly, serum was collected from immune-deficient mice recovering from AraC or vehicle-treated controls and added to human AML cell cultures (FIG. 5C). Impressively, heightened progenitor activity was detected among leukemic cells cultured with serum from AraC-exposed mice, consistently across 4 AML patient samples (FIG. 5D). This regenerative behavior was accompanied by enriched expression of LRC markers (FIGS. 5E and 5F), reinforcing the connection between functional and molecular hallmarks of leukemic regeneration. Serum-borne AraC could not have mediated these effects as it is rapidly eliminated from circulation in vivo (Zuber et al., 2009), and direct culture with AraC had neutral or opposite effects on the same samples under equivalent conditions (FIGS. 5B and 12G). The inability to induce LRC features via in vitro AraC exposure suggests that the in vivo environment is required to promote regenerative states of leukemic disease.

### AML LRCs can be Uniquely Targeted to Interrupt Disease Recurrence In Vivo

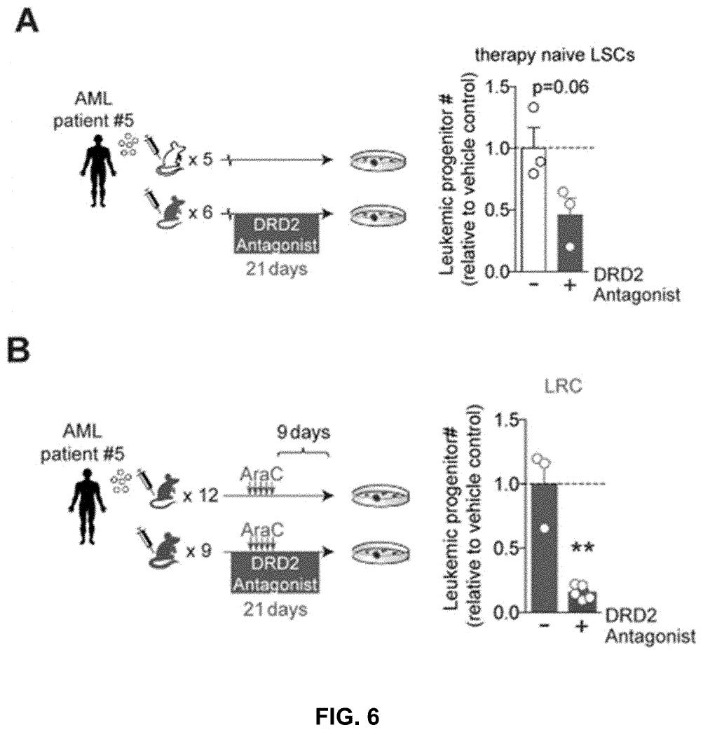

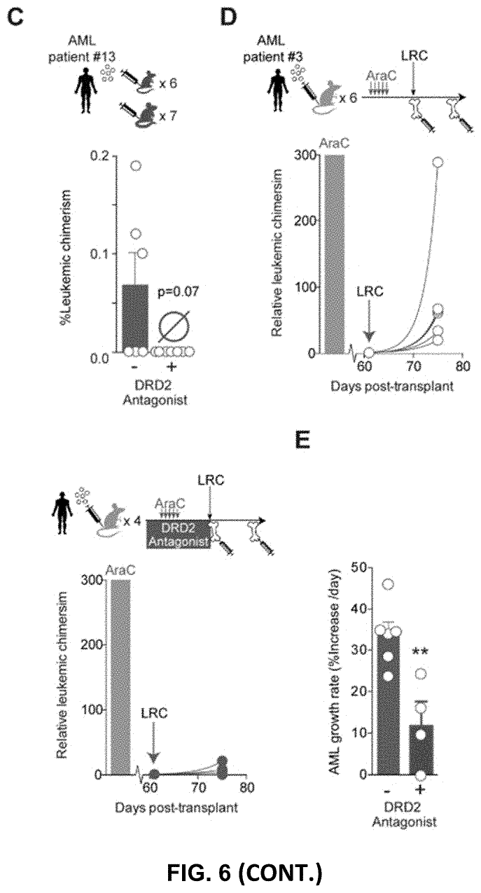

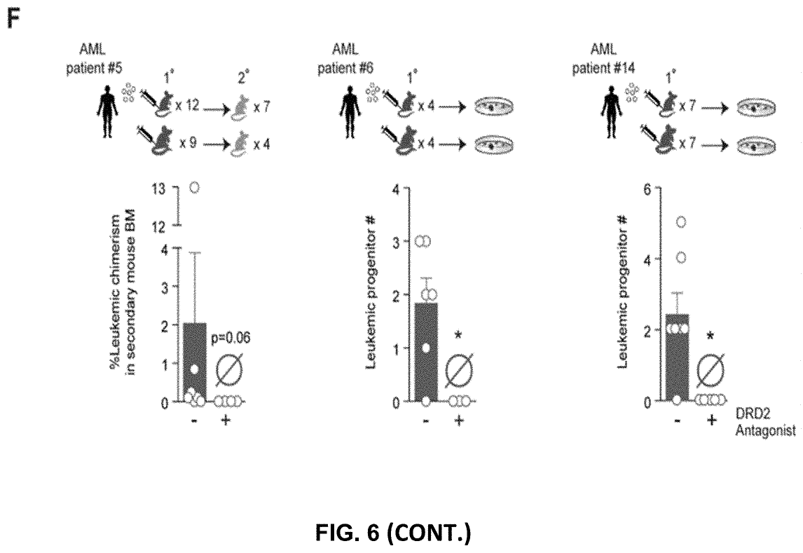

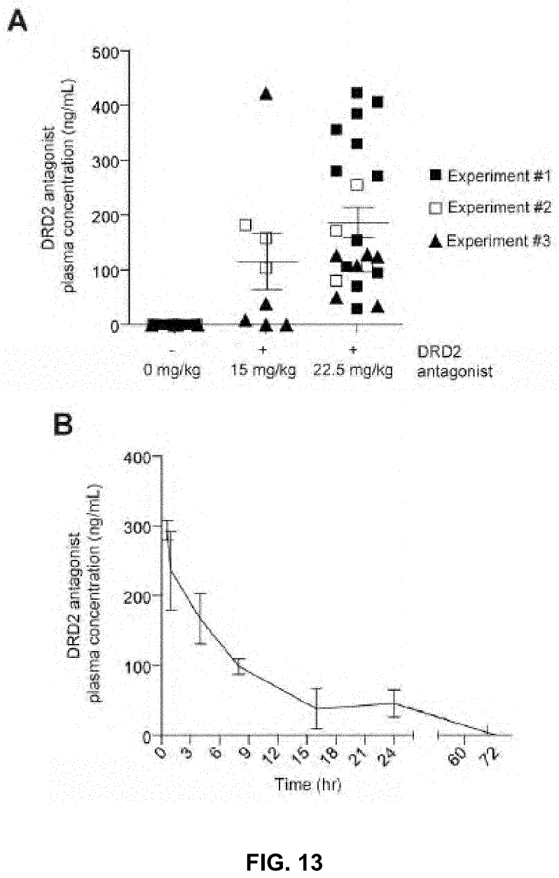

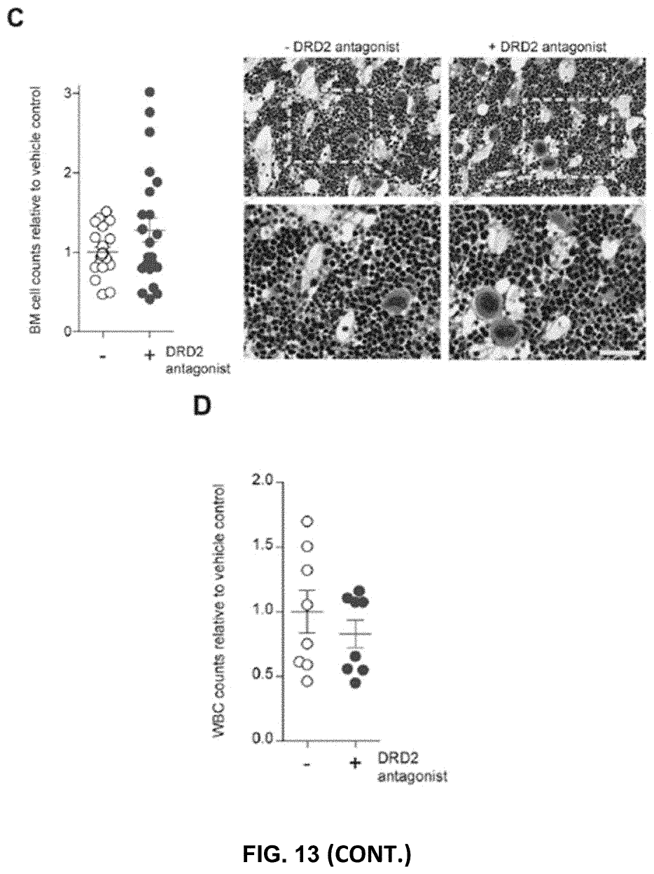

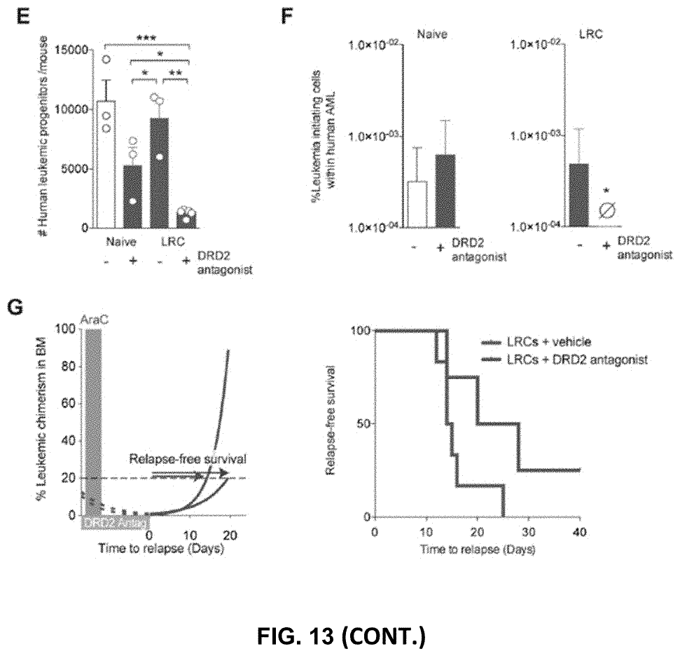

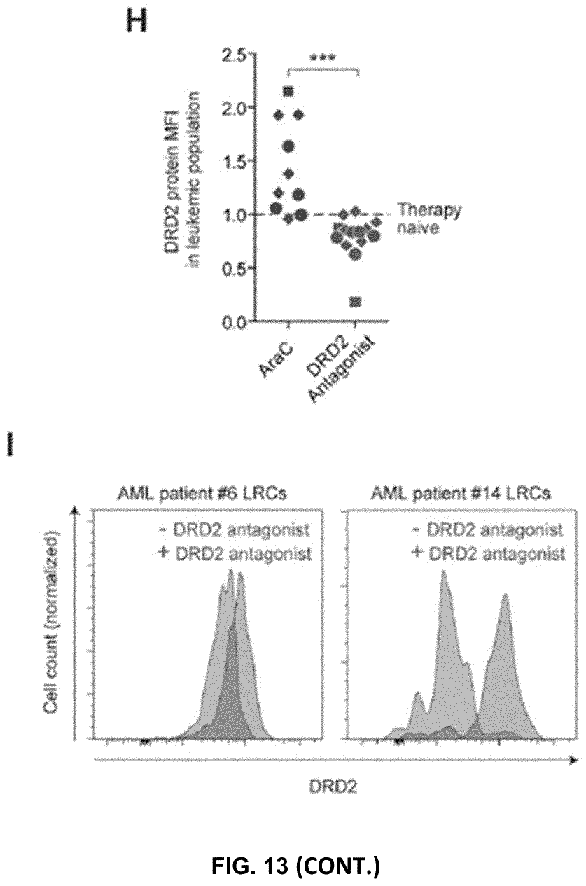

As LRC development following AraC treatment is exclusively an in vivo phenomenon, it was rationalized that any LRC-targeted intervention approach would require in vivo evaluation. Given the molecular differences that distinguish LRCs from traditionally characterized LSCs (FIG. 4C), preclinical xenograft experiments were structured to evaluate the functional differences of targeting LRCs versus therapy-naive LSCs (FIGS. 6A and 6B). As DRD2 is one of 19 druggable candidates preferentially expressed by LRCs (FIG. 4I), a small molecule antagonist of DRD2 that has been shown to suppress LIC activity in ex vivo AML cultures (Sachlos et al., 2012) was used. First, in vivo DRD2 antagonist administration to AML xenograft recipients (FIGS. 13A-13D) was optimized. Then, the effects of DRD2 antagonist therapy in AML-xenografts populated with therapy-naive LSCs versus xenografts that harbored LRCs as a result of AraC exposure were compared. DRD2 antagonist treatment began 5 days prior to AraC introduction to ensure stabilized steady-state levels throughout the period of chemotherapy exposure, and anti-DRD2 therapy was maintained until the characterized point of LRC emergence 9 days following AraC withdrawal. The day following the final DRD2 antagonist treatment, human leukemic cells were purified from xenograft BM to evaluate progenitor activity in vitro and by serial transplantation (FIGS. 6A, 6B, S6E, and S6F). In vivo DRD2 antagonism moderately affected AML progenitors arising from therapy-naive LSCs (FIG. 6A) but had profound effects on regenerating LRCs (FIGS. 6B and 13E). DRD2 antagonist treatment did not impact the LIC content of therapy-naïve AML (FIG. 13F) as measured by serial transplantation. However, DRD2 antagonism strongly affected AraC-exposed LRCs as demonstrated by a complete loss of LIC activity (FIG. 13F), consistent with the overexpression of DRD2 in the LRC state (FIG. 4I). These results demonstrate the distinct biological properties of LSCs versus LRCs beyond transcriptional signatures alone.

Given the potential benefit of DRD2 antagonism against LRCs, its therapeutic efficacy relative to AraC chemotherapy alone was evaluated. Xenografts derived from AML patient #13 demonstrated the most dramatic therapeutic response to AraC. However, even in this favorable scenario, residual disease persisted in 50% of recipient mice (FIG. 6C). The addition of DRD2 antagonist treatment achieved disease-free status in 100% of recipients (FIG. 6C). Using a more aggressive case of AML (Patient #3), BM sampling confirmed measurable residual leukemic disease across all recipient mice following AraC intervention (FIG. 6D), allowing the kinetics of disease regeneration to be comparatively evaluated over time. In contrast to the abrupt trajectories of leukemic re-growth after AraC treatment alone, leukemic growth rates were successfully disrupted by the incorporation of DRD2 antagonist (FIGS. 6D and 6E), which nearly doubled the time to overt relapse (FIG. 13G). While absolute time scales cannot be translated directly from xenografts to human timelines, two-fold prolongation of progression-free survival is considered highly promising in human oncology trials (Finn et al., 2016).

Based on this initial evidence, the analysis was expanded to include a wider spectrum of AML patient genotypes (FIG. 6F). While in vivo delivery of DRD2 antagonist did not improve the overall cytoreductive activity of AraC (Table 6), LRC targeting reproducibly blocked disease regeneration potential across all three additional patient samples tested (FIG. 6F). This loss of disease re-initiation capacity was measured by secondary LIC assays and progenitor assays in vitro (FIG. 6F) and coincided with loss of DRD2 protein expression (FIGS. 13H and 13I). Collectively the findings demonstrate that leukemic regeneration can be inhibited by targeting the unique LRC state that emerges as a result of cytoreductive chemotherapy treatment.

### Features of LRCs Emerge in Human AML Patients and Predict Relapse

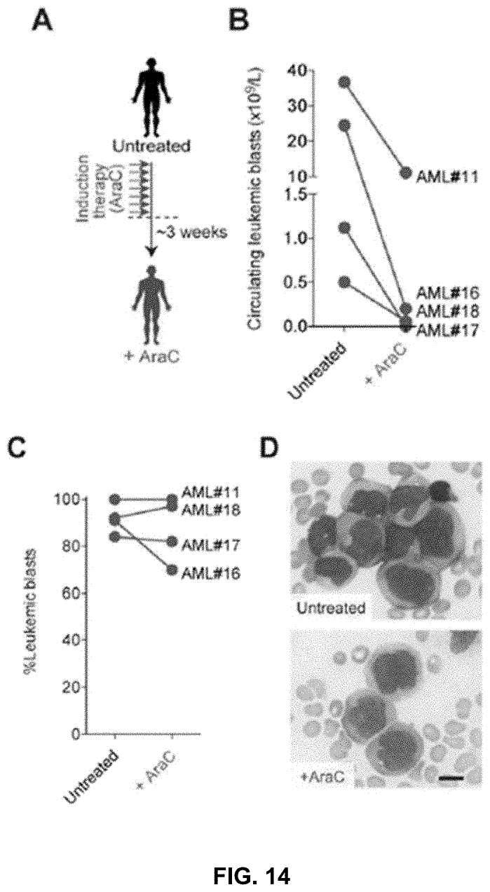

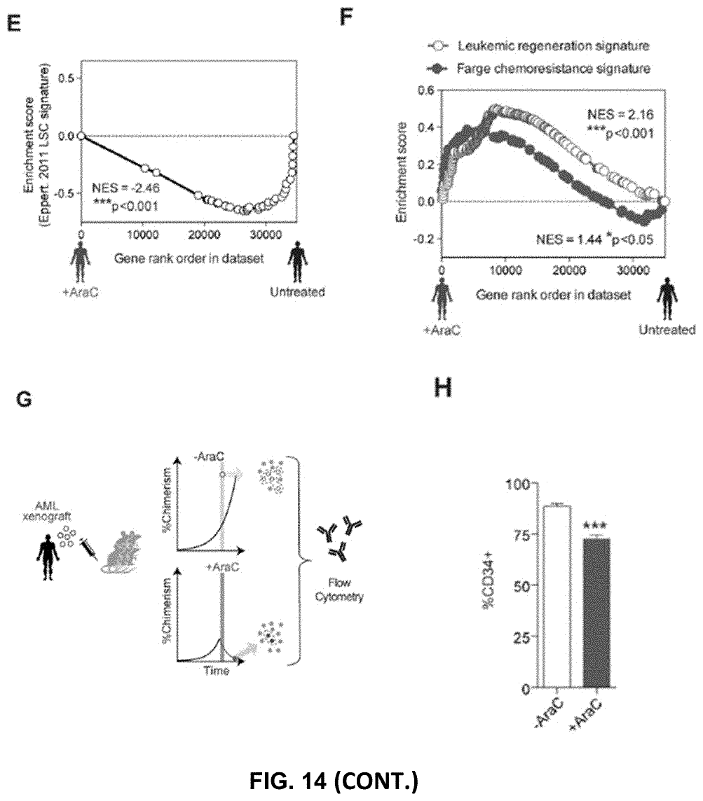

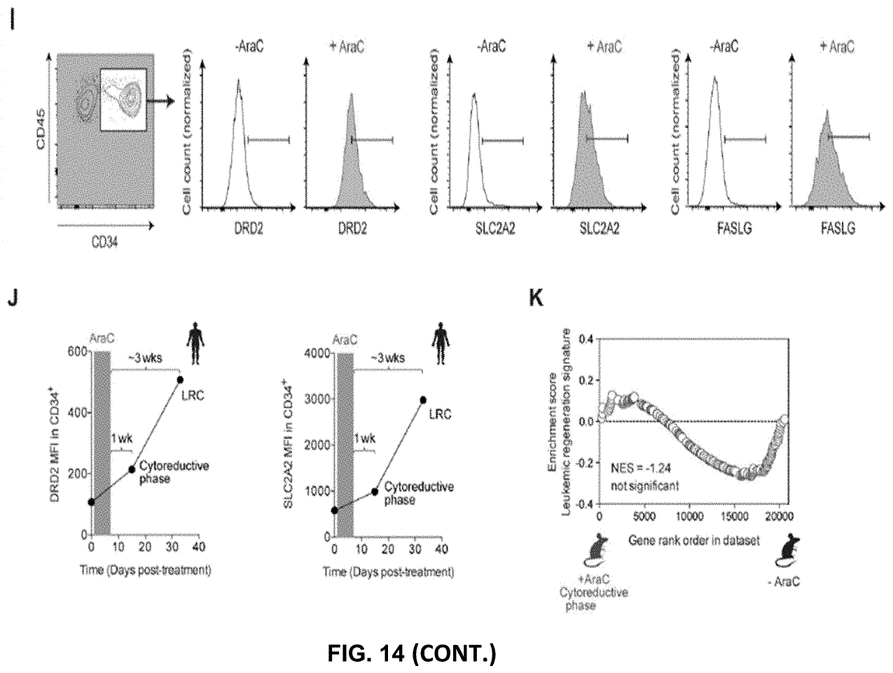

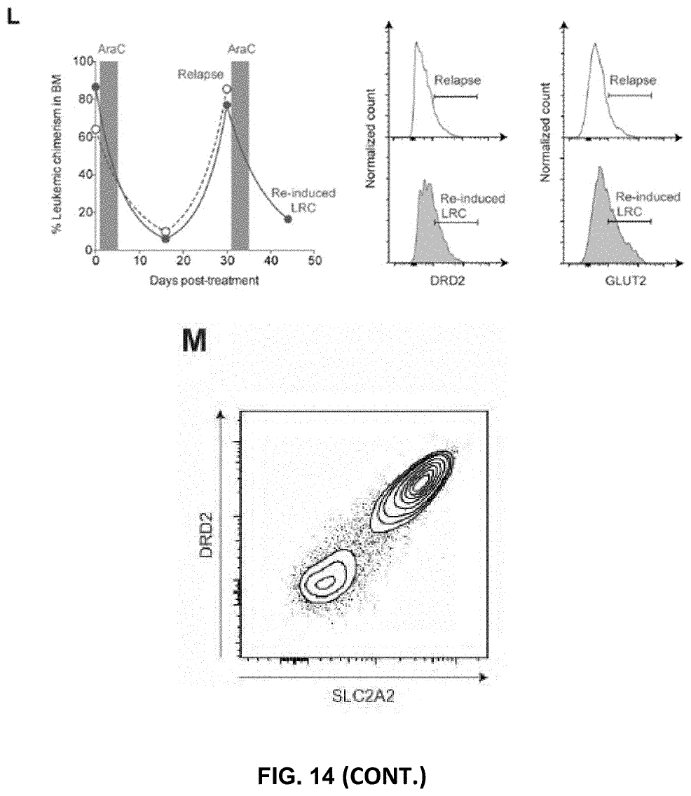

To evaluate the clinical relevance of LRCs, how closely the findings in xenografts translate to disease regeneration in human patients was examined. To correspond with timelines of regeneration profiled in preclinical models, BM aspirates were obtained from consenting patients approximately 3 weeks following the completion of standard induction chemotherapy (FIGS. 8F and 8G). To ensure suitable purity of leukemic cells for analysis, samples from patients whose BM disease persisted post-treatment despite successful blast reduction in peripheral circulation (FIGS. 14A-14D) was prioritized. Using these AML cells from human BM, in vitro progenitor assays were performed in tandem with global transcriptome analysis (FIG. 7A). In contrast to evidence that chemotherapy initially depletes leukemic progenitors (FIG. 1G), progenitor activity became enriched among residual leukemic cells by this later time point of assessment (FIG. 7B). Despite the peak of self-renewal activity seen at this later point of chemotherapy response, the same patient-derived cells lacked gene expression signatures related to therapy-naive LSCs (FIGS. 7C and 14E) (Eppert et al., 2011; Ng et al., 2016). Instead, these highly regenerative AML cells preferentially expressed the LRC signature FIG. 7C). Expression profiles of general chemoresistance and cytoreductive stress were also detectable at this stage (Farge et al., 2017), although to a lesser extent than LRC signatures (FIG. 14F).

Flow cytometry was used to further characterize residual leukemic populations at the single cell level post-chemotherapy. In response to chemotherapy, CD34+ frequencies dropped among remaining leukemic blast populations (FIG. 7D), reinforcing the conclusion that tracking CD34 alone may not be sufficient to detect and monitor residual disease. In contrast, protein markers of LRCs were reliably upregulated within the same disease subsets post-therapy (FIG. 7E). When LRC markers were profiled with CD34, co-expressing cells were abundant during regenerative periods post-chemotherapy treatment, whereas LRC protein expression had been negligible or absent among CD34+ leukemic cells prior to therapy (FIG. 7F). Similar patterns of co-expression were reproduced in experimental AML xenografts (FIG. 14G-14I). These findings suggest that in response to therapy, phenotypically primitive subsets acquire new properties during regenerative states, as opposed to quantitative expansion of the CD34+ population itself. Temporal profiling of AML patient BM showed that these changes develop gradually over the course of chemotherapy response and are not an immediate consequence of cytoreduction (FIG. 14J). LRC gene signatures do not develop immediately after chemotherapy exposure in xenografts either (FIG. 14K). Beyond the conserved sequence of events that shape chemotherapy response, an amalgamated transcriptional analysis demonstrated the consistency of LRC gene expression patterns across both human patients and AML xenografts (FIG. 7G).

Next, the LRC signature was applied to AML patient disease evolution from initial diagnosis to initial chemotherapy response and relapse. The LRC signature was exclusively observed as part of the active chemotherapeutic response and was not found at diagnosis or upon re-establishment of AML disease at relapse (FIG. 7H). Xenograft experiments further revealed the ability to re-induce LRC marker expression by additional AraC treatment of relapsed disease (FIG. 14L). Overall, these data indicate that LRC molecular profiles arise temporarily following cytoreductive chemotherapy treatment, providing a window of therapeutic opportunity to target the LRC molecular state prior to relapse onset.

Finally, the significance of the LRC signature to minimal residual disease in human AML patients was examined. BM samples were obtained from seven patients in clinical remission following standard induction chemotherapy. Four patients relapsed within 6-13 months, and the remaining three patients remained in disease-free remission for at least 5 years (FIG. 7I). To exclude maturing lineages of healthy hematopoietic cells, the authors focused within CD34+ subsets. SLC2A2 was chosen as a representative LRC marker and was confirmed to have overlapping expression with DRD2 (FIG. 14M). Remarkably, chemotherapy treatment increased LRC marker expression exclusively in cases where a residual burden of disease remained (i.e., primary refractory disease or eventual relapse; FIG. 7J). The absence of this pattern in patients with long-term healthy recovery highlights the specificity of LRC markers for diseased versus healthy states of regeneration. Consistently, SLC2A2 expression at remission stratified patient cases to discriminate sustained remission versus eventual relapse (FIG. 7K). The two patients with the highest levels of SLC2A2 were examined in more detail. SLC2A2+ versus SLC2A2− subfractions were purified, and genomic DNA was assessed. Genetic probes for patient-specific NPM1 mutations revealed that diseased cells were preferentially enriched within the SLC2A2+ compartment (FIG. 7L). These results suggest that LRC populations represent reservoirs of residual disease, and LRC marker expression levels can be linked to clinical outcomes of AML relapse.

### DISCUSSION

The current study comprehensively profiles the in vivo cellular and molecular dynamics of human AML disease before, during, and after chemotherapy treatment. The data here along with the initial results of Farge et al (2017) now reveal that LSCs are not selectively resistant to chemotherapy. By extending the study of chemotherapy response beyond initial cytoreductive periods post-treatment, the onset of AML regeneration that leads to relapsed disease was uniquely identified. This revealed a molecular profile of LRCs that is conserved across genetically diverse cases of human AML but absent in healthy hematopoietic regenerating stem cells. Based on this distinction, the application of LRC markers permits discrimination between impending relapse versus durable disease-free survival in human AML patients during remission states. Proof-of-principle experiments using pre-clinical xenograft models further demonstrated that LRC-targeting therapy effectively restrains features of leukemic regrowth post-chemotherapy. These targets of leukemic regeneration could not have been predicted by existing characterizations of leukemic disease, as cellular states of AML during this vulnerable regenerative period are distinct from therapy-naive LSCs (Eppert et al., 2011), early stages of cytoreduction post-therapy (Farge et al., 2017) or terminal phases of relapse (Ding et al., 2012; Hackl et al., 2015; Ho et al., 2016; Shlush et al., 2017) that have previously been studied.

The findings contribute to an important emerging view that LSCs are not as resistant to chemotherapy as currently believed. It is proposed that like healthy stem cell populations that become activated in response to injury (Wilson et al., 2008), reservoirs of primitive AML cells also transition out of dormancy to replenish the supply of leukemic blasts. Because this occurs as a rapid cellular response, this can compromise the ability of primitive AML cells to resist chemotherapy when applied at repeated doses across brief time intervals. These findings complement the premise of “timed sequential therapy”, where chemotherapy delivery is strategically synchronized to match proliferative states of disease that develop in response to previous chemotherapy treatment (Burke et al., 1977). These concepts were extended from bulk AML disease to rare LSC populations and it was proposed that through these mechanisms, conventional chemotherapy protocols accomplish more effective LSC elimination than is currently recognized. As a result, it is suggested that therapeutic efforts should be re-directed towards preventing the powerful regenerative response that ensues, when functional pools of leukemic cells rebuild prior to overt recurrence of disease.

Following initial cytoreduction, the delayed appearance of LRC-specific signatures suggests that this is an adaptively acquired state of AML cells in response to in vivo AraC treatment, rather than chemotherapeutic selection of a minor pre-existing population. However, it is possible that the cells that manifest this state may not be transient themselves. For example, LRCs may include a subset of LSCs that have temporarily acquired distinct molecular features as part of the AraC treatment response. The findings suggest that an in vivo setting is required to induce states of leukemic regeneration, as in vitro AraC treatment fails to recapitulate functional or molecular hallmarks of LRCs while disease-regenerating potential can be rescued by signals released in vivo post-AraC treatment. These observations complement recent insights that the BM microenvironment contributes meaningfully to the dynamics of therapy response in human leukemia (Ebinger et al., 2016; Passaro et al., 2017) and mirror historical findings where leukemic cell proliferation could be stimulated in vitro by exposure to serum from patients who were recovering from chemotherapy treatment (Burke et al., 1977). This regenerative behavior was then related to a unique molecular state that can be therapeutically exploited to inhibit disease relapse. Given the dynamic nature of LRC properties, it will be important to further examine the optimal development and application of LRC-targeted therapies, including the refinement of treatment timing and duration.

The preclinical experiments indicate that DRD2 signaling represents a promising axis for LRC-directed targeting, providing a strong rationale to investigate other LRC-related pathways identified by the molecular characterization. Future studies should also prospectively explore LRC markers as potential prognostic/disease monitoring tools, as they could have value to improve detection sensitivity for minimal residual disease. Ultimately, the authors hope the findings highlight the importance of evaluating dynamic responses to existing chemotherapeutic drugs, which will ultimately assist in applying this paradigm to identify analogous periods of vulnerability after chemotherapy treatment of other cancers/solid tumors (Huang 2014; Kurtova et al., 2015). Accordingly, the state of CSCs in response to chemotherapy must be evaluated carefully to tailor the most effective treatment strategies (Pollyea et al., 2014), and these approaches must consider the kinetics of disease regeneration responses where the biology of cancer cells may be vastly different from steady-state disease.

### EXPERIMENTAL PROCEDURES

### Experimental Model and Subject Details

### Primary Human Hematopoietic Samples

Healthy human hematopoietic cells were isolated from BM and mobilized peripheral blood of adult donors or from umbilical cord blood. Primary AML specimens were obtained from peripheral blood apheresis or BM aspirates of consenting AML patients. AML patients examined over the course of chemotherapy treatment received standard induction chemotherapy regimens consisting of 7-day infusions of cytarabine (100 mg/m2) plus daunorubicin on days 1-3 (60 mg/m2). AML samples and adult sources of healthy hematopoietic tissue were provided by Juravinski Hospital and Cancer Centre and London Health Sciences Centre (University of Western Ontario). The Labour and Delivery Clinic at the McMaster Children's Hospital provided healthy cord blood samples. All samples were obtained from informed consenting donors in accordance with approved protocols by the Research Ethics Board at McMaster University and the London Health Sciences Centre, University of Western Ontario. Details of AML patient samples are outlined in Table 1. Mononuclear cells (MNCs) were recovered by density gradient centrifugation (Ficoll-Paque Premium; GE Healthcare) followed by red blood cell lysis using ammonium chloride solution (Stemcell Technologies). Lineage depletion of healthy hematopoietic samples was carried out using EasySep immunomagnetic cell separation (Stemcell Technologies), according to the manufacturer's instructions.

### Murine Recipients and Xenograft Assays

Mice were bred and maintained at the McMaster Stem Cell and Cancer Research Institute animal barrier facility. All experimental procedures were approved by the Animal Council of McMaster University. NOD/SCID or NSG mice were used as xenograft recipients, and xenotransplantation was performed as previously described (Boyd et al., 2014). Briefly, 6-10 week old recipient mice were sublethally irradiated (200-350 Rads, using a 137Cs γ-irradiator) 24 hours prior to intravenous transplantation of primary human samples. Both male and female mice were used, however sex was controlled within individual experiments. 6-12 weeks following transplantation, BM cells were recovered by mechanical dissociation and analyzed by flow cytometry. BM cellularity was quantified using trypan blue exclusion.

To evaluate functional LSC content, human AML cells were serially transplanted into secondary recipients by intravenous injection. BM cells were pooled from multiple primary recipients of the same group and injected into secondary mice at multiple cell doses. The threshold for engraftment detection was set at ≥0.1% human chimerism. Functional LSC frequencies were estimated using ELDA software (WEHI Bioinformatics). To evaluate functional progenitor content, xenografted human cells were purified by fluorescence activated cell sorting (FACS) or by mouse cell exclusion using magnetic cell isolation (mouse CD45 and mouse Ter119; Miltenyi Biotec) and subsequently seeded in methylcellulose.

Longitudinal in vivo monitoring of human leukemic chimerism was carried out by serial BM aspiration. 5-10 μl of BM cells were collected from femurs of anesthetized recipient mice; the procedure repeated at bi-monthly intervals on alternate femurs. Cellular growth rates were calculated as derived from the rate constant “k” of the fitted exponential growth model.

For in vivo therapy testing, mice were treated with either AraC (Sigma-Aldrich), DRD2 antagonist thioridazine (Sigma-Aldrich), or both in combination, once human grafts were established (3 weeks post-transplant). AraC was delivered daily by subcutaneous injections over five consecutive days at doses optimized by both ourselves and similar to others (Farge et al., 2017). Unless specified otherwise, AraC was delivered at 50 mg/kg, prepared in saline. DRD2 antagonist treatment was delivered by daily intraperitoneal injections over 21 consecutive days (22.5 mg/kg, prepared in 30% captisol from Ligand Pharmaceuticals). In combination regimens, AraC was introduced on Day 7 of DRD2 antagonist treatment. Weekly weight measurements were used to ensure that an appropriate dose per weight ratio was sustained throughout each treatment. Mice were allocated to drug treatment groups based on pre-treatment BM aspirates, to ensure similar starting levels of human chimerism across groups. If no initial assessment of chimerism was performed, mice were randomly allocated to experimental groups, assuring that cage mates were distributed across different groups. In experiments where residual human AML cells were isolated for functional testing post-treatment, cells were allocated to serial transplantation and/or methylcellulose progenitor assays based on the total number cells recovered as well as the known requirements for cell number input for the respective assays (characterized independently for different AML patient samples).

Whole blood was collected from the superficial temporal vein of non-transplanted NOD SCID mice recovering from AraC cytoreduction (48 hours following the completion of 5 daily doses at 50 mg/kg) or from saline-injected vehicle controls. Blood was allowed to clot for 45 minutes at room temperature and then was centrifuged at 4° C. at 2000×g for 15 minutes. Serum supernatant was collected and centrifuged for another 5 minutes to remove any residual hematopoietic cells.

### Method Details

### Liquid Cell Culture

Primary AML samples were cultured in IMDM (Gibco) containing 20% serum obtained from mice recovering from AraC cytoreduction to test whether soluble circulating factors contribute to LRC responses. As a control, the same primary AML samples were cultured in IMDM (Gibco) containing 20% serum obtained from vehicle-treated mice. Control cultures were treated with either 0.1% DMSO control or AraC (0.15 and 1.0 μM). After 24 hours of culture, the cells were collected for flow cytometry and progenitor assays. Cultures for each AML patient sample were performed with at least 3 biologically independent serum samples per condition.

Additional experimental controls to test the effect of chemotherapy in vitro included alternate culture conditions optimized for long-term maintenance of the stem/progenitor hematopoietic cells. This included StemSpan medium (Stemcell Technologies), supplemented with 100 ng/mL stem cell factor, 100 ng/mL Fms-related tyrosine kinase 3 ligand, and 20 ng/mL thrombopoietin (all sourced from R&D systems). Cells were incubated with 0.15 μM AraC, 1.0 μM AraC, or 0.1% DMSO (vehicle control). Half-media changes were performed daily to refresh AraC or vehicle control, for a period of 5 consecutive days. Following the 5-day treatment period, a full media change was performed. Beyond this point, half-media changes were performed every other day. At 1-2 day intervals throughout the culture period, cells were collected for viability assessments, flow cytometry and progenitor assays. Across conditions, equal numbers of viable cells were plated into methylcellulose for progenitor assays.

### Methylcellulose Progenitor Assays

Primary AML samples were cultured in IMDM (Gibco) containing 20% serum obtained from mice recovering from AraC cytoreduction to test whether soluble circulating factors contribute to LRC responses. As a control, the same primary AML samples were cultured in IMDM (Gibco) containing 20% serum obtained from vehicle-treated mice. Control cultures were treated with either 0.1% DMSO control or AraC (0.15 and 1.0 μM). After 24 hours of culture, the cells were collected for flow cytometry and progenitor assays. Cultures for each AML patient sample were performed with at least 3 biologically independent serum samples per condition.

### Fluorescence-Activated Cell Sorting (FACS) and Flow Cytometry

Immunophenotyping for human hematopoietic cell surface markers was carried out using the following antibodies: V450-conjugated anti-CD45 (1:100; 2D1), APC-conjugated anti-CD33 (1:300; WM-33), PE-conjugated anti-CD34 (1:200; 581), FITC- or PE-conjugated anti-CD38 (1:100 or 1:500; HB7), FITC-conjugated anti-CD19 (1:100; HIB19), and APC-conjugated anti-CD3 (1:100; UCHT1; all from BD). In order to evaluate candidates from the LRC gene signature at the protein level, gene targets were identified with available commercial antibodies that had been validated for flow cytometry. Directly conjugated antibodies were used to detect human SLC2A2 (Alexa fluor 488-conjugated; 1:100; 199017; Novus Biologicals) and FASLG (FITC-conjugated; 1:100; SB93A; Thermo Fisher). Additionally, mouse anti-human primary antibodies that recognize human DRD2 (1:100; B-10; Santa Cruz) or FUT3 (1:100, F3, Thermo Fisher) were used, followed by incubation with an Alexa fluor 647- or 555-conjugated donkey anti-mouse secondary antibody (1:000; Thermo Fisher). In these cases of indirect staining, cells were first blocked with donkey serum (Jackson ImmunoResearch Laboratories) plus human FC block (eBioscience). 7-aminoactinomycin D (Beckman Coulter) was used to discriminate live cells. When appropriate, fluorescence minus one and secondary antibody controls were used to optimize gating strategies for target cell populations.

For whole genome sequencing experiments, leukemic blasts were purified from primary patient MNCs based on CD45-side scatter gating to eliminate healthy human cells. Healthy T cell populations were purified from AML patient MNCs by gating on CD45hiCD3+ cells with low side scatter profiles. Human cells were isolated from xenografts based on CD45+CD33+ gating (AML) or CD45+ gating (healthy). In experiments that involved sub-fractionation of xenografted AML populations, cells were gated on CD45+CD33+, followed by CD34+ versus CD34− sub-gating. Human AML patient BM cells obtained at diagnosis vs. post-chemotherapy were similarly sorted based on CD34+ versus CD34− gates. Human AML patient BM samples obtained at remission were sorted based on CD34+ gating followed by SLC2A2+ vs. SLC2A2− sub-fractionation. Post-sort purities were routinely >95%. FACS sorting was performed using a FACSAria II sorter, and flow cytometry analysis was performed with a LSRII Cytometer (BD), or MACSQuant Analyzer system (Miltenyi Biotec). FACSDiva (BD) and MACSQuantify (Miltenyi Biotec) software was used for data acquisition and FlowJo software (Tree Star) was used for analysis.

### Cell Cycle Analysis

In order to measure active BrdU incorporation as an indicator of S phase cell cycle progression in vivo (Saito et al., 2010), xenografted mice were injected intravenously with 1 mg BrdU, 1 hour prior to sacrifice. Isolated BM cells were stained with cell surface antibodies followed by fixation/permeabilization. Cells were then DNase treated according to protocols outlined in the BD Pharmingen BrdU Flow Kit (#552598). APC-conjugated anti-BrdU was then incubated at a dilution of 1:50 and data were acquired by flow cytometry. In parallel, xenograft BM cells were also analyzed for Ki67 expression to detect active progression through a wider range of cell cycle phases, including late G1, S, G2, and M (Bologna-Molina et al., 2013). Cells were stained with cell surface antibodies and fixable violet dead cell stain (1:10000; L34963; ThermoFisher) prior to fixation in BD Permeabilization/Fixation solution. Intracellular staining was then performed with PE-conjugated anti-Ki67 (1:50; B56; BD Pharmingen) and cells were analyzed by flow cytometry.

Xenograft BM and primary human AML cells were also stained with Hoechst and Pyronin Y to discriminate quiescent (GO) cells from those committed to cell cycle progression. Cells were stained with cell surface antibodies and then incubated with Hoechst 33342 (1:1000; 1 hour at 37° C.; ThermoFisher) and Pyronin Y (0.5 μg/mL; 15 minutes at 37° C.; Sigma-Aldrich) prior to flow cytometry analysis.

### RNA Purification and Gene Expression Profiling

RNA was isolated from human cell populations using a total RNA purification kit (Norgen Biotek) according to the manufacturer's instructions. Xenograft BM samples were FACS-purified to isolate healthy or leukemic human populations prior to RNA extraction. RNA was also isolated from serial samples collected from human AML patients, before and after chemotherapy treatment. These samples were selected based on high leukemic blast frequencies that were comparable between pre-treatment and post-treatment samples. If leukemic blasts did not compose the majority of the mononuclear cells, blast populations were sorted from both pre-treatment and post-treatment cells based on CD45-side scatter profiles (AML #15). Purified RNA was quantified on a Nanodrop 2000 Spectrophotometer (Thermo Scientific), and RNA integrity was assessed by a 2100 Bioanalyzer (Agilent Technologies). RNA was extracted and hybridized to Affymetrix Gene Chip Human Gene 2.0 ST arrays (London Regional Genomics Centre). Output data was normalized using the Robust Multichip Averaging algorithm with Genomics Suite 6.6 software (Partek Inc). Gene expression data for three AML patient-derived xenografts was obtained from publically available datasets (Farge et al., 2017) (GSE97631). Patient-level gene expression data was also obtained from publically accessible data sets for 11 paired diagnosis-relapse samples (Hackl et al., 2015) (GSE66525) and was combined with 4 paired diagnosis-relapse samples from this study (AML #21-24). Batch correction was performed on sources of technical variation (array technologies and/or scan date). Gene set enrichment analysis (GSEA) was performed on normalized expression values of all common gene symbols between samples using GSEA software v2.1.0 (Broad Institute). Functional association networks were identified within differentially expressed gene lists (fold change >1.2 and p<0.05) using the STRING database v10.5. Network visualization was performed using Cytoscape v3.3.0. Druggable gene targets were identified using the Drug Gene Interaction Database (DGIdb v2.22). Pearson's correlation coefficient was used for hierarchical clustering to generate dendograms.

### Whole Genome Sequencing

Genomic DNA was extracted from FACS-purified leukemic blasts from AML Patient #2 in parallel with matched FACS-purified T cells as a healthy genomic control from the same patient. DNA was also extracted from FACS-purified human leukemic cells recovered from the BM of four independent xenografts that were transplanted with cells from the same AML patient and treated with two rounds of AraC in vivo. All DNA extractions were performed using a Qiagen DNeasy kit. PCR-free genomic libraries were constructed for each sample and 150 bp paired-end reads were generated using an Illumina HiSeq X. Sequencing depth was 70-80× for human leukemic samples (de novo patient cells and xenograft-purified cells), and ˜40× for healthy human T-cells. Sequences were aligned to the human reference sequence build GRCh37-lite using bwa-mem version 0.7.6a. Indels and SNVs were called using Strelka version 2.0.7 and SAMtools version 0.1.17. Loss of heterozygosity and copy number alterations were identified using CNAseq version 0.0.6 and APOLLOH version 0.1.1. Subclonal heterogeneity was assessed using Pyclone software (Roth et al., 2014) using 394 high confidence somatic SNVs present in the patient cells and corresponding xenografts. These SNVs were selected based on quality filters (coverage in both leukemic samples and healthy genomic control) and somatic filters (alternative base count in healthy genomic DNA). All leukemic samples were considered equally to discover SNVs for clonal analysis in order to capture any SNVs uniquely arising in xenografts that were absent in de novo patient cells.

### Droplet Digital Polymerase Chain Reaction

Detection of NPM1 c.863_864 insTCTG (COSMIC 17559) was performed on the QX200 Droplet Digital PCR system (Bio-Rad Laboratories, Inc., Hercules, Calif., USA) using TaqMan™ Liquid Biopsy dPCR Assay Hs000000064_rm (Life Technologies, Carlsbad, Calif., USA). The 20 μl reaction mix consisted of 10 μl of 2×ddPCR SuperMix for Probes (Bio-Rad Laboratories), 0.5 μl of the 40× assay, 9.5 μl water and 1 μl of 30-50 ng/μl genomic DNA. The assay was tested by temperature gradient to ensure optimal separation of reference and variant signals. Cycling conditions for the reaction were 95C for 10 min, followed by 45 cycles of 94° C. for 30 s and 60° C. for 1 min, 98° C. for 10 min and finally a 4° C. hold on a Life Technologies Veriti thermal cycler. Data was analyzed using QuantaSoft Analysis Pro software v1.0.596 (Bio-Rad Laboratories).

### Hematology Analysis

Circulating blast counts were measured from human AML patients before and after chemotherapy treatment, by standard complete blood count analysis in the clinic. Murine peripheral blood was collected from the superficial temporal vein and tail vein. Whole blood was then stained with acridine orange to measure white blood cell counts on a Nexcelom Cellometer.

### Quantification and Analysis

Summarized data are represented as mean±standard error (SEM). Statistical comparisons were analyzed using unpaired Student's t-tests (two-tailed), paired t-tests, one-way analysis of variance tests (ANOVAs) followed by Newman-Keuls Multiple Comparison tests, two-way ANOVAs, or Fisher's exact tests. Prism software was used for statistical analysis (version 5.0a; GraphPad), and p<0.05 was considered statistically significant. Any deviations from normal distribution or homogeneity of variances were corrected by log 10 transformation prior to parametric statistical tests. If parametric assumptions could not be met, data were analyzed by Mann-Whitney U tests (FIGS. 5E and 7H) or Kruskal-Wallis tests with Dunn's Multiple Comparison tests. In some cases, different tests were used for independent comparisons within the same figure, based on the distributions of the data sets (e.g., FIG. 1D, Mann-Whitney test was used for Patient #3 G1 phase, and unpaired t-tests were used for all other comparisons; FIG. 1H, unpaired t-test was used for Patient #2 and Fisher's Exact Test was used for Patient #3; FIG. 1J, t-test was used for Patient #3 2×105, and Fisher's Exact Tests were used for all other comparisons. A summary of sample sizes used in xenograft experiments can be found in Table 7.

### Data and Software Availability

Microarray data generated in this study can be accessed at GEO (accession code GSE75086).

### Tables

TABLE 1
Clinical details of AML patient samples

| Patient | | Tissue | |
|---|---|---|---|
| ID | Disease stage | source | CG/Molecular |
| 1 | Diagnosis/post- | BM/BM | Normal |
| | induction | | |
| 2 | Progressed from MDS | PB | inv(3)(q21q26.2), −7 |
| 3 | Diagnosis | PB | del(5) (q22q33), −7 |
| 4 | Multiple over time | BM | Normal/FLT3-ITD |
| 5 | Diagnosis | PB | Normal/FLT3-ITD |
| 6 | Diagnosis | BM | Normal/None detected |
| 7 | Progressed from MDS | PB | 48, XY, +8, +13[8]/46, XY[5]/FLT3-ITD, JAK2 (v617F) neg |
| 8 | Diagnosis | PB | 46, XX, del(5)(q22q35)[cp3]/45-46, |
| | | | idem, del(7)(q32)[cp2]/44-46, |
| | | | idem, t(1; 12)(p13; p13), del(2)(p23)[cp2]/ |
| | | | 42-46, |
| | | | idem, del(3)(p22p24), der(3)inv(3)(p21q21)del(3)(q21), |
| | | | del(7)(q32), add(18)(q21), add(20)(p12)[cp13]/ |
| | | | 44-46, idem, del(1)(p22p32), del(3)(p22p24), del(4)(q21), |
| | | | del(7)(q22q36), del(9)(q22q32), add(12)(q24.1)[cp5] |
| 9 | Diagnosis | BM | 45-46, XY, 1-38 dmin[10]/46, XY[10].nuc ish (MLLx2)[192] |
| 10 | Diagnosis | PB | Normal/NPM1, FLT3-ITD |
| 11 | Diagnosis/post- | PB/BM | Normal |
| | induction | | |
| 12 | Diagnosis | PB | NA/None detected |
| 13 | Diagnosis | PB | NA/NA |
| 14 | Diagnosis | PB | NA/None detected |
| 15 | Diagnosis/post- | PB/PB | 46-47, XX, del(5)(q13q33), del(13)(q12q14), +21, +22[cp26] |
| | induction | | |
| 16 | Diagnosis/post- | BM/BM | 47, XY, +13[24]/46, XY[1] |
| | induction | | |
| 17 | Diagnosis/post- | BM/BM | 46, XY, inv(3)(p12q26), t(11; 15)(q23; q14)[25] |
| | induction | | |
| 18 | Diagnosis/post- | BM/BM | NA (normal FISH) |
| | induction | | |
| 19 | Diagnosis/post- | BM/BM | Normal |
| | induction | | |
| 20 | Diagnosis/post- | BM/BM | 42-46, X, −Y, |
| | induction | | t(2; 15)(q37; q22), dic(5; 17)(q11; p11), +10, add(11)(p15), −17, −18, |
| | | | −21, −22, +2-4mar[cp19]/ |
| | | | 62<3n>, XYY, +1, t(2; 15)(q37; q22), −3, |
| | | | −5, +13, −17, −17, −18, −19, −19, −21, −22[1]/ |
| | | | 80-83 |
| | | | <4n>, XXYY, der(1)t(1; 11)(q32; q13)x2, t(2; 15)(q37; q22)x2, |
| | | | −3, −5, dic(5; 17)(q11; p11), −7, −11, −16, −17, −17, −18, −20, −21, |
| | | | −21, +2mar[cp2]/46, XY[3] |
| 21 | Diagnosis/relapse | PB/PB | Normal/NPM1, FLT3-ITD |
| 22 | Diagnosis/relapse | BM/PB | Add1, −3, del3 (q21), de15 (q13q33), −7, −10, add11, |
| | | | del12 (p11.2p13), add13, add16, t(7; 17)(p13; p13), −18, |
| | | | +21 |
| 23 | Diagnosis/relapse | BM/PB | Normal/NPM1, FLT3-ITD |
| 24 | Diagnosis/relapse | BM/PB | NA/NA |
| 25 | Diagnosis/post- | BM | 46, XX, inv(16)(p13q22) |
| | induction | | |
| 26 | Diagnosis/post- | BM | 46, XX, inv(16)(p13q22)[4]/47, idem, +22[21] |
| | induction | | |
| 27 | Diagnosis/post- | BM | 46, XX, t(8; 21)(q22; q22) |
| | induction | | |
| 28 | Diagnosis/post- | PB/BM | nuc ish(MLLx2)[200]/NPM1 |
| | induction | | |
| 29 | post-induction | BM | Normal/FLT3-ITD |

TABLE 2
LSC quantification upon AraC cytoreduction

| | | | | | Relative LSC |
|---|---|---|---|---|---|
| Patient | | | #Engrafted | Estimated LSC | frequency (Fold |
| ID | Condition | Cell dose | mice | frequency | change) |
| AML#2 | −AraC | 1 × 10\hat{ }6 | 3/3 | >1 in 2 × 10\hat{ }5* | At least 24x |
| AML#2 | −AraC | 2 × 10\hat{ }5 | 3/3 | | reduction |
| AML#2 | +AraC | 1 × 10\hat{ }6 | 0/4 | <1 in 4.8 × 10\hat{ }6** | |
| AML#2 | +AraC | 2 × 10\hat{ }5 | 0/4 | | |
| AML#3 | −AraC | 2 × 10\hat{ }5 | 4/4 | >1 in 3 × 10\hat{ }4* | At least 26x |
| AML#3 | −AraC | 3 × 10\hat{ }4 | 4/4 | | reduction |
| AML#3 | +AraC | 2 × 10\hat{ }5 | 1/4 | 1 in 7.85 × 10\hat{ }5 | |
| AML#3 | +AraC | 3 × 10\hat{ }4 | 0/3 | | |

*Based on positive engraftment in each 2′ mouse at the lowest cell dose tested
**Based on the absence of engraftment across all 2′ mice tested, representing a total of 4.8 × 10\hat{ }6 cells

TABLE 3
LSC quantification at the onset of regeneration

| | | | Estimated | Relative LSC |
|---|---|---|---|---|
| | | # Mice | LSC | frequency |
| Patient ID | Condition | transplanted | frequency | (Fold change) |
| AML #2 | −AraC | 11 | 1 in 102,244 | 1.49× increase |
| AML #2 | +AraC | 11 | 1 in 68,440 | |
| AML #5 | −AraC | 23 | 1 in 478,543 | 2.33× increase |
| AML #5 | +AraC | 20 | 1 in 205,562 | |
| AML #6 | −AraC | 6 | 1 in 8,972,412 | 4.69× increase |
| AML #6 | +AraC | 3 | 1 in 1,911,385 | |

TABLE 4A
Genes preferentially expressed by leukemic regenerating cells.
The genes in bold were commonly upregulated by leukemic regenerating
cells and healthy hematopoietic regenerating cells.

| | | | Fold-Change in | |
|---|---|---|---|---|
| | | | “+AraC” versus | |
| | Transcript | | “−AraC” leukemic | |
| Transcript name | ID | RefSeq ID | xenografts | p value |
| IFNA13 | 17092862 | NM_006900 | 1.901 | 0.020 |
| OR4D10 | 16725091 | NM_001004705 | 1.735 | 0.014 |
| KRT6B | 16764894 | NM_005555 | 1.632 | 0.004 |
| OR1S2 | 16738599 | NM_001004459 | 1.604 | 0.010 |
| FDCSP | 16967465 | NM_152997 | 1.576 | 0.021 |
| ZSCAN5C | 16865860 | ENST00000534327 | 1.533 | 0.008 |
| OR52J3 | 16721223 | NM_001001916 | 1.518 | 0.016 |
| KIR2DS4 | 16865522 | NM_001281971 | 1.486 | 0.024 |
| CSN3 | 16967472 | NM_005212 | 1.470 | 0.012 |
| SLC2A2 | 16961487 | NM_000340 | 1.463 | 0.009 |
| OR52E6 | 16734958 | NM_001005167 | 1.462 | 0.011 |
| OR5AP2 | 16738395 | NM_001002925 | 1.451 | 0.005 |
| SSX4B | 17110576 | NM_001034832 | 1.448 | 0.002 |
| OR6Q1 | 16724989 | NM_001005186 | 1.440 | 0.003 |
| OR10J5 | 16695147 | BC137025 | 1.440 | 0.008 |
| KRTAP21-2 | 16924862 | ENST00000333892 | 1.433 | 0.006 |
| KRTAP9-8 | 16834151 | NM_031963 | 1.424 | 0.008 |
| KRTAP2-2 | 16844581 | NM_{—}033032 | 1.408 | 0.024 |
| ZP4 | 16700989 | NM_{—}021186 | 1.400 | 0.031 |
| MS4A18 | 16725324 | XM_006718756 | 1.392 | 0.004 |
| TXNDC8 | 17097060 | NM_001003936 | 1.388 | 0.049 |
| GAREM | 16854594 | NM_001242409 | 1.386 | 0.020 |
| ADAMTS16 | 16982870 | NM_139056 | 1.382 | 0.047 |
| RP11-500M8.7 | 16747072 | OTTHUMT00000472988 | 1.379 | 0.017 |
| OR5L2 | 16724751 | NM_001004739 | 1.369 | 0.040 |
| ACSS3 | 16754729 | NM_024560 | 1.358 | 0.042 |
| C1QTNF9 | 16773224 | ENST00000382071 | 1.354 | 0.012 |
| TP53TG3 | 16818451 | AK097435 | 1.350 | 0.036 |
| TMEM74B | 16916396 | NM_018354 | 1.349 | 0.027 |
| SYNGR2 | 16838330 | NM_004710 | 1.347 | 0.032 |
| GPR148 | 16885629 | NM_207364 | 1.344 | 0.004 |
| KCNJ10 | 16695262 | NM_002241 | 1.343 | 0.013 |
| OR9Q2 | 16724991 | NM_001005283 | 1.342 | 0.004 |
| SSX7 | 17111093 | NM_173358 | 1.342 | 0.036 |
| ARNT2 | 16803710 | ENST00000533983 | 1.341 | 0.027 |
| OR6P1 | 16695044 | NM_001160325 | 1.335 | 0.006 |
| OR6K2 | 16695111 | BC137022 | 1.329 | 0.023 |
| LYPD4 | 16872626 | NM_{—}173506 | 1.325 | 0.004 |
| SLAMF1 | 16695422 | NM_003037 | 1.323 | 0.003 |
| C9orf57 | 17094865 | NM_001128618 | 1.322 | 0.001 |
| OTOL1 | 16947662 | NM_001080440 | 1.317 | 0.014 |
| OR11H2 | 16789888 | ENST00000556246 | 1.317 | 0.046 |
| FASLG | 16673928 | NM_000639 | 1.314 | 0.008 |
| B3GALT5 | 16922865 | ENST00000380620 | 1.312 | 0.041 |
| GTF2IRD1 | 17047138 | NM_001199207 | 1.311 | 0.008 |
| IFNL1 | 16861961 | NM_172140 | 1.311 | 0.038 |
| BCL11B | 16796590 | NM_001282237 | 1.307 | 0.046 |
| OR2AG1 | 16721519 | NM_001004489 | 1.307 | 0.001 |
| LCE1A | 16671082 | NM_{—}178348 | 1.306 | 0.050 |
| LOC388282 | 16819623 | NM_001278081 | 1.304 | 0.011 |
| LPAR3 | 16688992 | NM_012152 | 1.299 | 0.045 |
| ESPNL | 16893041 | NM_194312 | 1.297 | 0.010 |
| LELP1 | 16671125 | NM_001010857 | 1.296 | 0.047 |
| ZNF793 | 16861563 | NM_001013659 | 1.295 | 0.024 |
| OR5M8 | 16738383 | BC136978 | 1.295 | 0.007 |
| IL18RAP | 16883733 | NM_003853 | 1.295 | 0.041 |
| PARK2 | 17025440 | NM_004562 | 1.294 | 0.014 |
| OR8D4 | 16732790 | NM_001005197 | 1.292 | 0.017 |
| OR6V1 | 17052734 | ENST00000418316 | 1.292 | 0.027 |
| IGSF11 | 16957715 | NM_001015887 | 1.292 | 0.016 |
| MAS1L | 17041490 | ENST00000377127 | 1.289 | 0.008 |
| GPR139 | 16824583 | ENST00000326571 | 1.288 | 0.003 |
| TP53TG3B | 16818481 | NM_{—}001099687 | 1.287 | 0.049 |
| PITX2 | 16979024 | NM_{—}001204397 | 1.285 | 0.017 |
| KRTAP1-3 | 16844568 | NM_030966 | 1.284 | 0.003 |
| SLC36A2 | 17001879 | NM_181776 | 1.283 | 0.003 |
| MOG | 17035008 | ENST00000259891 | 1.283 | 0.019 |
| OR1N1 | 17098118 | NM_012363 | 1.282 | 0.017 |
| CXCL12 | 16713530 | NM_{—}000609 | 1.281 | 0.029 |
| DRD2 | 16744461 | ENST00000540600 | 1.280 | 0.044 |
| CARTPT | 16985943 | NM_004291 | 1.280 | 0.032 |
| LY6G6C | 17039517 | ENST00000383413 | 1.279 | 0.023 |
| SHE | 16693778 | NM_001010846 | 1.279 | 0.027 |
| PAFAH1B3 | 16872767 | NM_001145939 | 1.279 | 0.016 |
| PCLO | 17059249 | NM_033026 | 1.278 | 0.002 |
| MBD3L5 | 16867905 | NM_001136507 | 1.277 | 0.012 |
| C16orf92 | 16817784 | NM_001109660 | 1.276 | 0.007 |
| OPALIN | 16716946 | NM_001040103 | 1.271 | 0.028 |
| PLG | 17014459 | NM_000301 | 1.270 | 0.046 |
| RASGRF2 | 16986777 | NM_006909 | 1.270 | 0.042 |
| GPR1 | 16907572 | NM_001261452 | 1.269 | 0.047 |
| ACCSL | 16723981 | NM_001031854 | 1.269 | 0.011 |
| SOX6 | 16736120 | NM_001145811 | 1.269 | 0.024 |
| FUT3 | 16867572 | NM_000149 | 1.269 | 0.016 |
| PRR29 | 16837055 | NM_001164257 | 1.267 | 0.025 |
| EVPLL | 16831844 | NM_001145127 | 1.267 | 0.027 |
| OR8A1 | 16732846 | ENST00000284287 | 1.265 | 0.044 |
| HEPHL1 | 16730157 | NM_001098672 | 1.265 | 0.026 |
| OMD | 17095882 | NM_005014 | 1.264 | 0.008 |
| OR4E2 | 16781825 | NM_001001912 | 1.262 | 0.004 |
| KRTAP5-1 | 16734281 | NM_001005922 | 1.261 | 0.024 |
| ZNF578 | 16864873 | NM_001099694 | 1.261 | 0.001 |
| KRT25 | 16844430 | NM_181534 | 1.260 | 0.011 |
| MUC3A | 17049607 | NM_005960 | 1.260 | 0.029 |
| PGLYRP4 | 16693393 | NM_020393 | 1.260 | 0.011 |
| SYN2 | 16937725 | uc003bwl.1 | 1.257 | 0.005 |
| TMC7 | 16816386 | NM_001160364 | 1.256 | 0.002 |
| UNC13C | 16801243 | NM_001080534 | 1.255 | 0.018 |
| TEX19 | 16839033 | NM_207459 | 1.254 | 0.006 |
| KRTAP4-7 | 16834124 | NM_033061 | 1.253 | 0.007 |
| ZNF454 | 16993311 | NM_001178089 | 1.253 | 0.030 |
| SMLR1 | 17012556 | NM_001195597 | 1.253 | 0.020 |
| KRT36 | 16844724 | NM_003771 | 1.252 | 0.020 |
| CFHR5 | 16675481 | NM_030787 | 1.251 | 0.038 |
| RGR | 16706757 | ENST00000483771 | 1.251 | 0.044 |
| SHISA4 | 16675874 | ENST00000481699 | 1.250 | 0.021 |
| GRM5 | 16743130 | NM_000842 | 1.250 | 0.042 |
| IFNL3 | 16872181 | ENST00000413851 | 1.249 | 0.019 |
| ADAM18 | 17068266 | NM_014237 | 1.248 | 0.037 |
| LOC101927531 | 16758995 | ENST00000536639 | 1.248 | 0.025 |
| GAGE12C | 17103620 | NM_001098408 | 1.248 | 0.008 |
| TPTE | 16924113 | NM_001290224 | 1.247 | 0.044 |
| PRR32 | 17106816 | NM_001122716 | 1.245 | 0.032 |
| OR6C3 | 16752164 | NM_054104 | 1.245 | 0.016 |
| HTR4 | 17001374 | NM_000870 | 1.244 | 0.015 |
| KCNA10 | 16690735 | NM_005549 | 1.244 | 0.027 |
| C12orf54 | 16750597 | NM_152319 | 1.244 | 0.004 |
| CELA2B | 16659637 | NM_015849 | 1.243 | 0.037 |
| UNC79 | 16787693 | NM_020818 | 1.242 | 0.011 |
| VWC2L | 16890457 | ENST00000312504 | 1.242 | 0.039 |
| NALCN | 16780699 | NM_052867 | 1.241 | 0.001 |
| COL3A1 | 16888610 | NM_000090 | 1.239 | 0.041 |
| RTDR1 | 16932965 | NM_014433 | 1.238 | 0.019 |
| HOPX | 16976177 | uc003hcd.2 | 1.236 | 0.036 |
| TUSC3 | 17065958 | NM_006765 | 1.235 | 0.037 |
| C11orf40 | 16734774 | ENST00000307616 | 1.235 | 0.023 |
| TMEM202 | 16802713 | NM_001080462 | 1.235 | 0.008 |
| ACTL7B | 17096855 | ENST00000374667 | 1.235 | 0.038 |
| OR7C1 | 16869710 | ENST00000248073 | 1.235 | 0.042 |
| VIPR2 | 17065017 | NM_003382 | 1.235 | 0.034 |
| GML | 17073251 | NM_002066 | 1.235 | 0.027 |
| C6orf52 | 17015587 | NM_001145020 | 1.234 | 0.016 |
| BAAT | 17096623 | NM_001127610 | 1.234 | 0.028 |
| BDKRB1 | 16788036 | NM_000710 | 1.234 | 0.042 |
| ST8SIA1 | 16762202 | NM_003034 | 1.233 | 0.029 |
| OSBP2 | 16929015 | NM_001282738 | 1.233 | 0.002 |
| KRTAP25-1 | 16924801 | NM_{—}001128598 | 1.233 | 0.044 |
| C12orf56 | 16767020 | NM_001099676 | 1.232 | 0.033 |
| SPATA3 | 16892015 | ENST00000452881 | 1.232 | 0.025 |
| TMEM249 | 17082578 | NM_001252402 | 1.232 | 0.024 |
| ZNF683 | 16683852 | NM_001114759 | 1.232 | 0.005 |
| OR6C2 | 16752174 | NM_054105 | 1.231 | 0.027 |
| KRTAP9-2 | 16834143 | NM_031961 | 1.230 | 0.026 |
| CSNK1A1L | 16778231 | NM_145203 | 1.230 | 0.036 |
| HTR1B | 17020995 | AK290080 | 1.229 | 0.045 |
| LCNL1 | 17091517 | ENST00000482657 | 1.229 | 0.034 |
| G6PC | 16834525 | NM_000151 | 1.228 | 0.034 |
| C6orf222 | 17018601 | NM_001010903 | 1.227 | 0.037 |
| NIPAL4 | 16991582 | NM_001099287 | 1.227 | 0.026 |
| STATH | 16967386 | BX649104 | 1.226 | 0.005 |
| FN1 | 16908037 | NM_002026 | 1.225 | 0.045 |
| OR51H1P | 16734809 | ENST00000322059 | 1.225 | 0.049 |
| TMPRSS11E | 16967327 | NM_014058 | 1.225 | 0.009 |
| OR11A1 | 17031358 | ENST00000377149 | 1.225 | 0.012 |
| POM121L2 | 17016461 | NM_033482 | 1.224 | 0.022 |
| CTXN3 | 16988781 | NM_001127385 | 1.224 | 0.014 |
| DEFB115 | 16912290 | NM_001037730 | 1.224 | 0.037 |
| WBSCR28 | 17046993 | NM_182504 | 1.224 | 0.026 |
| TWIST2 | 16893143 | NM_057179 | 1.223 | 0.035 |
| OR52D1 | 16721244 | NM_001005163 | 1.222 | 0.039 |
| KRTAP29-1 | 16844636 | NM_001257309 | 1.222 | 0.031 |
| KCNA4 | 16736926 | NM_002233 | 1.221 | 0.027 |
| CLPS | 17018525 | NM_001252597 | 1.221 | 0.012 |
| TMEM252 | 17094674 | NM_153237 | 1.220 | 0.018 |
| CCDC177 | 16794263 | NM_001271507 | 1.220 | 0.023 |
| IFNA17 | 17092838 | NM_021268 | 1.220 | 0.036 |
| LRRC66 | 16975912 | NM_001024611 | 1.219 | 0.048 |
| FGFR2 | 16719025 | NM_000141 | 1.219 | 0.010 |
| VEPH1 | 16960844 | NM_001167911 | 1.218 | 0.017 |
| GFRA3 | 17000428 | NM_001496 | 1.216 | 0.043 |
| ROR2 | 17095712 | ENST00000375715 | 1.216 | 0.007 |
| C3orf70 | 16962272 | NM_001025266 | 1.216 | 0.044 |
| DEFA5 | 17074336 | NM_021010 | 1.215 | 0.045 |
| AADAC | 16947045 | NM_001086 | 1.214 | 0.032 |
| PRR9 | 16671129 | NM_001195571 | 1.213 | 0.005 |
| LYPD6 | 16886448 | ENST00000392854 | 1.213 | 0.013 |
| LRRN1 | 16936925 | XM_005265351 | 1.211 | 0.017 |
| OR8H1 | 16738371 | ENST00000313022 | 1.209 | 0.019 |
| OR52A1 | 16734831 | NM_012375 | 1.209 | 0.026 |
| SPINK9 | 16990796 | ENST00000511717 | 1.207 | 0.033 |
| STMND1 | 17005113 | NM_001190766 | 1.207 | 0.019 |
| PRSS37 | 17063708 | ENST00000419085 | 1.207 | 0.008 |
| ADAMTS12 | 16995047 | NM_030955 | 1.206 | 0.036 |
| NR0B2 | 16683903 | NM_021969 | 1.205 | 0.025 |
| SNAI2 | 17077004 | NM_003068 | 1.205 | 0.040 |
| ATXN3L | 17109146 | NM_001135995 | 1.205 | 0.042 |
| FAM132A | 16680113 | NM_001014980 | 1.204 | 0.024 |
| ARMC3 | 16703182 | NM_001282746 | 1.204 | 0.029 |
| HMGCS2 | 16691627 | NM_005518 | 1.202 | 0.040 |
| HRASLS5 | 16739733 | NM_001146728 | 1.200 | 0.024 |
| KLHL25 | 16812897 | NM_022480 | 1.200 | 0.045 |

TABLE 4B
Pathways enriched within leukemic regenerating cells

| | | | False |
|---|---|---|---|
| | Pathway description (enriched in in “+AraC” | Observed | discovery |
| Pathway ID | versus “−AraC” leukemic xenografts) | gene count | rate (FDR) |
| GO.2000026 | regulation of multicellular organismal development | 13 | 2.62E−06 |
| GO.0007267 | cell-cell signaling | 10 | 3.21E−05 |
| GO.0051952 | regulation of amine transport | 5 | 3.21E−05 |
| GO.0048639 | positive regulation of developmental growth | 6 | 3.28E−05 |
| GO.0007268 | synaptic transmission | 8 | 8.29E−05 |
| GO.0019220 | regulation of phosphate metabolic process | 11 | 8.29E−05 |
| GO.0045595 | regulation of cell differentiation | 11 | 8.29E−05 |
| GO.0050433 | regulation of catecholamine secretion | 4 | 0.000111 |
| GO.0050793 | regulation of developmental process | 12 | 0.000129 |
| GO.0051240 | positive regulation of multicellular organismal | 10 | 0.000231 |
| | process | | |
| GO.1902531 | regulation of intracellular signal transduction | 10 | 0.000264 |
| GO.0051239 | regulation of multicellular organismal process | 12 | 0.000348 |
| GO.0045597 | positive regulation of cell differentiation | 8 | 0.000527 |
| GO.0007187 | G-protein coupled receptor signaling pathway, | 5 | 0.000544 |
| | coupled to cyclic nucleotide second messenger | | |
| GO.1903531 | negative regulation of secretion by cell | 5 | 0.000563 |
| GO.0002029 | desensitization of G-protein coupled receptor | 3 | 0.000572 |
| | protein signaling pathway | | |
| GO.0014059 | regulation of dopamine secretion | 3 | 0.000572 |
| GO.0050767 | regulation of neurogenesis | 7 | 0.000696 |
| GO.0050769 | positive regulation of neurogenesis | 6 | 0.000696 |
| GO.0043408 | regulation of MAPK cascade | 7 | 0.000736 |
| GO.0044093 | positive regulation of molecular function | 10 | 0.00078 |
| GO.0065009 | regulation of molecular function | 12 | 0.00078 |
| GO.0040008 | regulation of growth | 7 | 0.000861 |
| GO.0048585 | negative regulation of response to stimulus | 9 | 0.000922 |
| GO.1901700 | response to oxygen-containing compound | 9 | 0.00097 |
| GO.0051954 | positive regulation of amine transport | 3 | 0.00126 |
| GO.0022008 | neurogenesis | 9 | 0.00136 |
| GO.0043085 | positive regulation of catalytic activity | 9 | 0.00149 |
| GO.0009966 | regulation of signal transduction | 11 | 0.00162 |
| GO.0007205 | protein kinase C-activating G-protein coupled | 3 | 0.00184 |
| | receptor signaling pathway | | |
| GO.0030817 | regulation of cAMP biosynthetic process | 4 | 0.00184 |
| GO.0051051 | negative regulation of transport | 6 | 0.00184 |
| GO.0051094 | positive regulation of developmental process | 8 | 0.00201 |
| GO.0021769 | orbitofrontal cortex development | 2 | 0.00203 |
| GO.0051582 | positive regulation of neurotransmitter uptake | 2 | 0.00203 |
| GO.0008015 | blood circulation | 5 | 0.00253 |
| GO.0031399 | regulation of protein modification process | 9 | 0.00262 |
| GO.0001932 | regulation of protein phosphorylation | 8 | 0.00268 |
| GO.1902533 | positive regulation of intracellular signal | 7 | 0.00268 |
| | transduction | | |
| GO.0030307 | positive regulation of cell growth | 4 | 0.0028 |
| GO.0048755 | branching morphogenesis of a nerve | 2 | 0.00281 |
| GO.0048523 | negative regulation of cellular process | 13 | 0.00293 |
| GO.0022603 | regulation of anatomical structure morphogenesis | 7 | 0.00325 |
| GO.0090278 | negative regulation of peptide hormone secretion | 3 | 0.00325 |
| GO.0048583 | regulation of response to stimulus | 12 | 0.00326 |
| GO.0051050 | positive regulation of transport | 7 | 0.00326 |
| GO.0051966 | regulation of synaptic transmission, glutamatergic | 3 | 0.00326 |
| GO.0030534 | adult behavior | 4 | 0.00327 |
| GO.0050790 | regulation of catalytic activity | 10 | 0.00352 |
| GO.0032270 | positive regulation of cellular protein metabolic | 8 | 0.00402 |
| | process | | |
| GO.0031325 | positive regulation of cellular metabolic process | 11 | 0.00416 |
| GO.0030334 | regulation of cell migration | 6 | 0.00431 |
| GO.0048699 | generation of neurons | 8 | 0.00446 |
| GO.0050805 | negative regulation of synaptic transmission | 3 | 0.00446 |
| GO.0000902 | cell morphogenesis | 7 | 0.00448 |
| GO.0001558 | regulation of cell growth | 5 | 0.00466 |
| GO.0002683 | negative regulation of immune system process | 5 | 0.00479 |
| GO.0045937 | positive regulation of phosphate metabolic process | 7 | 0.00488 |
| GO.0051241 | negative regulation of multicellular organismal | 7 | 0.00501 |
| | process | | |
| GO.0019725 | cellular homeostasis | 6 | 0.00529 |
| GO.0050708 | regulation of protein secretion | 5 | 0.00529 |
| GO.0050796 | regulation of insulin secretion | 4 | 0.00529 |
| GO.0008285 | negative regulation of cell proliferation | 6 | 0.00542 |
| GO.0048522 | positive regulation of cellular process | 13 | 0.00591 |
| GO.0050772 | positive regulation of axonogenesis | 3 | 0.00591 |
| GO.0051967 | negative regulation of synaptic transmission, | 2 | 0.0062 |
| | glutamatergic | | |
| GO.0007626 | locomotory behavior | 4 | 0.00694 |
| GO.0009612 | response to mechanical stimulus | 4 | 0.00694 |
| GO.0031401 | positive regulation of protein modification process | 7 | 0.00694 |
| GO.0040012 | regulation of locomotion | 6 | 0.00694 |
| GO.0042127 | regulation of cell proliferation | 8 | 0.00694 |
| GO.0010976 | positive regulation of neuron projection | 4 | 0.00742 |
| | development | | |
| GO.0043410 | positive regulation of MAPK cascade | 5 | 0.00742 |
| GO.0007200 | phospholipase C-activating G-protein coupled | 3 | 0.00762 |
| | receptor signaling pathway | | |
| GO.0051224 | negative regulation of protein transport | 4 | 0.00764 |
| GO.0051924 | regulation of calcium ion transport | 4 | 0.00801 |
| GO.0045761 | regulation of adenylate cyclase activity | 3 | 0.00817 |
| GO.0051223 | regulation of protein transport | 6 | 0.00817 |
| GO.0061387 | regulation of extent of cell growth | 3 | 0.00817 |
| GO.0002031 | G-protein coupled receptor internalization | 2 | 0.00908 |
| GO.0033605 | positive regulation of catecholamine secretion | 2 | 0.00908 |
| GO.0007165 | signal transduction | 13 | 0.00912 |
| GO.0001501 | skeletal system development | 5 | 0.00926 |
| GO.0000122 | negative regulation of transcription from RNA | 6 | 0.00945 |
| | polymerase II promoter | | |
| GO.0016192 | vesicle-mediated transport | 7 | 0.00945 |
| GO.0043270 | positive regulation of ion transport | 4 | 0.00945 |
| GO.0048869 | cellular developmental process | 11 | 0.00945 |
| GO.0009653 | anatomical structure morphogenesis | 9 | 0.0096 |
| GO.0051482 | positive regulation of cytosolic calcium ion | 2 | 0.00991 |
| | concentration involved in phospholipase C- | | |
| | activating G-protein coupled signaling pathway | | |
| GO.0023057 | negative regulation of signaling | 7 | 0.0103 |
| GO.0040013 | negative regulation of locomotion | 4 | 0.0103 |
| GO.0001963 | synaptic transmission, dopaminergic | 2 | 0.0106 |
| GO.0008344 | adult locomotory behavior | 3 | 0.0106 |
| GO.0010648 | negative regulation of cell communication | 7 | 0.0106 |
| GO.0022604 | regulation of cell morphogenesis | 5 | 0.0106 |
| GO.0023051 | regulation of signaling | 10 | 0.0106 |
| GO.0032102 | negative regulation of response to external | 4 | 0.0106 |
| | stimulus | | |
| GO.0060359 | response to ammonium ion | 3 | 0.0106 |
| GO.0060341 | regulation of cellular localization | 7 | 0.0107 |
| GO.0007631 | feeding behavior | 3 | 0.0112 |
| GO.0006357 | regulation of transcription from RNA polymerase II | 8 | 0.0117 |
| | promoter | | |
| GO.0003008 | system process | 8 | 0.0122 |
| GO.0055080 | cation homeostasis | 5 | 0.0127 |
| GO.0072091 | regulation of stem cell proliferation | 3 | 0.0127 |
| GO.0007210 | serotonin receptor signaling pathway | 2 | 0.0129 |
| GO.0007610 | behavior | 5 | 0.013 |
| GO.0022411 | cellular component disassembly | 5 | 0.013 |
| GO.0031324 | negative regulation of cellular metabolic process | 9 | 0.013 |
| GO.0043066 | negative regulation of apoptotic process | 6 | 0.013 |
| GO.0048878 | chemical homeostasis | 6 | 0.013 |
| GO.0007417 | central nervous system development | 6 | 0.0133 |
| GO.0009893 | positive regulation of metabolic process | 11 | 0.0133 |
| GO.0044700 | single organism signaling | 13 | 0.0133 |
| GO.0051049 | regulation of transport | 8 | 0.0133 |
| GO.0055082 | cellular chemical homeostasis | 5 | 0.0133 |
| GO.0098771 | inorganic ion homeostasis | 5 | 0.0133 |
| GO.0010646 | regulation of cell communication | 10 | 0.0134 |
| GO.0032268 | regulation of cellular protein metabolic process | 9 | 0.0136 |
| GO.0032879 | regulation of localization | 9 | 0.0139 |
| GO.0046887 | positive regulation of hormone secretion | 3 | 0.0142 |
| GO.0050709 | negative regulation of protein secretion | 3 | 0.0142 |
| GO.0044260 | cellular macromolecule metabolic process | 15 | 0.0144 |
| GO.0048518 | positive regulation of biological process | 13 | 0.0144 |
| GO.0045667 | regulation of osteoblast differentiation | 3 | 0.0154 |
| GO.0042592 | homeostatic process | 7 | 0.0156 |
| GO.0045934 | negative regulation of nucleobase-containing | 7 | 0.0156 |
| | compound metabolic process | | |
| GO.0051179 | localization | 12 | 0.0156 |
| GO.0051928 | positive regulation of calcium ion transport | 3 | 0.0156 |
| GO.0051716 | cellular response to stimulus | 14 | 0.0162 |
| GO.1903532 | positive regulation of secretion by cell | 4 | 0.017 |
| GO.0002682 | regulation of immune system process | 7 | 0.0177 |
| GO.0050896 | response to stimulus | 15 | 0.0177 |
| GO.0065008 | regulation of biological quality | 10 | 0.0178 |
| GO.0022617 | extracellular matrix disassembly | 3 | 0.0179 |
| GO.0032147 | activation of protein kinase activity | 4 | 0.0187 |
| GO.0044070 | regulation of anion transport | 3 | 0.0191 |
| GO.0032098 | regulation of appetite | 2 | 0.0194 |
| GO.0043269 | regulation of ion transport | 5 | 0.0194 |
| GO.0031327 | negative regulation of cellular biosynthetic process | 7 | 0.0205 |
| GO.1903530 | regulation of secretion by cell | 5 | 0.0205 |
| GO.0072507 | divalent inorganic cation homeostasis | 4 | 0.0207 |
| GO.0007612 | learning | 3 | 0.0208 |
| GO.0009719 | response to endogenous stimulus | 7 | 0.0208 |
| GO.0010033 | response to organic substance | 9 | 0.021 |
| GO.0001964 | startle response | 2 | 0.0217 |
| GO.0030154 | cell differentiation | 10 | 0.0217 |
| GO.0042417 | dopamine metabolic process | 2 | 0.0228 |
| GO.0023056 | positive regulation of signaling | 7 | 0.023 |
| GO.0022408 | negative regulation of cell-cell adhesion | 3 | 0.0239 |
| GO.0030335 | positive regulation of cell migration | 4 | 0.0249 |
| GO.0071495 | cellular response to endogenous stimulus | 6 | 0.0249 |
| GO.0007399 | nervous system development | 8 | 0.025 |
| GO.0008361 | regulation of cell size | 3 | 0.025 |
| GO.0010837 | regulation of keratinocyte proliferation | 2 | 0.025 |
| GO.0030900 | forebrain development | 4 | 0.025 |
| GO.0032108 | negative regulation of response to nutrient levels | 2 | 0.025 |
| GO.0048169 | regulation of long-term neuronal synaptic plasticity | 2 | 0.025 |
| GO.1902230 | negative regulation of intrinsic apoptotic signaling | 2 | 0.025 |
| | pathway in response to DNA damage | | |
| GO.0070848 | response to growth factor | 5 | 0.0267 |
| GO.0000904 | cell morphogenesis involved in differentiation | 5 | 0.0268 |
| GO.0009968 | negative regulation of signal transduction | 6 | 0.0278 |
| GO.0018149 | peptide cross-linking | 2 | 0.0279 |
| GO.0021884 | forebrain neuron development | 2 | 0.0279 |
| GO.1903792 | negative regulation of anion transport | 2 | 0.0279 |
| GO.0007188 | adenylate cyclase-modulating G-protein coupled | 3 | 0.0288 |
| | receptor signaling pathway | | |
| GO.0032228 | regulation of synaptic transmission, GABAergic | 2 | 0.0294 |
| GO.0007166 | cell surface receptor signaling pathway | 8 | 0.0296 |
| GO.0010604 | positive regulation of macromolecule metabolic | 9 | 0.0296 |
| | process | | |
| GO.0050679 | positive regulation of epithelial cell proliferation | 3 | 0.03 |
| GO.0008217 | regulation of blood pressure | 3 | 0.0315 |
| GO.0045773 | positive regulation of axon extension | 2 | 0.0326 |
| GO.0048514 | blood vessel morphogenesis | 4 | 0.0327 |
| GO.0060322 | head development | 5 | 0.0327 |
| GO.0010647 | positive regulation of cell communication | 7 | 0.0335 |
| GO.0007186 | G-protein coupled receptor signaling pathway | 6 | 0.0336 |
| GO.2001233 | regulation of apoptotic signaling pathway | 4 | 0.0336 |
| GO.0051270 | regulation of cellular component movement | 5 | 0.036 |
| GO.0044708 | single-organism behavior | 4 | 0.0364 |
| GO.0048468 | cell development | 7 | 0.037 |
| GO.0051128 | regulation of cellular component organization | 8 | 0.037 |
| GO.0006810 | transport | 10 | 0.0379 |
| GO.0043549 | regulation of kinase activity | 5 | 0.0385 |
| GO.0030818 | negative regulation of cAMP biosynthetic process | 2 | 0.0391 |
| GO.0030003 | cellular cation homeostasis | 4 | 0.0457 |
| GO.0046676 | negative regulation of insulin secretion | 2 | 0.0469 |
| GO.0044765 | single-organism transport | 9 | 0.0473 |
| GO.0007154 | cell communication | 12 | 0.0487 |
| GO.0010243 | response to organonitrogen compound | 5 | 0.0488 |
| GO.0048731 | system development | 10 | 0.0496 |
| GO.0001763 | morphogenesis of a branching structure | 3 | 0.0497 |

TABLE 4C
Genes preferentially expressed by healthy
hematopoietic regenerating cells (HRCs)

| | | | Fold-Change in | |
|---|---|---|---|---|
| | | | “+AraC” versus | |
| Transcript | Transcript | | “−AraC” healthy | |
| name | ID | RefSeq ID | xenografts | p value |
| XCR1 | 16952868 | ENST00000309285 | 2.438 | 0.035 |
| CPVL | 17056248 | NM_019029 | 2.219 | 0.010 |
| CXCL16 | 16840113 | NM_001100812 | 2.117 | 0.004 |
| C1orf162 | 16668702 | NM_174896 | 1.824 | 0.042 |
| RGS2 | 16675323 | NM_002923 | 1.811 | 0.007 |
| CD1C | 16672323 | ENST00000443761 | 1.804 | 0.034 |
| HMOX1 | 16929562 | ENST00000216117 | 1.776 | 0.036 |
| FPR3 | 16864756 | NM_002030 | 1.750 | 0.041 |
| DAB2 | 16995645 | NM_001244871 | 1.685 | 0.017 |
| TIMP3 | 16929442 | NM_000362 | 1.680 | 0.010 |
| PDK4 | 17059955 | NM_002612 | 1.649 | 0.022 |
| ANPEP | 16813206 | NM_001150 | 1.646 | 0.006 |
| KIAA1598 | 16718719 | NM_001127211 | 1.632 | 0.035 |
| SIGLEC6 | 16874890 | NM_001245 | 1.625 | 0.008 |
| ATF5 | 17126000 | NM_001193646 | 1.622 | 0.026 |
| ENPP1 | 17012632 | NM_006208 | 1.607 | 0.048 |
| CDK15 | 16889530 | ENST00000260967 | 1.601 | 0.048 |
| CST3 | 16917939 | NM_000099 | 1.589 | 0.024 |
| RAB27B | 16852463 | NM_004163 | 1.586 | 0.018 |
| MTRNR2L10 | 17111545 | NM_001190708 | 1.584 | 0.022 |
| CDH1 | 16820486 | NM_004360 | 1.562 | 0.014 |
| ABCA6 | 16848219 | NM_080284 | 1.543 | 0.038 |
| ZNF532 | 16852647 | NM_018181 | 1.526 | 0.041 |
| AXL | 16862439 | NM_001278599 | 1.522 | 0.050 |
| DUSP5 | 16709128 | NM_004419 | 1.513 | 0.049 |
| STH | 16835037 | NM_001007532 | 1.500 | 0.010 |
| LDLRAD3 | 16723680 | NM_174902 | 1.495 | 0.028 |
| GPR97 | 16819563 | NM_170776 | 1.487 | 0.024 |
| PTGS1 | 17088760 | NM_000962 | 1.485 | 0.021 |
| ANKRD42 | 16729611 | NM_182603 | 1.480 | 0.036 |
| HBG1 | 16734862 | ENST00000330597 | 1.473 | 0.042 |
| NEK3 | 16779369 | NM_001146099 | 1.473 | 0.043 |
| PGLYRP3 | 16693383 | NM_052891 | 1.462 | 0.013 |
| ALDH1A1 | 17094893 | NM_000689 | 1.458 | 0.022 |
| OR10S1 | 16745631 | ENST00000531945 | 1.456 | 0.027 |
| CD1B | 16695023 | NM_001764 | 1.454 | 0.004 |
| OR14C36 | 16679797 | NM_001001918 | 1.443 | 0.002 |
| ITGA2B | 16845681 | NM_000419 | 1.441 | 0.015 |
| LCE1A | 16671082 | NM_178348 | 1.440 | 0.021 |
| MRAS | 16946159 | NM_001252092 | 1.435 | 0.013 |
| HOXA13 | 17056192 | NM_000522 | 1.425 | 0.039 |
| HLA-DQA1 | 17033617 | ENST00000474698 | 1.420 | 0.030 |
| ADRB2 | 16990848 | NM_000024 | 1.415 | 0.037 |
| KRTAP9-3 | 16834148 | NM_031962 | 1.415 | 0.009 |
| HLA-DRB4 | 17037192 | NM_021983 | 1.414 | 0.050 |
| PIK3R6 | 16841060 | NM_001010855 | 1.411 | 0.028 |
| ANGPT1 | 17080082 | NM_001146 | 1.411 | 0.027 |
| CDSN | 17028942 | ENST00000259726 | 1.407 | 0.037 |
| KRTAP5-10 | 16728513 | NM_001012710 | 1.406 | 0.023 |
| TNFRSF10B | 17075426 | NM_003842 | 1.401 | 0.010 |
| GFI1B | 17090670 | ENST00000339463 | 1.401 | 0.047 |
| HLA-DQA2 | 17007292 | NM_020056 | 1.400 | 0.037 |
| PTPRJ | 16724633 | NM_002843 | 1.398 | 0.023 |
| ZNF80 | 16957628 | NM_007136 | 1.391 | 0.023 |
| RAB7B | 17126288 | NM_001164522 | 1.390 | 0.021 |
| LOC100507494 | 17117760 | AK090481 | 1.389 | 0.040 |
| PARM1 | 16967875 | NM_015393 | 1.387 | 0.007 |
| FMNL2 | 16886564 | NM_052905 | 1.387 | 0.026 |
| LOC100507537 | 16960701 | ENST00000489090 | 1.385 | 0.012 |
| CD80 | 16957795 | NM_005191 | 1.385 | 0.028 |
| PRTFDC1 | 16712576 | NM_001282786 | 1.381 | 0.005 |
| OR7D4 | 16868358 | NM_001005191 | 1.379 | 0.017 |
| HLA-DRB3 | 17027082 | ENST00000426847 | 1.379 | 0.018 |
| OR10G9 | 16732799 | NM_001001953 | 1.376 | 0.011 |
| HLA-DQB1 | 17039923 | NM_001243962 | 1.373 | 0.016 |
| C1orf189 | 16693755 | NM_001010979 | 1.367 | 0.017 |
| PTPRO | 16748711 | NM_030670 | 1.367 | 0.047 |
| LOC93432 | 17052538 | NM_001293626 | 1.361 | 0.039 |
| OR1E2 | 16839692 | NM_003554 | 1.361 | 0.003 |
| LINGO4 | 16693219 | NM_001004432 | 1.358 | 0.018 |
| GHR | 16984365 | NM_000163 | 1.357 | 0.015 |
| HHLA2 | 16943656 | NM_001282556 | 1.348 | 0.015 |
| HERPUD1 | 16819325 | NM_001010989 | 1.341 | 0.011 |
| SLC12A5 | 16914414 | NM_001134771 | 1.340 | 0.004 |
| C8orf46 | 17069577 | ENST00000482608 | 1.340 | 0.004 |
| TAGAP | 17025230 | NM_054114 | 1.339 | 0.044 |
| PTPN20B | 16713897 | ENST00000508357 | 1.339 | 0.033 |
| TMEM40 | 16950877 | NM_001284406 | 1.337 | 0.002 |
| SYTL4 | 17112623 | NM_001129896 | 1.334 | 0.005 |
| KRTAP4-8 | 16844591 | NM_031960 | 1.334 | 0.045 |
| PHKG1 | 17057966 | NM_001258459 | 1.332 | 0.024 |
| CYP7B1 | 17077723 | NM_004820 | 1.331 | 0.036 |
| MAP7 | 17024053 | NM_001198608 | 1.329 | 0.022 |
| TBC1D12 | 16707673 | NM_015188 | 1.327 | 0.015 |
| DGAT2 | 16729168 | ENST00000603276 | 1.327 | 0.010 |
| A2M | 16761012 | NM_000014 | 1.326 | 0.010 |
| GLYAT | 16738646 | NM_005838 | 1.324 | 0.034 |
| FGB | 16971643 | NM_005141 | 1.323 | 0.037 |
| ANKEF1 | 16911394 | NM_022096 | 1.322 | 0.012 |
| SERINC1 | 17023239 | NM_020755 | 1.320 | 0.038 |
| NPL | 16674742 | NM_001200052 | 1.320 | 0.017 |
| SPRED1 | 16799231 | NM_152594 | 1.319 | 0.041 |
| ACVR1B | 16751401 | NM_004302 | 1.316 | 0.042 |
| B3GNT9 | 16827245 | NM_033309 | 1.316 | 0.043 |
| LHFPL2 | 16997503 | NM_005779 | 1.314 | 0.005 |
| CXorf57 | 17105914 | NM_018015 | 1.308 | 0.024 |
| MAGEB6 | 17102285 | NM_173523 | 1.308 | 0.046 |
| PLEKHG3 | 16785410 | NM_015549 | 1.307 | 0.048 |
| CCND1 | 16728261 | NM_053056 | 1.306 | 0.006 |
| HLA-DOB | 17017935 | NM_002120 | 1.305 | 0.026 |
| ITIH5 | 16711598 | NM_001001851 | 1.305 | 0.012 |
| CTTNBP2 | 17062163 | NM_033427 | 1.302 | 0.012 |
| C6orf25 | 17040851 | NM_138277 | 1.301 | 0.002 |
| ZNF521 | 16854360 | NM_015461 | 1.300 | 0.029 |
| TANC1 | 16886818 | NM_001145909 | 1.299 | 0.007 |
| KRTAP2-2 | 16844581 | NM_033032 | 1.299 | 0.043 |
| BCL6 | 16962584 | NM_001706 | 1.298 | 0.041 |
| MYOM3 | 16683493 | NM_152372 | 1.297 | 0.004 |
| ASIC2 | 16843273 | NM_001094 | 1.297 | 0.003 |
| RP11-22P4.1 | 16723120 | OTTHUMT00000388387 | 1.296 | 0.003 |
| KRT15 | 16844752 | ENST00000254043 | 1.295 | 0.011 |
| KCNK2 | 16677451 | NM_001017424 | 1.295 | 0.011 |
| TNNC2 | 16919663 | ENST00000372555 | 1.294 | 0.003 |
| DAB1 | 16687799 | NM_021080 | 1.294 | 0.028 |
| KCNK6 | 16861647 | NM_004823 | 1.293 | 0.036 |
| ACSS2 | 16912975 | NM_001076552 | 1.293 | 0.029 |
| RBMXL3 | 17106345 | NM_001145346 | 1.293 | 0.007 |
| FAM187B | 16871339 | NM_152481 | 1.292 | 0.015 |
| PTK2 | 17081737 | XM_006716606 | 1.292 | 0.003 |
| CXCL12 | 16713530 | NM_000609 | 1.292 | 0.036 |
| LIPI | 16924192 | NM_198996 | 1.292 | 0.020 |
| ADCY1 | 17045806 | NM_021116 | 1.291 | 0.019 |
| RUNX1T1 | 17079037 | NM_001198625 | 1.290 | 0.033 |
| C1orf54 | 16670469 | NM_024579 | 1.289 | 0.000 |
| VASH1 | 16786801 | NM_014909 | 1.289 | 0.019 |
| GPT | 17073890 | NM_005309 | 1.288 | 0.008 |
| OR11L1 | 16701599 | NM_001001959 | 1.287 | 0.031 |
| C11orf87 | 16730967 | NM_207645 | 1.286 | 0.030 |
| GPR87 | 16960567 | NM_023915 | 1.286 | 0.040 |
| PDGFC | 16980946 | NM_016205 | 1.285 | 0.012 |
| HLA-DRB1 | 17034714 | XM_006710243 | 1.284 | 0.012 |
| NEO1 | 16802795 | NM_001172623 | 1.283 | 0.031 |
| FAH | 16803680 | NM_000137 | 1.283 | 0.006 |
| RASA4 | 17061127 | NM_001079877 | 1.282 | 0.033 |
| FANK1 | 16710453 | NM_145235 | 1.282 | 0.009 |
| PKIB | 17012182 | NM_001270393 | 1.281 | 0.042 |
| CD1A | 16672315 | NM_001763 | 1.281 | 0.044 |
| CD300LG | 16834672 | NM_145273 | 1.281 | 0.021 |
| LMNA | 16671914 | NM_001257374 | 1.279 | 0.046 |
| HLA-DQB2 | 17042433 | ENST00000415137 | 1.278 | 0.027 |
| SNX3 | 17022349 | NM_003795 | 1.278 | 0.005 |
| FAM135B | 17081546 | NM_015912 | 1.277 | 0.038 |
| TEX35 | 16674240 | NM_032126 | 1.276 | 0.026 |
| AOC1 | 17053436 | ENST00000467291 | 1.276 | 0.034 |
| TNC | 17097661 | NM_002160 | 1.275 | 0.001 |
| NRP2 | 16889879 | NM_003872 | 1.275 | 0.031 |
| MAP3K8 | 16703659 | XM_006717377 | 1.272 | 0.023 |
| NTRK2 | 17086386 | NM_006180 | 1.271 | 0.027 |
| LOC643797 | 17117657 | AY358245 | 1.271 | 0.045 |
| SALL3 | 16853225 | NM_171999 | 1.269 | 0.002 |
| CLNK | 16974325 | NM_052964 | 1.269 | 0.023 |
| LYPD4 | 16872626 | NM_173506 | 1.268 | 0.003 |
| TMCC2 | 16676437 | NM_014858 | 1.268 | 0.001 |
| CLCNKA | 16659794 | ENST00000464764 | 1.268 | 0.024 |
| TFDP3 | 17114266 | NM_016521 | 1.266 | 0.033 |
| LRRC10 | 16767369 | NM_201550 | 1.265 | 0.008 |
| ARMCX2 | 17112799 | NM_014782 | 1.265 | 0.019 |
| NCR3 | 17041868 | NM_001145466 | 1.264 | 0.022 |
| GOLGA8M | 16806409 | NM_001282468 | 1.263 | 0.027 |
| CGB | 16874187 | NM_000737 | 1.262 | 0.036 |
| GCNT3 | 16801604 | NM_004751 | 1.260 | 0.005 |
| MAS1L | 17041490 | ENST00000377127 | 1.259 | 0.015 |
| SMPDL3B | 16661508 | NM_001009568 | 1.259 | 0.012 |
| SCGB1A1 | 16725871 | NM_003357 | 1.259 | 0.004 |
| PTPN1 | 16914844 | NM_001278618 | 1.259 | 0.030 |
| IL27 | 16825365 | NM_145659 | 1.259 | 0.027 |
| SNAP23 | 16800095 | NM_003825 | 1.258 | 0.037 |
| MPEG1 | 16738694 | NM_001039396 | 1.257 | 0.025 |
| SLC25A53 | 17113047 | NM_001012755 | 1.256 | 0.029 |
| L00100505710 | 16949085 | XM_006713836 | 1.254 | 0.006 |
| CLDN18 | 16946107 | ENST00000183605 | 1.254 | 0.044 |
| C16orf89 | 16823704 | NM_152459 | 1.252 | 0.001 |
| ZBTB47 | 16939703 | NM_145166 | 1.252 | 0.026 |
| OCA2 | 16806249 | NM_000275 | 1.251 | 0.002 |
| IL4R | 16817254 | NM_000418 | 1.251 | 0.013 |
| ZNF474 | 16988462 | NM_207317 | 1.250 | 0.032 |
| FAT4 | 16970465 | NM_001291285 | 1.250 | 0.009 |
| TBC1D9 | 16980096 | NM_015130 | 1.250 | 0.025 |
| KRTAP2-1 | 16844578 | NM_001123387 | 1.249 | 0.000 |
| DFNB59 | 16888157 | NM_001042702 | 1.249 | 0.007 |
| CTTNBP2NL | 16668772 | NM_018704 | 1.246 | 0.046 |
| MYRF | 16725692 | NM_001127392 | 1.246 | 0.018 |
| LIF | 16933760 | NM_001257135 | 1.245 | 0.013 |
| SIGLEC11 | 17122174 | NM_001135163 | 1.244 | 0.032 |
| LRFN5 | 16783739 | NM_152447 | 1.244 | 0.008 |
| KCNJ3 | 16886656 | ENST00000295101 | 1.244 | 0.043 |
| SKIDA1 | 16712442 | NM_207371 | 1.243 | 0.021 |
| HTR7 | 16716469 | NM_019859 | 1.243 | 0.033 |
| LEP | 17051152 | NM_000230 | 1.243 | 0.027 |
| CCDC37 | 16944991 | XM_005247431 | 1.242 | 0.019 |
| TREM1 | 17019056 | NM_018643 | 1.241 | 0.041 |
| MARVELD2 | 16985688 | XM_005276758 | 1.241 | 0.039 |
| TAS1R3 | 16657737 | NM_152228 | 1.240 | 0.010 |
| C2orf80 | 16907743 | NM_001099334 | 1.240 | 0.010 |
| CLEC19A | 16816439 | BX640722 | 1.239 | 0.026 |
| FAM71A | 16699091 | AK097437 | 1.239 | 0.042 |
| GDF5 | 16918722 | ENST00000374372 | 1.239 | 0.008 |
| SLC45A3 | 16698521 | XM_005245560 | 1.239 | 0.048 |
| POM121L12 | 17046091 | NM_182595 | 1.238 | 0.046 |
| GTSF1L | 16919393 | NM_001008901 | 1.237 | 0.041 |
| OGN | 17095870 | NM_014057 | 1.237 | 0.045 |
| IFIT1B | 16707192 | NM_001010987 | 1.236 | 0.013 |
| GPRC5A | 16748529 | NM_003979 | 1.236 | 0.007 |
| CHST4 | 16820873 | NM_001166395 | 1.235 | 0.025 |
| NRSN2 | 16910601 | XM_006723630 | 1.235 | 0.001 |
| XKRX | 17112675 | NM_212559 | 1.235 | 0.042 |
| MMP16 | 17078870 | NM_005941 | 1.235 | 0.031 |
| TBX20 | 17120818 | NM_001077653 | 1.235 | 0.003 |
| MYO1D | 16843241 | NM_015194 | 1.234 | 0.024 |
| GSG1L | 16825252 | NM_001109763 | 1.232 | 0.016 |
| TSPAN10 | 16838841 | NM_001290212 | 1.232 | 0.028 |
| SV2B | 16805124 | NM_014848 | 1.232 | 0.001 |
| CYP4A22 | 16664421 | NM_001010969 | 1.231 | 0.035 |
| ADAM7 | 17066980 | NM_003817 | 1.231 | 0.023 |
| IL17RD | 16955324 | uc010hna.3 | 1.231 | 0.004 |
| PRR25 | 16814565 | NM_001013638 | 1.231 | 0.012 |
| CXorf36 | 17110352 | NM_176819 | 1.231 | 0.047 |
| SNTB1 | 17080630 | NM_021021 | 1.231 | 0.047 |
| DCAF4 | 16786104 | NM_001163508 | 1.231 | 0.004 |
| RP11- | 17074887 | OTTHUMT00000384399 | 1.231 | 0.016 |
| 145O15.3 | | | | |
| FBXO16 | 17075852 | NM_001258211 | 1.230 | 0.042 |
| ZBTB8B | 16662113 | NM_001145720 | 1.230 | 0.037 |
| ROPN1L | 16983236 | NM_031916 | 1.230 | 0.009 |
| GOLM1 | 17095423 | NM_177937 | 1.229 | 0.042 |
| MID2 | 17106051 | NM_012216 | 1.229 | 0.013 |
| KCND3 | 16690908 | NM_004980 | 1.227 | 0.036 |
| NRIP3 | 16735545 | NM_020645 | 1.227 | 0.014 |
| OR10G3 | 16790431 | NM_001005465 | 1.227 | 0.044 |
| SPATA31C1 | 17086585 | NM_001145124 | 1.226 | 0.035 |
| ZNF503 | 16715765 | NM_032772 | 1.225 | 0.033 |
| PDE6B | 16963744 | NM_000283 | 1.225 | 0.017 |
| DSC3 | 16854466 | NM_001941 | 1.224 | 0.015 |
| PTPRH | 16875656 | NM_001161440 | 1.224 | 0.031 |
| CA9 | 17084723 | NM_001216 | 1.224 | 0.032 |
| CYSTM1 | 16989977 | NM_032412 | 1.223 | 0.004 |
| CAMSAP2 | 16675673 | ENST00000413307 | 1.223 | 0.036 |
| GSX1 | 16773541 | NM_145657 | 1.223 | 0.002 |
| LY6G6F | 17035542 | NM_001003693 | 1.222 | 0.037 |
| ZP4 | 16700989 | NM_021186 | 1.222 | 0.010 |
| BEND2 | 17109447 | NM_001184767 | 1.222 | 0.028 |
| SLC22A18 | 16720959 | XM_006725127 | 1.222 | 0.020 |
| KRTAP4-2 | 16844622 | NM_033062 | 1.221 | 0.021 |
| PTPN6 | 16747623 | NM_002831 | 1.221 | 0.043 |
| CAPN6 | 17113362 | NM_014289 | 1.220 | 0.042 |
| C6orf15 | 17036418 | NM_014070 | 1.220 | 0.035 |
| RBPMS | 17067566 | NM_001008710 | 1.220 | 0.024 |
| TP53TG3B | 16818481 | NM_001099687 | 1.220 | 0.018 |
| ABTB2 | 16737260 | NM_145804 | 1.220 | 0.009 |
| OR10A4 | 16721529 | NM_207186 | 1.219 | 0.046 |
| MCC | 16998906 | NM_001085377 | 1.219 | 0.016 |
| CFH | 16675398 | NM_000186 | 1.218 | 0.023 |
| DLC1 | 17074848 | NM_001164271 | 1.218 | 0.011 |
| NUDT8 | 16741113 | NM_001243750 | 1.217 | 0.048 |
| LOC339166 | 16830152 | NR_040000 | 1.217 | 0.016 |
| NLRP13 | 16875836 | NM_176810 | 1.217 | 0.004 |
| INHBB | 16885135 | NM_002193 | 1.216 | 0.049 |
| IL5RA | 16950216 | NM_000564 | 1.215 | 0.013 |
| IDO2 | 17068319 | NM_194294 | 1.215 | 0.009 |
| DAPP1 | 16969229 | NM_014395 | 1.215 | 0.026 |
| GPRC5C | 16837571 | NM_018653 | 1.214 | 0.034 |
| PITX2 | 16979024 | NM_001204397 | 1.214 | 0.034 |
| SLC6A20 | 16952797 | NM_020208 | 1.214 | 0.032 |
| CEP152 | 16800867 | BC029603 | 1.213 | 0.030 |
| MITF | 16942576 | NM_000248 | 1.212 | 0.023 |
| SNCAIP | 16988477 | uc003ksx.1 | 1.212 | 0.046 |
| TAF13 | 16690511 | NM_005645 | 1.212 | 0.009 |
| ATP6AP1L | 16986866 | NM_001017971 | 1.212 | 0.023 |
| ATP2B1 | 16768341 | NM_001001323 | 1.212 | 0.039 |
| ATP13A5 | 16962763 | NM_198505 | 1.211 | 0.004 |
| PGF | 16794846 | NM_001207012 | 1.211 | 0.001 |
| RELB | 16863168 | NM_006509 | 1.211 | 0.002 |
| MAP1LC3B2 | 16757616 | NM_001085481 | 1.211 | 0.005 |
| KRTAP25-1 | 16924801 | NM_001128598 | 1.211 | 0.034 |
| IFNGR2 | 16922275 | NM_005534 | 1.211 | 0.012 |
| PRR15L | 16846157 | NM_024320 | 1.210 | 0.026 |
| TRPV3 | 16839710 | NM_001258205 | 1.210 | 0.004 |
| TTR | 16851786 | NM_000371 | 1.210 | 0.017 |
| PTCHD1 | 17102104 | ENST00000379361 | 1.209 | 0.043 |
| ALKBH2 | 16769868 | NM_001145374 | 1.209 | 0.037 |
| ADAMTSL1 | 17083793 | NM_001040272 | 1.209 | 0.029 |
| CDH23 | 16705844 | NM_001171930 | 1.209 | 0.012 |
| SMOX | 16911108 | NM_001270691 | 1.208 | 0.015 |
| C10orf35 | 16705641 | NM_145306 | 1.208 | 0.044 |
| OR2K2 | 17097150 | NM_205859 | 1.208 | 0.014 |
| NHLH2 | 16691350 | NM_005599 | 1.207 | 0.001 |
| NIPAL2 | 17079448 | NM_024759 | 1.207 | 0.017 |
| ZNF300 | 17001747 | NM_001172831 | 1.207 | 0.041 |
| FERMT2 | 16793067 | NM_001134999 | 1.207 | 0.001 |
| GAPDHS | 16861033 | NM_014364 | 1.206 | 0.011 |
| PRAMEF20 | 16659428 | NM_001099852 | 1.205 | 0.009 |
| THPO | 16962246 | NM_000460 | 1.205 | 0.000 |
| LRRTM4 | 16899461 | NM_001134745 | 1.205 | 0.007 |
| PDE1C | 17056426 | NM_001191057 | 1.205 | 0.048 |
| RAB30 | 16742814 | NM_001286059 | 1.204 | 0.048 |
| SARAF | 17076009 | NM_016127 | 1.204 | 0.011 |
| KRT23 | 16844509 | NM_001282433 | 1.203 | 0.030 |
| OR1M1 | 16857946 | NM_001004456 | 1.203 | 0.018 |
| NUDT2 | 17084439 | NM_001161 | 1.203 | 0.010 |
| RUNDC3B | 17047946 | NM_138290 | 1.202 | 0.021 |
| MS4A15 | 16725334 | NM_152717 | 1.202 | 0.011 |
| TSPO2 | 17008397 | NM_001010873 | 1.202 | 0.017 |
| HLA-DRA | 17041225 | NM_019111 | 1.201 | 0.017 |
| FSTL3 | 16856232 | NM_005860 | 1.201 | 0.034 |
| C17orf96 | 16843981 | NM_001130677 | 1.201 | 0.001 |
| MRGPRG | 16734614 | NM_001164377 | 1.201 | 0.013 |
| GLIS3 | 17092081 | NM_001042413 | 1.201 | 0.049 |
| RAB41 | 17104471 | NM_001032726 | 1.201 | 0.014 |
| WWC2 | 16972710 | NM_024949 | 1.201 | 0.049 |
| PSORS1C1 | 17030550 | ENST00000420214 | 1.200 | 0.007 |
| OR7A5 | 16869713 | NM_017506 | 1.200 | 0.029 |

TABLE 4D
Pathways enriched within healthy hematopoietic regenerating cells (HRCs)

| | | | False |
|---|---|---|---|
| | | Observed | discovery |
| | Pathway description (enriched in in “+AraC” | gene | rate |
| Pathway ID | versus “−AraC” healthy xenografts) | count | (FDR) |
| GO.0070887 | cellular response to chemical stimulus | 16 | 7.18E−06 |
| GO.0071310 | cellular response to organic substance | 14 | 3.11E−05 |
| GO.0007162 | negative regulation of cell adhesion | 7 | 3.26E−05 |
| GO.0007154 | cell communication | 19 | 0.000193 |
| GO.0007155 | cell adhesion | 10 | 0.000193 |
| GO.0044700 | single organism signaling | 19 | 0.000193 |
| GO.0010033 | response to organic substance | 14 | 0.000227 |
| GO.0071345 | cellular response to cytokine stimulus | 8 | 0.000227 |
| GO.0002682 | regulation of immune system process | 11 | 0.000234 |
| GO.0051240 | positive regulation of multicellular organismal | 11 | 0.000234 |
| | process | | |
| GO.0019221 | cytokine-mediated signaling pathway | 7 | 0.00035 |
| GO.0009966 | regulation of signal transduction | 13 | 0.000903 |
| GO.0050878 | regulation of body fluid levels | 8 | 0.000903 |
| GO.0007165 | signal transduction | 17 | 0.00117 |
| GO.0048585 | negative regulation of response to stimulus | 10 | 0.00117 |
| GO.0051716 | cellular response to stimulus | 19 | 0.00117 |
| GO.0010810 | regulation of cell-substrate adhesion | 5 | 0.00137 |
| GO.0048731 | system development | 15 | 0.00139 |
| GO.0009719 | response to endogenous stimulus | 10 | 0.0018 |
| GO.0010604 | positive regulation of macromolecule metabolic | 13 | 0.0018 |
| | process | | |
| GO.0030155 | regulation of cell adhesion | 7 | 0.00198 |
| GO.0030198 | extracellular matrix organization | 6 | 0.00198 |
| GO.0032879 | regulation of localization | 12 | 0.00198 |
| GO.0042127 | regulation of cell proliferation | 10 | 0.00198 |
| GO.0042221 | response to chemical | 15 | 0.00198 |
| GO.0048666 | neuron development | 8 | 0.00198 |
| GO.0051094 | positive regulation of developmental process | 9 | 0.00198 |
| GO.0002698 | negative regulation of immune effector process | 4 | 0.00212 |
| GO.0060396 | growth hormone receptor signaling pathway | 3 | 0.00212 |
| GO.0009725 | response to hormone | 8 | 0.0023 |
| GO.0051239 | regulation of multicellular organismal process | 12 | 0.0023 |
| GO.0071378 | cellular response to growth hormone stimulus | 3 | 0.0023 |
| GO.0009888 | tissue development | 10 | 0.00245 |
| GO.0006950 | response to stress | 14 | 0.00292 |
| GO.0007399 | nervous system development | 11 | 0.00292 |
| GO.0030168 | platelet activation | 5 | 0.00342 |
| GO.0043410 | positive regulation of MAPK cascade | 6 | 0.00342 |
| GO.0007166 | cell surface receptor signaling pathway | 11 | 0.00362 |
| GO.0050777 | negative regulation of immune response | 4 | 0.00362 |
| GO.0048856 | anatomical structure development | 15 | 0.00398 |
| GO.0051241 | negative regulation of multicellular organismal | 8 | 0.00403 |
| | process | | |
| GO.0030182 | neuron differentiation | 8 | 0.00406 |
| GO.0044707 | single-multicellular organism process | 17 | 0.00435 |
| GO.0048699 | generation of neurons | 9 | 0.00435 |
| GO.0030154 | cell differentiation | 13 | 0.00463 |
| GO.0051270 | regulation of cellular component movement | 7 | 0.00506 |
| GO.0048513 | organ development | 12 | 0.00547 |
| GO.0051223 | regulation of protein transport | 7 | 0.00547 |
| GO.1902531 | regulation of intracellular signal transduction | 9 | 0.00547 |
| GO.0031401 | positive regulation of protein modification process | 8 | 0.00557 |
| GO.0022408 | negative regulation of cell-cell adhesion | 4 | 0.00563 |
| GO.0009611 | response to wounding | 7 | 0.00626 |
| GO.0001817 | regulation of cytokine production | 6 | 0.00739 |
| GO.0006468 | protein phosphorylation | 7 | 0.00767 |
| GO.0009605 | response to external stimulus | 10 | 0.00767 |
| GO.0010812 | negative regulation of cell-substrate adhesion | 3 | 0.00767 |
| GO.0031325 | positive regulation of cellular metabolic process | 12 | 0.00767 |
| GO.0048583 | regulation of response to stimulus | 13 | 0.00767 |
| GO.0050776 | regulation of immune response | 7 | 0.00767 |
| GO.0007596 | blood coagulation | 6 | 0.00782 |
| GO.0031589 | cell-substrate adhesion | 4 | 0.00782 |
| GO.1903706 | regulation of hemopoiesis | 5 | 0.00782 |
| GO.0002684 | positive regulation of immune system process | 7 | 0.00786 |
| GO.0018108 | peptidyl-tyrosine phosphorylation | 4 | 0.00786 |
| GO.0007259 | JAK-STAT cascade | 3 | 0.00796 |
| GO.0050731 | positive regulation of peptidyl-tyrosine | 4 | 0.00796 |
| | phosphorylation | | |
| GO.1903708 | positive regulation of hemopoiesis | 4 | 0.00806 |
| GO.2000026 | regulation of multicellular organismal | 9 | 0.00818 |
| | development | | |
| GO.0022407 | regulation of cell-cell adhesion | 5 | 0.00891 |
| GO.0051093 | negative regulation of developmental process | 7 | 0.00917 |
| GO.0022603 | regulation of anatomical structure morphogenesis | 7 | 0.00952 |
| GO.0045623 | negative regulation of T-helper cell differentiation | 2 | 0.00952 |
| GO.0031399 | regulation of protein modification process | 9 | 0.00982 |
| GO.0046903 | secretion | 6 | 0.00999 |
| GO.0007275 | multicellular organismal development | 14 | 0.0102 |
| GO.0048468 | cell development | 9 | 0.0106 |
| GO.0051272 | positive regulation of cellular component | 5 | 0.0106 |
| | movement | | |
| GO.0002683 | negative regulation of immune system process | 5 | 0.0109 |
| GO.0044320 | cellular response to leptin stimulus | 2 | 0.0109 |
| GO.0009653 | anatomical structure morphogenesis | 10 | 0.0124 |
| GO.0045628 | regulation of T-helper 2 cell differentiation | 2 | 0.0124 |
| GO.0050708 | regulation of protein secretion | 5 | 0.0124 |
| GO.0050793 | regulation of developmental process | 10 | 0.0124 |
| GO.0001775 | cell activation | 6 | 0.0127 |
| GO.0006935 | chemotaxis | 6 | 0.0128 |
| GO.0070372 | regulation of ERK1 and ERK2 cascade | 4 | 0.014 |
| GO.0007411 | axon guidance | 5 | 0.0143 |
| GO.0045937 | positive regulation of phosphate metabolic | 7 | 0.0145 |
| | process | | |
| GO.0045596 | negative regulation of cell differentiation | 6 | 0.0152 |
| GO.0031175 | neuron projection development | 6 | 0.0153 |
| GO.0048522 | positive regulation of cellular process | 14 | 0.0154 |
| GO.0060429 | epithelium development | 7 | 0.0154 |
| GO.0048646 | anatomical structure formation involved in | 7 | 0.0162 |
| | morphogenesis | | |
| GO.0051897 | positive regulation of protein kinase B signaling | 3 | 0.0162 |
| GO.0045639 | positive regulation of myeloid cell differentiation | 3 | 0.0173 |
| GO.0042060 | wound healing | 6 | 0.0175 |
| GO.0006796 | phosphate-containing compound metabolic | 9 | 0.0181 |
| | process | | |
| GO.0040012 | regulation of locomotion | 6 | 0.0181 |
| GO.0071495 | cellular response to endogenous stimulus | 7 | 0.0181 |
| GO.1902106 | negative regulation of leukocyte differentiation | 3 | 0.0181 |
| GO.0001952 | regulation of cell-matrix adhesion | 3 | 0.0185 |
| GO.0032268 | regulation of cellular protein metabolic process | 10 | 0.0185 |
| GO.0033197 | response to vitamin E | 2 | 0.0185 |
| GO.0035024 | negative regulation of Rho protein signal | 2 | 0.0185 |
| | transduction | | |
| GO.0002576 | platelet degranulation | 3 | 0.0195 |
| GO.0002700 | regulation of production of molecular mediator of | 3 | 0.0195 |
| | immune response | | |
| GO.0002009 | morphogenesis of an epithelium | 5 | 0.0197 |
| GO.0002719 | negative regulation of cytokine production | 2 | 0.0253 |
| | involved in immune response | | |
| GO.0030728 | ovulation | 2 | 0.0253 |
| GO.0044767 | single-organism developmental process | 14 | 0.0263 |
| GO.0060255 | regulation of macromolecule metabolic process | 15 | 0.0275 |
| GO.0019220 | regulation of phosphate metabolic process | 8 | 0.028 |
| GO.0010594 | regulation of endothelial cell migration | 3 | 0.0285 |
| GO.0033993 | response to lipid | 6 | 0.0295 |
| GO.0014070 | response to organic cyclic compound | 6 | 0.0316 |
| GO.0001934 | positive regulation of protein phosphorylation | 6 | 0.0317 |
| GO.0006071 | glycerol metabolic process | 2 | 0.0317 |
| GO.0008284 | positive regulation of cell proliferation | 6 | 0.0317 |
| GO.0045597 | positive regulation of cell differentiation | 6 | 0.0317 |
| GO.0050678 | regulation of epithelial cell proliferation | 4 | 0.0317 |
| GO.0061564 | axon development | 5 | 0.0317 |
| GO.0065008 | regulation of biological quality | 11 | 0.0317 |
| GO.0023057 | negative regulation of signaling | 7 | 0.0329 |
| GO.0010648 | negative regulation of cell communication | 7 | 0.0338 |
| GO.0048010 | vascular endothelial growth factor receptor | 3 | 0.0338 |
| | signaling pathway | | |
| GO.0072006 | nephron development | 3 | 0.0338 |
| GO.1902533 | positive regulation of intracellular signal | 6 | 0.0338 |
| | transduction | | |
| GO.0001932 | regulation of protein phosphorylation | 7 | 0.0339 |
| GO.0022414 | reproductive process | 7 | 0.0339 |
| GO.0001959 | regulation of cytokine-mediated signaling | 3 | 0.0341 |
| | pathway | | |
| GO.0060341 | regulation of cellular localization | 7 | 0.0348 |
| GO.0060397 | JAK-STAT cascade involved in growth hormone | 2 | 0.0348 |
| | signaling pathway | | |
| GO.0007160 | cell-matrix adhesion | 3 | 0.0358 |
| GO.0060334 | regulation of interferon-gamma-mediated | 2 | 0.0365 |
| | signaling pathway | | |
| GO.2000352 | negative regulation of endothelial cell apoptotic | 2 | 0.0365 |
| | process | | |
| GO.0001655 | urogenital system development | 4 | 0.0373 |
| GO.0007417 | central nervous system development | 6 | 0.0378 |
| GO.0032870 | cellular response to hormone stimulus | 5 | 0.0378 |
| GO.0072378 | blood coagulation, fibrin clot formation | 2 | 0.0378 |
| GO.0001525 | angiogenesis | 4 | 0.0393 |
| GO.0032386 | regulation of intracellular transport | 5 | 0.0457 |
| GO.0097305 | response to alcohol | 4 | 0.0469 |
| GO.0001953 | negative regulation of cell-matrix adhesion | 2 | 0.0481 |
| GO.0002823 | negative regulation of adaptive immune response | 2 | 0.0481 |
| | based on somatic recombination of immune | | |
| | receptors built from immunoglobulin superfamily | | |
| | domains | | |
| GO.0060612 | adipose tissue development | 2 | 0.0481 |
| GO.0070374 | positive regulation of ERK1 and ERK2 cascade | 3 | 0.0493 |

TABLE 5
List of myeloid cancer genes from Papaemmanuil et al. 2016

| | | VAF | VAF | VAF | VAF | VAF |
|---|---|---|---|---|---|---|
| Symbol | Ensembl ID | Patient | Xenograft 1 | Xenograft 2 | Xenograft 3 | Xenograft 4 |
| ABCA12 | ENSG00000144452 | ND | ND | ND | ND | ND |
| ABL1 | ENSG00000097007 | ND | ND | ND | ND | ND |
| ACTR5 | ENSG00000101442 | ND | ND | ND | ND | ND |
| ARHGAP26 | ENSG00000145819 | ND | ND | ND | ND | ND |
| ASXL1 | ENSG00000171456 | ND | ND | ND | ND | ND |
| ATRX | ENSG00000085224 | ND | ND | ND | ND | ND |
| ATXN7L1 | ENSG00000146776 | ND | ND | ND | ND | ND |
| BCOR | ENSG00000183337 | ND | ND | ND | ND | ND |
| BRAF | ENSG00000157764 | ND | ND | ND | ND | ND |
| CBL | ENSG00000110395 | ND | ND | ND | ND | ND |
| CBLB | ENSG00000114423 | ND | ND | ND | ND | ND |
| CBLC | ENSG00000142273 | ND | ND | ND | ND | ND |
| CD101 | ENSG00000134256 | ND | ND | ND | ND | ND |
| CDH1 | ENSG00000039068 | ND | ND | ND | 0.09 | ND |
| CDKN1B | ENSG00000111276 | ND | ND | ND | ND | ND |
| CDKN2A | ENSG00000147889 | ND | ND | ND | ND | ND |
| CDKN2B | ENSG00000147883 | ND | ND | ND | ND | ND |
| CEBPA | ENSG00000245848 | ND | ND | ND | ND | ND |
| CHGA | ENSG00000100604 | ND | ND | ND | ND | ND |
| CREBBP | ENSG00000005339 | ND | ND | ND | ND | ND |
| CSF1R | ENSG00000182578 | ND | ND | ND | ND | ND |
| CSF2 | ENSG00000164400 | ND | ND | ND | ND | ND |
| CTNNA1 | ENSG00000044115 | ND | ND | ND | ND | ND |
| CUX1 | ENSG00000160967 | ND | ND | ND | ND | ND |
| DDX18 | ENSG00000088205 | ND | ND | ND | ND | ND |
| DNMT1 | ENSG00000130816 | ND | ND | ND | ND | ND |
| DNMT3A | ENSG00000119772 | ND | ND | ND | ND | ND |
| EGFR | ENSG00000146648 | ND | ND | ND | ND | ND |
| ELF1 | ENSG00000120690 | ND | ND | ND | ND | ND |
| EP300 | ENSG00000100393 | ND | ND | ND | ND | ND |
| ERG | ENSG00000157554 | ND | ND | ND | ND | ND |
| ETV6 | ENSG00000139083 | ND | ND | ND | ND | ND |
| MECOM | ENSG00000085276 | ND | ND | ND | ND | ND |
| EZH2 | ENSG00000106462 | ND | ND | ND | ND | ND |
| FAM175B | ENSG00000165660 | ND | ND | ND | ND | ND |
| FBXW7 | ENSG00000109670 | ND | ND | ND | ND | ND |
| FLT3 | ENSG00000122025 | ND | ND | ND | ND | ND |
| GATA1 | ENSG00000102145 | ND | ND | ND | ND | ND |
| GATA2 | ENSG00000179348 | ND | ND | ND | ND | ND |
| GNAS | ENSG00000087460 | ND | ND | ND | ND | ND |
| HIPK2 | ENSG00000064393 | ND | ND | ND | ND | ND |
| HRAS | ENSG00000174775 | ND | ND | ND | ND | ND |
| HMGA2 | ENSG00000149948 | ND | ND | ND | ND | ND |
| IDH1 | ENSG00000138413 | ND | ND | ND | ND | ND |
| IDH2 | ENSG00000182054 | ND | ND | ND | ND | ND |
| IKZF1 | ENSG00000185811 | ND | ND | ND | ND | ND |
| INVS | ENSG00000119509 | ND | ND | ND | ND | ND |
| IRF1 | ENSG00000125347 | ND | ND | ND | ND | ND |
| JAK2 | ENSG00000096968 | ND | ND | ND | ND | ND |
| JAK3 | ENSG00000105639 | ND | ND | ND | ND | ND |
| KDM2B | ENSG00000089094 | ND | ND | ND | ND | ND |
| KDM5A | ENSG00000073614 | ND | ND | ND | ND | ND |
| KDM6A | ENSG00000147050 | ND | ND | ND | ND | ND |
| KIT | ENSG00000157404 | ND | ND | ND | ND | ND |
| KRAS | ENSG00000133703 | ND | ND | ND | ND | ND |
| LCORL | ENSG00000178177 | ND | ND | ND | ND | ND |
| LILRA3 | ENSG00000170866 | ND | ND | ND | ND | ND |
| MAP2K5 | ENSG00000137764 | ND | ND | ND | ND | ND |
| MET | ENSG00000105976 | ND | ND | ND | ND | ND |
| MLL | ENSG00000118058 | ND | ND | ND | ND | ND |
| MLL2 | ENSG00000167548 | ND | ND | ND | ND | ND |
| MLL3 | ENSG00000055609 | 0.11 | 0.10 | 0.11 | 0.13 | 0.05 |
| MLL5 | ENSG00000005483 | ND | ND | ND | ND | ND |
| MMD2 | ENSG00000136297 | ND | ND | ND | ND | ND |
| MN1 | ENSG00000169184 | ND | ND | ND | ND | ND |
| MPL | ENSG00000117400 | ND | ND | ND | ND | ND |
| MTAP | ENSG00000099810 | ND | ND | ND | ND | ND |
| MYC | ENSG00000136997 | ND | ND | ND | ND | ND |
| NF1 | ENSG00000196712 | ND | ND | ND | ND | ND |
| NLRP1 | ENSG00000091592 | ND | ND | ND | ND | ND |
| NOTCH1 | ENSG00000148400 | ND | ND | ND | ND | ND |
| NPM1 | ENSG00000181163 | ND | ND | ND | ND | ND |
| NR5A1 | ENSG00000136931 | ND | ND | ND | ND | ND |
| NRAS | ENSG00000213281 | 1.00 | 1.00 | 1.00 | 1.00 | 1.00 |
| NRD1 | ENSG00000078618 | ND | ND | ND | ND | ND |
| NSD1 | ENSG00000165671 | ND | ND | ND | ND | ND |
| NUP98 | ENSG00000110713 | ND | ND | ND | ND | ND |
| OCA2 | ENSG00000104044 | ND | ND | ND | ND | ND |
| PDGFRA | ENSG00000134853 | ND | ND | ND | ND | ND |
| PHF12 | ENSG00000109118 | ND | ND | ND | ND | ND |
| PHF6 | ENSG00000156531 | 1.00 | 1.00 | 0.98 | 1.00 | 1.00 |
| PKP3 | ENSG00000184363 | ND | ND | ND | ND | ND |
| PRDX2 | ENSG00000167815 | ND | ND | ND | ND | ND |
| PRPF40B | ENSG00000110844 | ND | ND | ND | ND | ND |
| PTEN | ENSG00000171862 | ND | ND | ND | ND | ND |
| PTPN11 | ENSG00000179295 | ND | ND | ND | ND | ND |
| RAD21 | ENSG00000164754 | ND | ND | ND | ND | ND |
| RAD50 | ENSG00000113522 | ND | ND | ND | ND | ND |
| RB1 | ENSG00000139687 | ND | ND | ND | ND | ND |
| RINT1 | ENSG00000135249 | ND | ND | ND | ND | ND |
| RORC | ENSG00000143365 | ND | ND | ND | ND | ND |
| RUNX1 | ENSG00000159216 | ND | ND | ND | ND | ND |
| RUNX1T1 | ENSG00000079102 | ND | ND | ND | ND | ND |
| SF1 | ENSG00000168066 | ND | ND | ND | ND | ND |
| SF3A1 | ENSG00000099995 | ND | ND | ND | ND | ND |
| SF3B1 | ENSG00000115524 | ND | ND | ND | ND | ND |
| SH2B3 | ENSG00000111252 | ND | ND | ND | ND | ND |
| SOCS1 | ENSG00000185338 | ND | ND | ND | ND | ND |
| SPI1 | ENSG00000066336 | ND | ND | ND | ND | ND |
| SRPK2 | ENSG00000135250 | ND | ND | ND | ND | ND |
| SRSF2 | ENSG00000161547 | ND | ND | ND | ND | ND |
| STAG2 | ENSG00000101972 | ND | ND | ND | ND | ND |
| STK17B | ENSG00000081320 | ND | ND | ND | ND | ND |
| TCF4 | ENSG00000196628 | ND | ND | ND | ND | ND |
| TET1 | ENSG00000138336 | ND | ND | ND | ND | ND |
| TET2 | ENSG00000168769 | ND | ND | ND | ND | ND |
| TP53 | ENSG00000141510 | ND | ND | ND | ND | ND |
| U2AF1 | ENSG00000160201 | ND | ND | ND | ND | ND |
| U2AF2 | ENSG00000063244 | ND | ND | ND | ND | ND |
| WT1 | ENSG00000184937 | ND | ND | ND | ND | ND |
| ZEB2 | ENSG00000169554 | ND | ND | ND | ND | ND |
| ZRSR2 | ENSG00000169249 | ND | ND | ND | ND | ND |

TABLE 6
Leukemic chimerism levels following DRD2 antagonist therapy

| Patient | | −DRD2 | +DRD2 | Sample | |
|---|---|---|---|---|---|
| sample | AML cell state | Antagonist | Antagonist | size | p value |
| AML #5 | Therapy-naive | 14.9 ± 3.8 | 12.4 ± 2.2 | 4, 6 | 0.50 |
| AML #5 | LRCs | 2.7 ± 0.6 | 1.4 ± 0.3 | 12, 9 | 0.19 |
| AML #10 | LRCs | 11.2 ± 2.2 | 16.9 ± 8.7 | 4, 4 | 1.00 |
| AML #11 | LRCs | 0.9 ± 0.7 | 1.4 ± 1.4 | 7, 7 | 1.00 |

TABLE 7
Description of xenograft assays

| | | Mouse# | Group |
|---|---|---|---|
| FIG. ID | Xenograft source | per group | description |
| 1D | Patient 2 | 5 | treatment group |
| 1F, right panel | Patient 3 | 4-5 | treatment group |
| | Patient 2 | 4-5 | cell subfraction |
| 1J | Patient 2 | 3-4 | treatment group |
| 2A | Patient 3 | 3-4 | treatment group |
| | Patient 2 and 3 | 3-8 | response group |
| 2C | Patient 2, 3 and 5 | 6-12 | time point |
| 2D | healthy donor MPB | 4 | time point |
| 2E | Healthy donor CB | 5 | time point |
| | Patient 3 | 6-7 | treatment group |
| 2F | healthy donor MPB | 4 | treatment group |
| 3A, top panel | Patient 3 | 6 | time point |
| 3B, top panel | Patient 2 | 6 | time point |
| 3C | Patient 6 | mouse# shown | treatment group |
| | | in Table 3 | |
| 6A-F | Patient 5 | mouse# shown | treatment group |
| | | in schematic | |

### REFERENCES

Bologna-Molina, R., Mosqueda-Taylor, A., Molina-Frechero, N., Mori-Estevez, A. D., and Sanchez-Acuna, G. (2013). Comparison of the value of PCNA and Ki-67 as markers of cell proliferation in ameloblastic tumors. Med Oral Patol Oral Cir Bucal 18, e174-179.
Boyd, A. L., Campbell, C. J. V., Hopkins, C. I., Fiebig-Comyn, A., Russell, J., Ulemek, J., Foley, R., Leber, B., Xenocostas, A., Collins, T. J., et al. (2014). Niche displacement of human leukemic stem cells uniquely allows their competitive replacement with healthy HSPCs. JEM 211, 1925-1935.
Burke, P. J., Karp, J. E., Braine, H. G., and Vaughan, W. P. (1977). Timed sequential therapy of human leukemia based upon the response of leukemic cells to humoral growth factors. Cancer Res 37, 2138-2146.
Cannistra, S. A., Groshek, P., and Griffin, J. D. (1989). Granulocyte-macrophage colony-stimulating factor enhances the cytotoxic effects of cytosine arabinoside in acute myeloblastic leukemia and in the myeloid blast crisis phase of chronic myeloid leukemia. Leukemia 3, 328-334.
Cao, Y. A., Wagers, A. J., Karsunky, H., Zhao, H., Reeves, R., Wong, R. J., Stevenson, D. K., Weissman, I. L., and Contag, C. H. (2008). Heme oxygenase-1 deficiency leads to disrupted response to acute stress in stem cells and progenitors. Blood 112, 4494-4502.
Ding, L., Ley, T. J., Larson, D. E., Miller, C. A., Koboldt, D. C., Welch, J. S., Ritchey, J. K., Young, M. A., Lamprecht, T., McLellan, M. D., et al. (2012). Clonal evolution in relapsed acute myeloid leukaemia revealed by whole-genome sequencing. Nature 481, 506-510.
Ebinger, S., Ozdemir, E. Z., Ziegenhain, C., Tiedt, S., Castro Alves, C., Grunert, M., Dworzak, M., Lutz, C., Turati, V. A., Enver, T., et al. (2016). Characterization of Rare, Dormant, and Therapy-Resistant Cells in Acute Lymphoblastic Leukemia. Cancer Cell 30, 849-862.
Eppert, K., Takenaka, K., Lechman, E. R., Waldron, L., Nilsson, B., van Galen, P., Metzeler, K. H., Poeppl, A., Ling, V., Beyene, J., et al. (2011). Stem cell gene expression programs influence clinical outcome in human leukemia. Nat Med 17, 1086-1093.
Estey, E., and Dohner, H. (2006). Acute myeloid leukaemia. Lancet 368, 1894-1907.
Etxabe, A., Lara-Castillo, M. C., Cornet-Masana, J. M., Banus-Mulet, A., Nomdedeu, M., Torrente, M. A., Pratcorona, M., Diaz-Beya, M., Esteve, J., and Risueno, R. M. (2017). Inhibition of serotonin receptor type 1 in acute myeloid leukemia impairs leukemia stem cell functionality: a promising novel therapeutic target. Leukemia 1, 2288-2302.
Farge, T., Saland, E., de Toni, F., Aroua, N., Hosseini, M., Perry, R., Bosc, C., Sugita, M., Stuani, L., Fraisse, M., et al. (2017). Chemotherapy Resistant Human Acute Myeloid Leukemia Cells are Not Enriched for Leukemic Stem Cells but Require Oxidative Metabolism. Cancer Discovery 7, 716-735.
Finn, R. S., Martin, M., Rugo, H. S., Jones, S., Im, S. A., Gelmon, K., Harbeck, N., Lipatov, O. N., Walshe, J. M., Moulder, S., et al. (2016). Palbociclib and Letrozole in Advanced Breast Cancer. N Engl J Med 375, 1925-1936.
Griessinger, E., Anjos-Afonso, F., Pizzitola, I., Rouault-Pierre, K., Vargaftig, J., Taussig, D., Gribben, J., Lassailly, F., and Bonnet, D. (2014). A niche-like culture system allowing the maintenance of primary human acute myeloid leukemia-initiating cells: a new tool to decipher their chemoresistance and self-renewal mechanisms. Stem Cells Transl Med 3, 520-529.
Hackl, H., Steinleitner, K., Lind, K., Hofer, S., Tosic, N., Pavlovic, S., Suvajdzic, N., Sill, H., and Wieser, R. (2015). A gene expression profile associated with relapse of cytogenetically normal acute myeloid leukemia is enriched for leukemia stem cell genes. Leuk Lymphoma 56, 1126-1128.
Hirsch, P., Zhang, Y., Tang, R., Joulin, V., Boutroux, H., Pronier, E., Moatti, H., Flandrin, P., Marzac, C., Bories, D., et al. (2016). Genetic hierarchy and temporal variegation in the clonal history of acute myeloid leukaemia. Nat Commun 7, 12475.
Ho, T. C., LaMere, M., Stevens, B. M., Ashton, J. M., Myers, J. R., O'Dwyer, K. M., Liesveld, J. L., Mendler, J. H., Guzman, M., Morrissette, J. D., et al. (2016). Evolution of acute myelogenous leukemia stem cell properties after treatment and progression. Blood 128, 1671-1678.
Huang, S. (2014). The war on cancer: lessons from the war on terror. Front Oncol 4, 293.
Ishikawa, F., Yoshida, S., Saito, Y., Hijikata, A., Kitamura, H., Tanaka, S.,
Nakamura, R., Tanaka, T., Tomiyama, H., Saito, N., et al. (2007).
Chemotherapy-resistant human AML stem cells home to and engraft within the bone-marrow endosteal region. Nat Biotechnol 25, 1315-1321.
Jordan, C. T., Guzman, M. L., and Noble, M. (2006). Cancer stem cells. N Engl J Med 355, 1253-1261.
Kronke, J., Bullinger, L., Teleanu, V., Tschurtz, F., Gaidzik, V. I., Kuhn, M. W., Rucker, F. G., Holzmann, K., Paschka, P., Kapp-Schworer, S., et al. (2013). Clonal evolution in relapsed NPM1-mutated acute myeloid leukemia. Blood 122, 100-108.
Kurtova, A. V., Xiao, J., Mo, Q., Pazhanisamy, S., Krasnow, R., Lerner, S. P., Chen, F., Roh, T. T., Lay, E., Ho, P. L., et al. (2015). Blocking PGE2-induced tumour repopulation abrogates bladder cancer chemoresistance. Nature 517, 209-213.
Levine, J. H., Simonds, E. F., Bendall, S. C., Davis, K. L., Amir el, A. D., Tadmor, M. D., Litvin, O., Fienberg, H. G., Jager, A., Zunder, E. R., et al. (2015). Data-Driven Phenotypic Dissection of AML Reveals Progenitor-like Cells that Correlate with Prognosis. Cell 162, 184-197.
Liliemark, J. O., Gahrton, G., Paul, C. Y., and Peterson, C. O. (1987). ara-C in plasma and ara-CTP in leukemic cells after subcutaneous injection and continuous intravenous infusion of ara-C in patients with acute nonlymphoblastic leukemia. Semin Oncol 14, 167-171.
Ng, S. W. K., Mitchell, A., Kennedy, J. A., Chen, W. C., McLeod, J., Ibrahimova, N., Arruda, A., Popescu, A., Gupta, V., Schimmer, A. D., et al. (2016). A 17-gene stemness score for rapid determination of risk in acute leukaemia. Nature 540, 433-437.
Papaemmanuil, E., Gerstung, M., Bullinger, L., Gaidzik, V. I., Paschka, P., Roberts, N. D., Potter, N. E., Heuser, M., Thol, F., Bolli, N., et al. (2016). Genomic Classification and Prognosis in Acute Myeloid Leukemia. N Engl J Med 374, 2209-2221.
Passaro, D., Di Tullio, A., Abarrategi, A., Rouault-Pierre, K., Foster, K., Ariza-McNaughton, L., Montaner, B., Chakravarty, P., Bhaw, L., Diana, G., et al. (2017). Increased Vascular Permeability in the Bone Marrow Microenvironment Contributes to Disease Progression and Drug Response in Acute Myeloid Leukemia. Cancer Cell 32, 324-341.
Pollyea, D. A., Gutman, J. A., Gore, L., Smith, C. A., and Jordan, C. T. (2014). Targeting acute myeloid leukemia stem cells: a review and principles for the development of clinical trials. Haematologica 99, 1277-1284.
Reese, N. D., and Schiller, G. J. (2013). High-dose cytarabine (HD araC) in the treatment of leukemias: a review. Curr Hematol Malig Rep 8, 141-148.
Roth, A., Khattra, J., Yap, D., Wan, A., Laks, E., Biele, J., Ha, G., Aparicio, S., Bouchard-Cote, A., and Shah, S. P. (2014). PyClone: statistical inference of clonal population structure in cancer. Nat Methods 11, 396-398.
Sachlos, E., Risueno, R. M., Laronde, S., Shapovalova, Z., Lee, J. H., Russell, J., Malig, M., McNicol, J. D., Fiebig-Comyn, A., Graham, M., et al. (2012). Identification of drugs including a dopamine receptor antagonist that selectively target cancer stem cells. Cell 149, 1284-1297.
Saito, Y., Uchida, N., Tanaka, S., Suzuki, N., Tomizawa-Murasawa, M., Sone, A., Najima, Y., Takagi, S., Aoki, Y., Wake, A., et al. (2010). Induction of cell cycle entry eliminates human leukemia stem cells in a mouse model of AML. Nat Biotechnol 28, 275-280.
Seeman, P., and Lee, T. (1975). Antipsychotic drugs: direct correlation between clinical potency and presynaptic action on dopamine neurons. Science 188, 1217-1219.
Shlush, L. I., Mitchell, A., Heisler, L., Abelson, S., Ng, S. W. K., Trotman-Grant, A., Medeiros, J. J. F., Rao-Bhatia, A., Jaciw-Zurakowsky, I., Marke, R., et al. (2017). Tracing the origins of relapse in acute myeloid leukaemia to stem cells. Nature 547, 104-108.
Sun, J., Ramos, A., Chapman, B., Johnnidis, J. B., Le, L., Ho, Y. J., Klein, A., Hofmann, O., and Camargo, F. D. (2014). Clonal dynamics of native haematopoiesis. Nature 514, 322-327.
Thomas, D., and Majeti, R. (2017). Optimizing Next-Generation AML Therapy: Activity of Mutant IDH2 Inhibitor AG-221 in Preclinical Models. Cancer Discov 7, 459-461.
Thomas, D., and Majeti, R. (2017). Biology and relevance of human acute myeloid leukemia stem cells. Blood 129, 1577-1585.
Ueda, T., Yoshida, M., Yoshino, H., Kobayashi, K., Kawahata, M., Ebihara, Y., Ito, M., Asano, S., Nakahata, T., and Tsuji, K. (2001). Hematopoietic capability of CD34+ cord blood cells: a comparison with CD34+ adult bone marrow cells. Int J Hematol 73, 457-462.
Wilson, A., Laurenti, E., Oser, G., van der Wath, R. C., Blanco-Bose, W., Jaworski, M., Offner, S., Dunant, C. F., Eshkind, L., Bockamp, E., et al. (2008). Hematopoietic stem cells reversibly switch from dormancy to self-renewal during homeostasis and repair. Cell 135, 1118-1129.
Zhou, B. O., Ding, L., and Morrison, S. J. (2015). Hematopoietic stem and progenitor cells regulate the regeneration of their niche by secreting Angiopoietin-1. Elife 4, e05521.
Zuber, J., Radtke, I., Pardee, T. S., Zhao, Z., Rappaport, A. R., Luo, W., McCurrach, M. E., Yang, M. M., Dolan, M. E., Kogan, S. C., et al. (2009). Mouse models of human AML accurately predict chemotherapy response. Genes Dev 23, 877-889.

1. A method of determining a prognosis for a subject who has completed a cytotoxic treatment for leukemia, the method comprising:
determining a level of one or more biomarkers listed in Table 4a in a test sample obtained from the subject after completing the cytotoxic treatment for leukemia; and
comparing the level of the one or more biomarkers in the test sample to one or more control levels,
wherein a difference or similarity in the level of the one or more biomarkers in the test sample compared to the one or more control levels is indicative of whether the subject has an increased or decreased risk of relapsing leukemia.

2. The method of claim 1, wherein the one or more biomarkers comprise biomarkers selected from SLC2A2, DRD2, FASLG and FUT3.

3. The method of claim 1, comprising determining a level of SLC2A2 in the test sample wherein an increased level of SLC2A2 of in the test sample compared to the control level is indicative of an increased risk of relapsing leukemia.

4. The method of claim 1, wherein the test sample comprises leukemic cells, optionally CD45+ cells.

5. The method of claim 1, comprising generating a biomarker expression profile for the test sample based on the levels of a plurality of the biomarkers in the test sample, and comparing the biomarker expression profile for the test sample to a control biomarker expression profile, wherein the control biomarker expression profile is representative of Leukemic Regenerating Cells (LRCs) and a similarity in the biomarker expression profile of the test sample and the control biomarker expression profile is indicative of an increased risk of relapsing leukemia.

6. The method of claim 1, wherein the test sample is obtained from the subject between about 10 days and 40 days after completing the cytotoxic treatment.

7. The method of claim 1, the method comprising:
generating a biomarker expression profile for the test sample from the subject based on the level of one or more biomarkers listed in Table 4a; and
classifying, on a computer, whether the subject has a good prognosis and a low risk of relapsing leukemia or a poor prognosis and a high risk of relapsing leukemia, based on the biomarker expression profile for the test sample.

8. A method of treating a subject having leukemia, comprising determining a prognosis of the subject according to the method of claim 1, and providing a suitable treatment to the subject in need thereof according to the prognosis determined.

9. A method of detecting Leukemic Regenerating Cells (LRCs) in a test sample, the method comprising:
detecting a level of one or more biomarkers listed in Table 4A in the test sample; and
comparing the level of the one or more biomarkers in the test sample to one or more control levels.

10. The method of claim 9, wherein the one or more control levels are representative of the level of the one or more biomarkers in LRCs and similarity between the level of the one or more biomarkers in the test sample and the one or more control levels is indicative of the presence of LRCs in the test sample.

11. The method of claim 9, further comprising isolating the LRCs from the test sample.

12. An isolated population of LRCs produced according to the method of claim 11, wherein the cells express one or more of the biomarkers listed in Table 4a.

13. A cell culture comprising the population of LRCs of claim 12 and a culture media, wherein the culture media comprises serum from a subject previously exposed to a cytotoxic therapy, optionally cytarabine.

14. A method of screening a test agent for use in preventing or inhibiting relapsing leukemia, the method comprising:
contacting the test agent with the LRCs of claim 12; and
detecting a biological effect of the test agent on the LRCs.

15. The method of claim 14, wherein the biological effect comprises a reduction in the level of LRCs and the test agent is identified as a candidate for preventing or inhibiting relapsing leukemia.

16. A method of treating leukemia in a subject in need thereof, the method comprising administering to the subject an agent that targets Leukemic Regenerating Cells (LRCs), wherein the subject has completed a cytotoxic treatment for leukemia.

17. The method of claim 16, wherein the agent selectively targets LRCs relative to HRCs.

18. The method of claim 16, comprising administering the agent that targets LRCs to the subject between 10 days and 40 days after completing the cytotoxic treatment for leukemia.

19. The method of claim 16, wherein the agent that targets LRCs is an antagonist for a gene or protein selected from VIPR2, PAFAH1B3, LPAR3, FGFR2, CLPS, KCNA4, BAAT, HTR4, NALCN, CARTPT, HTR1B, DRD2, BDKRB1, KCNJ10, SLC36A2, GRM5, KCNA10, SLC2A2 and PLG.

20. The method of claim 16, wherein the agent that targets LRCs is a DRD2 antagonist, optionally thioridazine.
